SEQUENCE LISTING (45 sequences)

SEQ ID NO: 1 - protein, 563 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLP

SEQ ID NO: 2 - protein, 320 aa, synthetic construct
SETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - DNA, 1689 bp, synthetic construct
  source 1..1689: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcct

SEQ ID NO: 4 - DNA, 960 bp, synthetic construct
  source 1..960: mol_type = other DNA
agtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg

SEQ ID NO: 5 - protein, 150 aa, synthetic construct
HTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 6 - protein, 151 aa, synthetic construct
MHTLYAPGGYDIMGYLRQIRNRPNPQVELGPVDTSCALILCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 7 - DNA, 450 bp, synthetic construct
  source 1..450: mol_type = other DNA
cacactctttacgcccctggaggatacgacattatgggatatttggatcagattgggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatccggaaggccatcgaccggaacgccgaggtccaggtggaggtggtcaactttaagaagaacggccagcggttcgtgaactttctgaccatcattccggtccgggatgaaaccggagagtacagatactccatgggattccagtgcgaaaccgaa

SEQ ID NO: 8 - DNA, 453 bp, synthetic construct
  source 1..453: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaa

SEQ ID NO: 9 - DNA, 2157 bp, synthetic construct
  source 1..2157: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctggcggttctggaggtcacactctttacgcccctggaggatacgacattatgggatatttggatcagattgggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatccggaaggccatcgaccggaacgccgaggtccaggtggaggtggtcaactttaagaagaacggccagcggttcgtgaactttctgaccatcattccggtccgggatgaaaccggagagtacagatactccatgggattccagtgcgaaaccgaataa

SEQ ID NO: 10 - DNA, 2157 bp, synthetic construct
  source 1..2157: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctggcggttctggaggtcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaataa

SEQ ID NO: 11 - DNA, 1431 bp, synthetic construct
  source 1..1431: mol_type = other DNA
atgcacactctttacgcccctggaggatacgacattatgggatatttggatcagattgggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatccggaaggccatcgaccggaacgccgaggtccaggtggaggtggtcaactttaagaagaacggccagcggttcgtgaactttctgaccatcattccggtccgggatgaaaccggagagtacagatactccatgggattccagtgcgaaaccgaaggcggttctggaggtagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 12 - DNA, 1431 bp, synthetic construct
  source 1..1431: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaaggcggttctggaggtagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 13 - protein, 297 aa, Bacillus megaterium
MGNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSS

SEQ ID NO: 14 - protein, 273 aa, Streptomyces antibioticus
MTVRKNQASLTAEEKRRFVAALLELKRTGRYDAFVTTHNAFILGDTDNGERTGHRSPSFLPWHRRFLLEFERALQSVDASVALPYWDWSADRSTRSSLWAPDFLGGTGRSRDGQVMDGPFAASAGNWPINVRVDGRTFLRRALGAGVSELPTRAEVDSVLAMATYDMAPWNSGSDGFRNHLEGWRGVNLHNRVHVWVGGQMATGVSPNDPVFWLHHAYIDKLWAEWQRRHPSSPYLPGGGTPNVVDLNETMKPWNDTTPAALLDHTRHYTFDV

SEQ ID NO: 15 - protein, 609 aa, Rhizobium etli
MAWLVGKPSLERSWNAILSFPESGFQLECRNTIGSSVFSSHFTLHFRVARRLLHFSCRRFTETQKEPTQALWWCELPTAPAPRRRGTGLKAALILAKDNSNPRESKMSITRRHVIVQGGVIAAGLLASGLPGTKAFAQIPSIPWRRSLQGLAWNDPIIETYRDAVRLLNALPASDKFNWVNLSKIHGSGDVVKYCPHGNWYFLPWHRAYTAMYERIVRHVTKNNDFAMPFWDWTDNPYLPEVFTMQKTPDGKDNPLYVSSRTWPITQPMPDNIVGPQVLNTILTAKPYEVFGTTRPEGQNSLDPSWVTTSSGTQGALEYTPHNQVHNNIGGWMPEMSSPRDPIFFMHHCNIDRIWATWNLRNANSTDRLWADMPFTDNFYDVDGNFWSPKVSDLYVPEELGYNYGFRTYFKVAAASAKTLALNDKLTSVIAATATDAAIAGVTTTSTDNSKAATENVPLSLPIKIPAGALQEIVRQPPLPSGMDTMDFGAAQEQAASAPRVLAFLRDVEITSASTTSVRVFLGKNDLKADTPVTGPHYVGSFAVLGHDGDHHRKPSFVLDLTDAIQRVYGGRGQTDGEAIDLQLIPVGSGAGKPGAVEPAKLEIAIVSA

SEQ ID NO: 16 - DNA, 35 bp, synthetic construct
  source 1..35: mol_type = other DNA
ttgacagctagctcagtcctaggtattgtgctagc

SEQ ID NO: 17 - DNA, 36 bp, synthetic construct
  source 1..36: mol_type = other DNA
ggatcttagctactagagaaagaggagaaatactag

SEQ ID NO: 18 - DNA, 102 bp, synthetic construct
  source 1..102: mol_type = other DNA
tagcatttttatccataagattagcggatcctacctgacgctttttatcgcaactctctactgtttctccatacccgtttttttggaaggaggtaaagatct

SEQ ID NO: 19 - DNA, 55 bp, synthetic construct
  source 1..55: mol_type = other DNA
acctgtaggatcgtacaggtttacgcaagaaaatggtttgttatagtcgaataaa

SEQ ID NO: 20 - DNA, 54 bp, synthetic construct
  source 1..54: mol_type = other DNA
tccctatcagtgatagagattgacatccctatcagtgatagagatactgagcac

SEQ ID NO: 21 - DNA, 37 bp, synthetic construct
  source 1..37: mol_type = other DNA
tggtgcaaaacctttcgcggtatggcatgatagcgcc

SEQ ID NO: 22 - DNA, 879 bp, synthetic construct
  source 1..879: mol_type = other DNA
atggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 23 - DNA, 753 bp, synthetic construct
  source 1..753: mol_type = other DNA
atgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaa

SEQ ID NO: 24 - DNA, 624 bp, synthetic construct
  source 1..624: mol_type = other DNA
atgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaa

SEQ ID NO: 25 - DNA, 1083 bp, synthetic construct
  source 1..1083: mol_type = other DNA
atgaaaccagtaacgttatacgatgtcgcagagtatgccggtgtctcttatatgaccgtttcccgcgtggtgaaccaggccagccacgtttctgcgaaaacgcgggaaaaagtggaagcggcgatggtggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattatttactcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 26 - DNA, 207 bp, synthetic construct
  source 1..207: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgcc

SEQ ID NO: 27 - protein, 69 aa, synthetic construct
MNTINIAKNDFSDIELAAIPFNTLADHYGERLAREQLALEHESYEMGEARFRKMFERQLKAGEVADNAA

SEQ ID NO: 28 - DNA, 672 bp, synthetic construct
  source 1..672: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccggcggttctggaggtcacactctttacgcccctggaggatacgacattatgggatatttggatcagattgggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatccggaaggccatcgaccggaacgccgaggtccaggtggaggtggtcaactttaagaagaacggccagcggttcgtgaactttctgaccatcattccggtccgggatgaaaccggagagtacagatactccatgggattccagtgcgaaaccgaa

SEQ ID NO: 29 - protein, 224 aa, synthetic construct
MNTINIAKNDFSDIELAAIPFNTLADHYGERLAREQLALEHESYEMGEARFRKMFERQLKAGEVADNAAGGSGGHTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 30 - DNA, 453 bp, synthetic construct
  source 1..453: mol_type = other DNA
atgcacactctttacgcccctggaggatacgacattatgggatatttgcggcagattaggaaccgcccaaaccctcaggtcgaactggggcctgtggacacgtcatgtgccctggtcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaa

SEQ ID NO: 31 - protein, 151 aa, synthetic construct
MHTLYAPGGYDIMGYLRQIRNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 32 - DNA, 2445 bp, synthetic construct
  source 1..2445: mol_type = other DNA
gccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 33 - protein, 814 aa, synthetic construct
AKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 34 - DNA, 960 bp, synthetic construct
  source 1..960: mol_type = other DNA
agtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg

SEQ ID NO: 35 - protein, 320 aa, synthetic construct
SETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 36 - DNA, 1428 bp, synthetic construct
  source 1..1428: mol_type = other DNA
atgcacactctttacgcccctggaggatacgacattatgggatatttgcggcagattaggaaccgcccaaaccctcaggtcgaactggggcctgtggacacgtcatgtgccctggtcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaaggcggttctggaggtagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg

SEQ ID NO: 37 - protein, 476 aa, synthetic construct
MHTLYAPGGYDIMGYLRQIRNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGGSGGSETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 38 - DNA, 2913 bp, synthetic construct
  source 1..2913: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaaggcggttctggaggtgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 39 - protein, 970 aa, synthetic construct
MHTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGGSGGAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 40 - DNA, 1428 bp, synthetic construct
  source 1..1428: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaaggcggttctggaggtagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg

SEQ ID NO: 41 - protein, 476 aa, synthetic construct
MHTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGGSGGSETVQDIYGIVAKKVNEILQADAINGTDNEVVTVTDENTGEISEKVKLGTKALAGQWLAYGVTRSVTKSSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 42 - DNA, 1428 bp, synthetic construct
  source 1..1428: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccggtggtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaaggcggttctggaggtagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg

SEQ ID NO: 43 - protein, 476 aa, synthetic construct
MHTLYAPGGYDIMGYLRQIRNRPNPQVELGPVDTSCALILCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGGSGGSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 44 - DNA, 453 bp, synthetic construct
  source 1..453: mol_type = other DNA
atgcacactctttacgcccctggaggatacgacattatgggatatttgcggcagattaggaaccgcccaaaccctcaggtcgaactggggcctgtggacacgtcatgtgccctgatcctgtgcgatctgaagcaaaaggacactccgatcgtctacgcctcggaagccttcttgtatatgaccggatacagcaatgcagaggtgctcggcaggaactgcagattcctgcagtcccccgacgggatggtgaaaccaaagtcgactcgcaaatatgtggactcgaacacgatcaacaccatgcggaaggccatcgaccggaacgccgaggtccaggtggaggtggtcaactttaagaagaacggccagcggttcgtgaactttctgaccatgattccggtccgggatgaaaccggagagtacagatactccatgggattccagtgcgaaaccgaa

SEQ ID NO: 45 - protein, 151 aa, synthetic construct
MHTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

(19) United States
(12) Patent Application Publication
(10) Pub. No.: US 2024/0318215 A1
(43) Pub. Date: Sep. 26, 2024

(54) METHODS AND COMPOSITIONS

(71) Applicant: Imperial College Innovations Ltd, London (GB)

(72) Inventors: Kenneth Thomas Walker, London (GB); Thomas Marc Ellis, London (GB)

(21) Appl. No.: 18/579,094

(22) PCT Filed: Jul. 12, 2022
(86) PCT No.: PCT/GB2022/051794

§ 371 Date: Jan. 12, 2024

(30) Foreign Application Priority Data
Jul. 13, 2021 (GB) 2110091.2

(51) Int. Cl. C12P 19/04; C12N 1/20; C12N 9/02; C12P 17/10

(52) U.S. Cl. CPC C12P 19/04; C12N 1/20; C12N 9/0071; C12P 17/10; C12Y 114/18001

(57) ABSTRACT

The present invention relates to biomaterials, in particular bacterial cellulose and provides means to prepare pigmented cellulose at acidic pH 5.8, wherein cellulose pellicles comprising tyrosinase (EC 1.14.18.1) are melanated using a development solution at pH 6 to 8.5 and comprises tyrosine, cysteine and/or cystine. Further, the invention relates to means of preparing spatially restricted pigmented cellulose using an optogenetic expression system wherein two polymerase or transcription factor domains are split and each linked to a light-inducible dimerization domain. The invention provides corresponding methods and components.

### FIELD OF THE INVENTION

The present invention is in the field of biomaterials.

### BACKGROUND

Biomaterials are appealing targets for exploration; however, the harvesting and farming of many biomaterials are not feasible. Biological systems did not evolve to be easily exploited in industrial processes, they evolved to survive, often producing just enough material necessary to do so. Generations of selective breading have been successful in Increasing biomaterial yields, yet the possibilities of this approach are limited. However, by understanding, engineering and adapting the tools these biological systems use to produce biomaterials, we may be able to construct biomaterials for our own specific aims, for example to be more sustainable. Synthetic biology, a field that applies the approaches of engineering to biological systems, aims to give us such capabilities. To understand how synthetic biology could transform the material space, it must be applied to a system of biomaterial production. Bacterial cellulose, a pure cellulose material produced by many bacteria, but overproduced by the genetically tractable Komagataeibacter rhaeticus may be the best place to explore this potential.

Cellulose is the most abundant polymer on the planet and makes up the majority of common materials like wood, cotton and paper. Cellulose is formed from a linear chain of beta-glucose monomers and is used by biological systems to create a variety of materials. Biological systems do this through controlling the nanoscopic and macroscopic arrangement of cellulose and using cellulose to make composites with other materials.

While plants are by far the most effective producers of cellulose, bacteria have also evolved the ability to synthesise cellulose. In many gram-negative bacteria, cellulose provides a structural role in biofilm formation, where bacteria cooperate to build three-dimensional multicellular structures. Bacteria, such as E. coli and some Pseudomonas species, simultaneously produce cellulose with structural and adhesive proteins, such as curli fibres, to create composite biomaterials.

Bacterially produced cellulose is known by many names: bacterial cellulose, microbial cellulose, nanofibrilliated cellulose, bacterial nanocellulose. Here we will refer to the material as bacterial cellulose (BC).

Among bacteria that produce BC, a small group have evolved the ability to significantly overproduce cellulose. These cellulose overproducers are found within a group of bacteria known as acetic acid bacteria—obligate aerobes that consume sugars and alcohols and produce acetic acid as a by-product.

The cellulose overproducers are found within the genus Komagataeibacter. The most common application of Komagataeibacter is in the production of kombucha, a beverage based on the fermentation of tea by a consortia of Komagataeibacter and various yeast species.

During the fermentation of kombucha a large mass of cellulose and bacteria accumulate at the air/liquid interface. This mass is referred to as a pellicle and is composed of BC. Pellicle growth follows the meniscus of the liquid and will extend as wide as the culturing container. A pellicle can grow to several centimetres in thickness, however since hydrated bacterial cellulose is 99% water, even the thickest pellicles are reduced significantly to less than a millimetre thick when dried (FIG. 18).

The Komagataeibacter pellicle is biofilm like; it acts as an extracellular polysaccharide mesh that contains the bacteria that produce it. However, unlike other biofilms, the Komagataeibacter pellicle is almost purely cellulose.

BC may not seem like an obvious target for biomaterial research, given the abundance of plant cellulose, however BC has many remarkable properties that cast it apart from plant cellulose.

Bacterial cellulose has a hierarchical structure. Single glycan chains crosslink through hydrogen bonding to form fibrils, which then further associate to make fibres. These fibres are typically 40-60 nm in diameter. The thinness of BC fibres classifies the biomaterial as nanocellulose. BC has a high degree of crystallinity, between 50-80%, with the majority of the glycan chains in either a cellulose I or II crystal state. The high degree of crystallinity and ordered structure contributes to the remarkable material properties of BC. BC is strong, with a single nanofiber having a high tensile strength of ˜1 GPa—similar to that of Kevlar. BC fibres are stiff under strain, with a Young's modulus of 114 GPa, similar to that of bronze. The high internal surface area and availability of hydroxyl groups within BC, means BC is highly hydrophilic, with a water retention of up 1000% its own weight. Additionally, only 10% of that water acts a free bulk water, meaning BC stays hydrated, with only 10% draining through gravity and the remaining 90% more slowly through evaporation. BC is stable at high temperatures, with thermal decomposition only occurring at temperatures above 365° C. 86. BC is a remarkable biomaterial, however to the synthetic biologist; BC is additionally interesting. Since BC is produced by bacteria, and synthetic biology has spent around 20 years creating tools that control bacteria, BC could provide the perfect base from which we could craft a range of engineered living materials.

There is a need for additional and advanced tools and methods that can be used to create bacterial cellulose with particular properties to make them more suitable for use in a real-world context.

The present invention addresses this need by providing methods and components for producing self-pigmented bacterial cellulose, methods of spatially restricted gene expression, spatially pigmented bacterial cellulose, amongst other advantageous products and methods.

### BRIEF DESCRIPTION OF THE INVENTION

The inventors have surprisingly discovered that a two-step process is needed to achieve melanated bacterial cellulose. The inventors have shown that melanin formation is inhibited by the low pH conditions of that result from the necessary culture of the bacteria to form the cellulose pellicle and so pellicle formation has to be temporally separated from melanin formation.

The inventors have also generated a sophisticated means to produce bacterial cellulose that comprises spatially restricted gene expression, and which can be used to, for example, form spatially restricted pigmented bacterial cellulose.

### DETAILED DESCRIPTION OF THE INVENTION

Bacterial cellulose is produced by many bacteria. The present inventors have found that it is possible to express tyrosinase in these bacteria, with tyrosinase being a key enzyme in the formation of melanin. However, unexpectedly, the inventors found that whilst the bacteria could express the tyrosinase enzyme, melanin was not produced during bacterial culture and formation of the pellicle. It was only when the pellicle was separately processed in a relatively neutral pH development solution was melanin produced and the cellulose changed colour. Without wishing to be bound by any theory, it appears that the low pH that arises as a result of the culture of particular bacterial cellulose producing bacteria inhibits the formation of melanin.

Accordingly, in a first aspect, the invention provides a method for producing melanated bacterial cellulose, wherein the method comprises exposing a cellulose pellicle that comprises tyrosinase to a development solution, wherein the development solution is at a pH of between 6 and 8.5.

It is considered that increasing the pH from the low pH arising from the growth of the bacteria is a key determinant in the formation of melanin.

However, additional factors are considered to be necessary for function of the tyrosinase enzyme and the formation of melanin. For example, in some embodiments the invention provides a method for producing melanated bacterial cellulose, wherein the method comprises exposing a cellulose pellicle that comprises tyrosinase to a development solution, wherein the development solution:
is at a pH of between 6 and 8.5; and
comprises L-tyrosine and/or L-cysteine and/or L-cystine.

In some instances the development solution also comprises metal ions with an oxidation state of 2+.

In some embodiments it is considered that the bacterial cellulose already comprises sufficient metal ions and L-tyrosine and/or L-cysteine and/or L-cystine for melanin formation.

In some embodiments, the metal ions are required to act as co-factors for the tyrosinase enzyme. In some embodiments the development solution is capable of resulting in the development of melanin without the addition of metal ions. In some embodiments the development solution is capable of resulting in enhanced development of melanin, or a faster development of melanin, when metal ions are added to the development solution.

Accordingly in one embodiment the development solution:
is at a pH of between 6 and 8.5; and
a) comprises metal ions capable of acting as a co-factor for tyrosinase; and/or
b) comprises L-tyrosine and/or L-cysteine and/or L-cystine.

In a preferred embodiment the development solution:
is at a pH of between 6 and 8.5; and
comprises metal ions capable of acting as a co-factor for tyrosinase; and
comprises L-tyrosine and/or L-cysteine and/or L-cystine.

In preferred embodiments the bacterial cellulose pellicle was produced by bacterial cells that express tyrosinase.

In some embodiment, the method for producing melanated bacterial cellulose includes a step of culturing a cellulose producing bacteria under conditions so as to allow a pellicle to form. For example, in one embodiment the invention provides:
a method for producing melanated bacterial cellulose, wherein the method comprises:
   a) culturing a cellulose producing bacteria under conditions so as to allow a pellicle to form, wherein the bacteria express tyrosinase; and
   b) exposing the pellicle formed in (a) to a development solution;
wherein the development solution:
is at a pH of between 6 and 8.5; and
a) comprises metal ions capable of acting as a co-factor for tyrosinase; and/or
b) comprises L-tyrosine and/or L-cysteine and/or L-cystine.

The skilled person will be aware of the culture conditions that are necessary to allow the bacteria to grow and form a pellicle. In some embodiments the conditions that allow a pellicle to form comprise culturing the bacteria:
a) at a pH of:
   between 3-7, optionally a pH of between 3.25 and 6.75, 3.5 and 6.5, 3.5 and 6.25, 3.75 and 6, 4 and 5.75, 4.25 and 5.5, 4.5 and 5.25; pH 5.8; and/or
   at least 3 but less than or equal to pH 7, for example at least 3, 3.25, 3.5, 3.75, 4, 4.25, 4.5, 4.75, 5, 5.25, 5.5, 5.75, 5.8, 6, 6.25, 6.5, 6.75, but less than or equal to pH 7;
      and/or
b) in culture media that is:
   i) HS media; or
   ii) Coconut water media.

Coconut water media is considered to comprise coconut water and 1% vinegar.

In preferred embodiments, step (b) of exposing the pellicle to a development solution is performed after the pellicle formed in (a) is harvested. By harvested we include the meaning of removing the pellicle from the culture which produced the pellicle. In some embodiments harvesting also includes washing the pellicle in water or PBS for example.

The pellicle may comprise any tyrosinase enzyme; and the bacterial that produced the pellicle may express any tyrosinase enzyme.

However, in preferred embodiments the tyrosinase is a bacterial tyrosinase. In particular embodiments the bacterial tyrosinase is selected from the group comprising:
i) Tyr1 from Bacillus megaterium [SEQ ID NO: 13];
li) mel from Streptomyces antibiotics [SEQ ID NO: 14]; or
iii) mel from Rhizobium etli [SEQ ID NO: 15];
   optionally wherein the tyrosinase comprises or consists of a sequence with at least 80%, 82%, 84%, 86%, 88%, 90%, 92%, 94%, 96%, 98%, 99% or 100% sequence identity to any of the above sequences.

In some embodiments the tyrosinase is heterologous, i.e. the tyrosinase is not a tyrosinase that is normally expressed by the particular bacteria.

It will be apparent that the bacterial cells that produce the bacterial cellulose are capable of producing bacterial cellulose. The Komagataeibacter cellulose synthase operon is illustrated in FIG. 2. The genes bcsA, bcsB, bcsC are found across all cellulose synthase operons, whilst bcsD is unique to Komagataeibacter. The genes bcsA and bcsB can also been found fused together into a single gene bcsAB. Interestingly, both bcsAB and bcsA:bcsB forms can be found in the same genome due to duplication of the cellulose synthase operon, a phenomenon that is observed in many Komagataeibacter. Alongside these core genes, the Komagataeibacter cellulose synthase operon also contains the flanking genes cmcAx, ccpAx and bglAx.

In some embodiments, bacteria that are capable of producing bacterial cellulose are bacteria that express all of the following genes:
bcsA, bcsD, bscC and bscD.

One embodiment of the method of the invention described herein wherein the melanin is produced subsequent to formation of the pellicle is considered to be particularly advantages for use with bacterial pellicles that have been produced by bacteria that lower the pH during culture. For example, Acetobacter and Komagataeibacter produce bacterial cellulose and lower the pH during growth. For such bacteria, the low pH prevents the formation of melanin during culture, and it is only when the pH is raised during the subsequent “developing” step that the tyrosinase is able to catalyse the formation of melanin.

In some embodiments, the bacteria that is cultured, or the bacteria that produced the cellulose pellicle is selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes.

In preferred embodiments the bacteria is from the genus Komagataeibacter. Species of this genus are particular over-producers of bacterial cellulose. The Komagataeibacter genus includes at least the species indicated in FIG. 1, though it is expected that other species may be added in future as new species are discovered.

In some embodiments, the bacteria that is cultured, or the bacteria that produced the cellulose pellicle is selected from the group comprising or consisting of:

Komagaelbacter rhaeticus; Komagaelbacter xylinus, Komagaelbacter hansenii, Komagaelbacter medellinensis, Komagaelbacter europaeus, Komagaeibacter maltaceti, Komagaelbacter pomaceti, Komagaeibacter oboediens, or Komagaeibacter saccharivoans.

In further embodiments, the bacterial cells are selected from the group comprising or consisting of:
a) a strain of Komagaelbacter rhaeticus selected from the group comprising or consisting of: Komagaelbacter rhaeticus IGEM. Komagaeibacter rhaeticus AF1; Komagaeibacter rhaeticus LMG22126; or
b) Gluconacetobacter xylinus CGMCC 2995.

In a preferred embodiment, the bacterial cells are Komagaelbacter rhaeticus iGEM cells.

As described above, the cellulose pellicle is exposed to a “developing solution”.

A key factor in allowing the formation of melanin to take place is an adjustment of the pH from that of the culture that the pellicle was grown in, to a higher pH which allows tyrosinase to catalyse the formation of melanin.

Accordingly in some embodiments the development solution is at a pH of:
at least 6, optionally at least 6.25, 6.5, 6.75, 7, 7.25, 7.4, 7.5, 7.75, 8, 8.25, or at least 8.5; and/or
between 6 and 8.5, optionally between 6.25 and 8.25, 6.5 and 8, 6.25 and 7.75, 6.5 and 7.5, 6.75

In preferred embodiments the pH of the development solution is pH 7.4.

In addition to an appropriate pH, tyrosinase requires metal co-factors and tyrosine to produce melanin. In some embodiments sufficient metal co-factors and tyrosine may be present in the culture medium in which the pellicle was grown.

Accordingly in some embodiments the development solution comprises components that can be added to the bacterial culture to increase the pH into the ranges described above.

However, in preferred embodiments, the pellicle is removed from the bacterial culture i.e., harvested, and then exposed to the development solution at the appropriate pH.

In some embodiments then, the development solution comprises:
metal ions capable of acting as a co-factor for tyrosinase;
   and
L-tyrosine and/or L-cysteine and/or L-cystine.

In some embodiments the metal ions capable of acting as a co-factor for tyrosinase are metal ions with an oxidation state of 2+.

In some embodiments the metal ions with an oxidation state of 2+ are selected from:
a) Cu²⁺, Zn²⁺, Be²⁺, Mg²⁺, Ca²⁺, Cr²⁺, Mn²⁺, Co²⁺ or Ni²⁺;
b) Cu²⁺, Zn²⁺; or
c) Cu²⁺.

It is considered that any means may be employed so as to result in the formation of metal ions that are capable of acting as a co-factor for tyrosinase, for example metal ions with an oxidation state of 2+—for example Cu1+ salts may be used which would be expected to oxidise to form Cu²⁺ capable of acting as a co-factor.

In some embodiments the development solution comprises a water-soluble copper (II) salt, optionally comprises CuSO₄, or CuCl₂ optionally comprises:
A) at least 2 μM CuSO₄, optionally at least 3 μM, 4 μM, 5 μM, 7.5 μM, 10 μM,
12.5 μM, 15 μM, 17.5 μM, 20 μM, 40 μM, 60 μM, 80 μM, 100 μM, 120 μM, 140 μM, or at least 160 μM CuSO₄; and/or
   between 2 μM CuSO₄ and 20 μM CuSO₄; and/or
   less than 20 μM, optionally less than 17.5 μM, 15 μM, 12.5 μM, 10 μM, 7.5 μM, 5 μM, 4 μM, 3 μM, or less than 2 μM; and/or
   between 20 μM and 160 μM CuSO₄; and/or
   less than 160 μM, optionally less than 140 μM, 120 μM, 100 μM, 80 μM, 60 μM, 40 μM, or less than 20 μM CuSO₄;
      and/or
B) at least 2 μM CuCl₂, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuCl₂; and/or
   between 2 μM CuCl₂ and 20 μM CuCl₂; and/or
   less than 20 μM, optionally less than 17.5 μM, 15 μM, 12.5 μM, 10 μM, 7.5 μM, 5 μM, 4 μM, 3 μM, or less than 2 μM CuCl₂; and/or
   between 20 μM and 160 μM CuCl₂; and/or
   less than 160 μM, optionally less than 140 μM, 120 μM, 100 μM, 80 μM, 60 μM, 40 μM, or less than 20 μM CuCl₂.

The skilled person will appreciate that tyrosinase catalyses the formation of eumelanin from tyrosine but can also catalyse the formation of pheomelanin from cysteine or cystine. Accordingly, the type of melanin, and therefore the colour of bacterial cellulose that is produced depend on whether the development solution comprises tyrosine, or cysteine/cystine. The skilled person will be able to determine appropriate concentrations of tyrosine or cysteine/cystine to expose the pellicle to.

In some embodiments the development solution comprises:
at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L tyrosine; and/or
less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine.

In some embodiments the development solution comprises:
A) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
   between 10 g/L and 300 g/L cysteine; and/or
   less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
      and/or
B) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L;
   between 0.1 g/L and 2 g/L L-cystine; and/or
   less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine.

The skilled person will appreciate that the development solution must be appropriately buffered. Accordingly, in some embodiments the development solution comprises a buffer, for example a buffer selected from the group comprising PBS, HEPES, MOPS and TRIS, optionally wherein the buffer is PBS. In some embodiments, the development solution comprises PBS wherein the PBS is present at a concentration between 1× and 20×PBS, such 1×PBS, 2×PBS, 3×PBS, 4×PBS, 5×PBS, 6×PBS, 7× PBS, 8×PBS, 9×PBS, 10×PBS, 11×PBS, 12×PBS, 13×PBS, 14×PBS, 15×PBS, 16× PBS, 17×PBS, 18×PBS, 19×PBS, or 20×PBS. In a preferred embodiment, the PBS is present at a concentration of 1×. It will be appreciated, however, that the buffering requirements of the development solution may vary depending on the size of the pellicle that is exposed to the development solution. For example, it may be appropriate to buffer the development solution with a higher concentration of buffer when a larger pellicle is exposed to the development solution, than when a smaller pellicle is exposed to the development solution. The person skilled in the art may determine whether an increased concentration of buffer should be used, for example, by monitoring the pH of development solution during the development reaction. If, for example, the pH of the development solution decreases below pH 7 during the development reaction, it may be appropriate to use a higher concentration of buffer; or to add additional buffer to increase the buffer concentrations.

In particular embodiments the development solution comprises:
a) PBS at pH 7.4;
b) 10 μM CuSO₄, or 20 μM CuSO₄; and
c) 0.5 g/L L-tyrosine or 1 g/L L-tyrosine, and/or 1 g/L L-cysteine, and/or 0.4 g/L L-cystine.

As described above, the bacterial pellicle that comprises tyrosinase is exposed to the development solution. The duration of solution can be any duration and can be chosen so as to produce the required amount of melanin. i.e., is a very strongly pigmented cellulose is required, the pellicle should be exposed to the development solution for a longer duration than if a less strongly pigmented cellulose is required.

In some embodiments, the pellicle is exposed to the development solution for:
at least 1 hour, optionally at least 2 hours, 3, 4, 5, 6, 7, 8, 9, 10, 11, 12, 13, 14, 15, 16, 17, 18, 19, 20, 21, 22, 23, 24, 36, or at least 48 hours;
between 1 hour and 48 hours, or between 2 and 36, 3 and 24, 4 and 23, 5 and 22, 6 and 21, 7 and 20, 8 and 19, 9 and 18, 10 and 17, 11 and 16, 12 and 15, 13 and 14 hours; and/or
for less than 48 hours, optionally less than 36 hours, 24, 23, 22, 21, 20, 19, 18, 17, 16, 15, 14, 13, 12, 11, 10, 9, 8, 7, 6, 5, 4, 3, 2, or less than 1 hour.

The temperature at which the pellicle is incubated in the development solution can also affect how pigmented the cellulose becomes. Reactions at a higher temperature tend to occur faster than at a lower temperature, and the skilled person can use duration of exposure, temperature of exposure, or a combination of both duration and temperature of exposure to produce melanated cellulose that is melanated to the desired amount.

In some embodiments the cellulose pellicle is incubated in the development solution at a temperature of:
between 25° C. and 50° C., optionally between 30° C. and 45° C., 35° C. and 40° C.; and/or
at least 25° C. optionally at least 30° C., 32° C., 34° C., 36° C., 38° C., 40° C., 42° C., 44° C., 46° C., 48° C. or at least 50° C.; or
30° C.; or
45° C.

It will be appreciated that once the melanin has formed in the cellulose, it may be desirable to sterilise the pellicle material prior to further processing or use as a textile for example.

The inventors have surprisingly found that particular types of sterilisation, such as oxidation-based methods such as bleach and hydrogen peroxide; and alkali or acidic conditions are not the most appropriate since the colour is bleached out, or leaches out of the pellicle.

Accordingly in some embodiments the pellicle is not sterilised using bleach, hydrogen peroxide, acidic conditions or alkalic conditions.

In preferred embodiments the methods of the invention comprises the further step of:
(c) sterilising the pellicle following incubation in the development solution.

In some embodiments the sterilisation is selected from the group comprising or consisting of:
i) autoclaving;
ii) heating; and/or
iii) desiccation, optionally with 70% ethanol.

As described above, in preferred embodiments the tyrosinase is a bacterial tyrosinase, for example:
Tyr1 from Bacillus megaterium [SEQ ID NO: 13];
mel from Streptomyces antibiotics [SEQ ID NO: 14]; or
mel from Rhizobium etli [SEQ ID NO: 15];

In some embodiments the tyrosinase comprises or consists of a sequence with at least 80%, 82%, 84%, 86%, 88%, 90%, 92%, 94%, 96%, 98%, 99% or 100% sequence identity to any of the above sequences.

In some embodiments the tyrosinase is a heterologous tyrosinase.

In some embodiments, particularly where the bacteria is a bacteria of the Komagaelbacter genus, for example where the bacteria is Komagaelbacter rhaeticus IGEM, the tyrosinase gene is operably linked to Anderson promoter J23104 [SEQ ID NO: 16] and RBS B0034 [SEQ ID NO: 17]. In some embodiments the tyrosinase gene is operably linked to Anderson promoter J23104 and RBS B0034 that comprises or consists of a sequence with at least 80%, 82%, 84%, 86%, 88%, 90%, 92%, 94%, 96%, 98%, 99% or 100% sequence identity to any of the above sequences.

The culture medium used to culture the bacteria as they produce the cellulose pellicle can be any appropriate culture media. The skilled person will be well aware of a range of suitable culture media.

In some embodiments the cells are cultured in a culture medium that:
i) is Hestrin and Schramm (HS) medium;
ii) is supplemented with glucose, optionally at 2% (w/v); and/or
iii) is buffered to a pH of 5.8.

In addition to the methods described above, the invention also provides a nucleic acid comprising a regulatory sequence and a sequence that encodes a tyrosinase enzyme wherein the regulatory sequence comprises Anderson promoter J23104 and RBS B0034. In some embodiments the nucleic acid also comprises a terminator sequence.

In some embodiments the sequence that encodes a tyrosinase enzyme encodes a bacterial tyrosinase, for example encodes:
Tyr1 from Bacillus megaterium [SEQ ID NO: 13];
mel from Streptomyces antibiotics [SEQ ID NO: 14]; or
mel from Rhizobium etli [SEQ ID NO: 15];
   for example, in some embodiments the tyrosinase comprises or consists of a sequence with at least 80%, 82%, 84%, 86%, 88%, 90%, 92%, 94%, 96%, 98%, 99% or 100% sequence identity to any of the above sequences.

The nucleic acid may be any nucleic acid, but in some embodiments the nucleic acid is a circular nucleic acid, for example is a circular nucleic acid selected from the group consisting of a plasmid, a bacterial artificial chromosome, a phagemid, a cosmid, a yeast artificial chromosome, a human artificial chromosome, a viral vector.

In preferred embodiments the circular nucleic acid further comprises an origin of replication.

In preferred embodiments the circular nucleic acid further comprises a selectable marker.

In some embodiments the nucleic acid that encodes the tyrosinase enzyme is integrated into the genome of a cell, optionally a bacterial cell.

In addition to the nucleic acid that encodes the tyrosinase enzyme, the invention also provides a cell that comprises the nucleic acid. Accordingly in some embodiments the invention provides:
cell comprising the nucleic acid of the invention, for example wherein the cell is:
   i) a bacterial cell that is capable of producing bacterial cellulose;
   ii) a bacterial cell that expresses all of bcsA, bcsD, bscC and bscD;
   iii) a bacterial cell of a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes;
   iv) a bacterial cell selected from the group comprising or consisting of: Komagaeibacter rhaeticus; Komagaeibacter xylinus, Komagaeibacter hansenii, Komagaeibacter medellinensis, Komagaeibacter europaeus, Komagaelbacter maltaceti, Komagaelbacter pomaceti, Komagaelbacter oboediens, or Komagaelbacter saccharivoans;
   v) a bacterial cell selected from the group comprising or consisting of:
      a) a strain of Komagaeibacter rhaeticus selected from the group comprising or consisting of:
         Komagaeibacter rhaeticus iGEM; Komagaeibacter rhaeticus AF1; Komagaeibacter rhaeticus LMG22126; or
      b) Gluconacetobacter xylinus CGMCC 2995;
   vi) a bacterial cell that is a Komagaeibacter rhaeticus IGEM cell.

The above cells are all cells that are considered to be useful for the production of bacterial cellulose. However, the skilled person will also appreciate that the nucleic acid of the invention may be maintained, for example for production of cloning purposes, in non-cellulose producing cells. Accordingly, the invention provides cells, for example non-cellulose producing bacterial cells, that comprise the nucleic acid of the invention.

Described above is a means of producing melanated bacterial cellulose which require a development step that is separate to the formation of the pellicle.

The position of melanin pigment depends on where in the pellicle the tyrosinase enzyme is. The inventors have also devised a system for use in bacteria of the genus Komagataeibacter which allows for the spatially restricted expression of a gene which can be used to produce spatially restricted pigmented cellulose.

The fact that expression of the necessary genes and proteins is restricted locally to particular cells was not expected—it was possible that the necessary enzymes become diffuse in the pellicle so that upon exposure to the development solution the entire pellicle becomes pigmented. Surprisingly, this is not the case, and as the examples demonstrate it is possible to produce highly targeted regions of pigmented cellulose in an otherwise unpigmented cellulose pellicle.

The method relies on the development of an optogenetic expression system in Komagataeibacter that is used to direct the expression of a target protein or RNA, either during pellicle growth, or following growth of the pellicle to the desired size/thickness.

The optogenetic expression system is made suitable for light-induced expression in Komagataeibacter by using split proteins that are only re-joined and made functional upon exposure to light.

One method is to place the gene of interest (third nucleic acid sequence as described herein) under the control of a promoter that is only operated by a particular, heterologous polymerase. For example, in some embodiments described herein, the gene of interest is under the control of the T7 promoter. The gene encoding the T7 polymerase is split into two fusion proteins, each part of the T7 polymerase being fused to a dimerization domain that dimerises only in the presence of light of a particular wavelength or range of wavelengths. Accordingly, upon exposure of the system to light of the dimerization wavelength, the dimerization domains dimerise bringing the two portions of the T7 polymerase back together so as to produce a functional polymerase than can transcribe the gene of interest.

In another similar embodiment, rather than splitting a polymerase, a particular transcription factor, such as LuxR, is split and fused to dimerization domains. The gene of interest (third nucleic acid sequence as described herein) is under the control of a promoter that is only activated in the presence of the functional transcription factor. In a similar way, exposure of the system to the dimerization wavelength causes dimerization and reformation of a functional transcription factor and subsequence transcription of the gene of interest. Using a split-transcription factor with a light-inducible dimerization domain has been shown to function in E. coli and it is expected that a similar approach would operate in Komagataeibacter (Romano et al Nature Chemical Biology 17: 817-827).

The skilled person will recognise that there are many combinations that can be employed to achieve the light-induced expression of a gene of interest using the disclosure herein.

Accordingly, in one embodiment the invention provides:
an optogenetic expression system for use in bacteria of the genus Komagataeibacter, comprising:
   (a) A first nucleic acid comprising a first nucleotide sequence that encodes a first polypeptide, wherein the first polypeptide comprises:
      i) a first domain that comprises a first portion of a heterologous split-polymerase or a split-transcription factor; and
      ii) a second domain that comprises a first light-inducible dimerization domain;
   (b) A second nucleic acid comprising a second nucleotide sequence that encodes a second polypeptide, wherein the second polypeptide comprises:
      i) A first domain that comprises a second portion of a heterologous split-polymerase or a split-transcription factor; and
      ii) A second domain that comprises a second light-inducible dimerization domain;
and
   (c) A third nucleic acid comprising a third nucleic acid sequence that encodes a target protein or RNA to be expressed operably linked to a target promoter;
and wherein the first light-Inducible dimerization domain and the second light-inducible dimerization domain are capable of dimerising with one another upon exposure to light of a dimerization wavelength to form a functional heterologous polymerase or a functional transcription factor capable of transcribing or initiating transcription from the target promoter,
and wherein the target promoter is recognised by the functional heterologous polymerase or functional transcription factor so as to drive transcription of the third nucleic acid sequence that encodes a target protein or RNA.

In some embodiments where the first polypeptide comprises a first domain that comprises a portion of a heterologous split-polymerase and the second polypeptide comprises a first domain that comprises a second portion of the heterologous split-polymerase the optogenetic expression system is a polymerase-based optogenetic system.

In some embodiments where the first polypeptide comprises a first domain that comprises a portion of a split-transcription factor and the second polypeptide comprises a first domain that comprises a second portion of the split-transcription factor the optogenetic expression system is a transcription factor-based optogenetic system.

In some embodiments the target promoter is a heterologous promoter, i.e., is a promoter not typically found in the wild-type version of the bacteria, i.e., the promoter is not native to the bacterial strain or species.

The skilled person will appreciate that although the optogenetic expression system comprises three nucleic acids, each comprised of different parts, the three nucleic acids may actually be present on the same nucleic acid molecule or may be separate individual nucleic acid molecules.

For example, in one embodiment of the optogenetic expression system the first nucleic acid, the second nucleic acid and the third nucleic acid are all part of the same nucleic acid molecule.

The nucleic acid molecule that comprises each of the three nucleic acids of the expression system may be any nucleic acid, but in some embodiments the nucleic acid is a circular nucleic acid, for example is a circular nucleic acid selected from the group consisting of a plasmid, a bacterial artificial chromosome, a phagemid, a cosmid, a yeast artificial chromosome, a human artificial chromosome, a viral vector.

Combining all of the nucleic acids onto a single nucleic acid molecule such as a circular nucleic acid has advantages in that only one selectable marker for instance is needed, and it not necessary to maintain several vectors within the bacteria.

In preferred embodiments the circular nucleic acid further comprises an origin of replication.

In preferred embodiments the circular nucleic acid further comprises a selectable marker.

In other embodiments, the three nucleic acids of the optogenetic expression system are different separate nucleic acid molecules.

In some embodiments any two of the three nucleic acids of the optogenetic system are on the same nucleic acid molecule, and the third nucleic acid is a separate nucleic acid molecule.

In some embodiments the three nucleic acids of the optogenetic expression system are integrated into the genome of a cell, optionally a bacterial cell. In this way, the nucleic acids required for the optogenetic expression system are stably maintained.

In some embodiments, the first and second nucleic acids of the optogenetic expression system are integrated into the genome a cell, optionally a bacterial cell; and the third nucleic acid of the optogenetic expression system is maintained episomally in the cell, optionally a bacterial cell. In this way, the first and second nucleic acids required for the optogenetic expression system are stably maintained; and the third nucleic acid required for the optogenetic expression system is interchangeable. In embodiments where the first and second nucleic acids of the optogenetic expression system are integrated into the genome the cell, the fourth nucleic acid may optionally also be integrated into the chromosome of said cell.

The skilled person is well able to integrate nucleic acids into the genome of a cell, for example the genome of a bacterial cell.

As described above, in some embodiments the optogenetic expression system acts through a split-polymerase system, and in other embodiments it operates through a split-transcription factor system.

In some embodiments where the system acts through a heterologous split-polymerase system, the heterologous split-polymerase is a split-T7 polymerase.

The skilled person will be aware that the T7 polymerase can be “split” in a number of places but still result in the formation of a functional T7 polymerase activity when the two portions are brought back together. See for example Baumschlager et al ACS Synthetic Biology 2017 6: 2157-2167. Exemplary sequences for the first portion and the second portion of the split T7 polymerase are given in SEQ ID NO: 1 and 2. Further sequences for the first and second portion of the split T7 polymerase as provided herein are given in SEQ ID NO: 27, SEQ ID NO: 33, and SEQ ID NO: 35.

Accordingly, in some embodiments the functional heterologous polymerase that is formed once the dimerization domains dimerise upon exposure to light of the dimerization wavelength is a T7 polymerase.

In some embodiments then:
the first portion of the heterologous split-polymerase comprises or consists of a sequence of at least 70%, at least 75%, at least 80%, at least 85%, at least 90%, at least 95%, at least 96%, at least 97%, at least 98%, or at least 99% identical to SEQ ID NO: 1 and/or SEQ ID NO: 27 or 100% identical to SEQ ID NO: 1 and/or SEQ ID NO: 27; and/or
the second portion of the heterologous split-polymerase comprises or consists of a sequence of at least 70%, at least 75%, at least 80%, at least 85%, at least 90%, at least 95%, at least 96%, at least 97%, at least 98%, or at least 99% Identical to SEQ ID NO: 2, SEQ ID NO: 33, and/or SEQ ID NO: 35 or 100% identical to SEQ ID NO: 2, SEQ ID NO: 33, and/or SEQ ID NO: 35.

In some embodiments, the first portion of the heterologous split-polymerase and the second portion of the heterologous split-polymerase comprises or consists of a pair of sequences selected from:
SEQ ID NO: 1 and SEQ ID NO: 2;
SEQ ID NO: 27 and SEQ ID NO: 33; or
SEQ ID NO: 1 and SEQ ID NO: 35;
or sequences that are at least 70%, at least 75%, at least 80%, at least 85%, at least 90%, at least 95%, at least 96%, at least 97%, at least 98%, or at least 99% Identical thereto, or 100% identical thereto.

The first portion of the split-polymerase is in some embodiments encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 3, SEQ ID NO: 26, or 100% identical to SEQ ID NO: 3 and/or SEQ ID NO: 26; and/or

the second portion of the heterologous split-polymerase is encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 4, SEQ ID NO: 32, and/or SEQ ID NO: 34, or 100% Identical to SEQ ID NO: 4, SEQ ID NO: 32, and/or SEQ ID NO: 34.

In some embodiments, the first portion of the heterologous split-polymerase and the second portion of the heterologous split-polymerase are encoded by a DNA sequence that comprises or consists of a pair of sequences selected from:
SEQ ID NO: 3 and SEQ ID NO: 4;
SEQ ID NO: 26 and SEQ ID NO: 32; or
SEQ ID NO: 3 and SEQ ID NO: 34;
or sequences that are at least 70%, at least 75%, at least 80%, at least 85%, at least 90%, at least 95%, at least 96%, at least 97%, at least 98%, or at least 99% identical thereto, or 100% identical thereto.

In some embodiments where the system acts through split-transcription factor mechanisms, the split-transcription factor is LuxR, and the target promoter comprises a LuxR binding site.

The skilled person will appreciate that:
a) the first nucleic acid comprises a first promoter operably linked so as to drive expression of the first polypeptide; and/or
b) the second nucleic acid comprises a second promoter operably linked so as to drive expression of the second polypeptide.

The light-inducible dimerization domain can be any domain that is capable of forming a homodimer or heterodimer upon exposure to light of a particular wavelength. There are numerous examples of such domains and the skilled person will be well aware of these—see for example Leopol et al Chem Soc Rev 2018 47: 2454-2484.

Exemplary combinations of domains include:
the first light-inducible dimerization domain is a LOV dimerization domain and the second light-inducible dimerisation domain is a LOV dimerisation domain; the first light-inducible dimerization domain is an nMag dimerization domain and the second light-inducible dimerisation domain is a pMag dimerisation domain;
the first light-inducible dimerization domain is a pMag dimerization domain and the second light-inducible dimerisation domain is an nMag dimerisation domain; the first light-inducible dimerization domain is a VVD dimerization domain and the second light-inducible dimerization domain is a VVD dimerization domain;
the first light-inducible dimerization domain is a LOVtrap dimerization domain and the second light-inducible dimerisation domain is an LOVtrap dimerisation domain;
the first light-inducible dimerization domain is a VfAU1-LOV dimerization domain and the second light-inducible dimerisation domain is a VfAU1-LOV dimerisation domain;
the first light-inducible dimerization domain is a NgPA1-LOV dimerization domain and the second light-inducible dimerisation domain is a NgPA1-LOV dimerisation domain;
the first light-inducible dimerization domain is a OdPA1-LOV dimerization domain and the second light-inducible dimerisation domain is a OdPA1-LOV dimerisation domain;
the first light-inducible dimerization domain is a AsLOV2 dimerization domain and the second light-inducible dimerisation domain is an PDZ dimerisation domain;
the first light-inducible dimerization domain is a PDZ dimerization domain and the second light-inducible dimerisation domain is a AsLOV2 dimerisation domain;
the first light-inducible dimerization domain is a AtCry2 dimerization domain and the second light-inducible dimerisation domain is a AtCry2 dimerisation domain;
the first light-inducible dimerization domain is a PhyB dimerization domain and the second light-inducible dimerisation domain is a PIF dimerisation domain;
the first light-inducible dimerization domain is a PIF dimerization domain and the second light-inducible dimerisation domain is a PhyB dimerisation domain;
the first light-inducible dimerization domain is a Cph1 dimerization domain and the second light-inducible dimerisation domain is a Cph1 dimerisation domain;
the first light-inducible dimerization domain is a CBD dimerization domain and the second light-inducible dimerisation domain is a CBD dimerisation domain.

In some embodiments the dimerization domains are magnet proteins, for example are the nMag and pMag proteins. Accordingly, in some embodiments the first light-Inducible dimerization domain is nMag and the second light-inducible dimerization domain is pMag; or the first light-inducible dimerization domain is pMag and the second light-inducible dimerization domain is nMag.

In some embodiments the nMag dimerization domain comprises or consists of a sequence that is at least 70%, at least 75%, at least 80%, at least 85%, at least 90%, at least 95%, at least 96%, at least 97%, at least 98%, or at least 99% identical to SEQ ID NO: 5 or 100% identical to SEQ ID NO: 5; and/or the pMag dimerization domain comprises or consists of a sequence that is at least 70%, at least 75%, at least 80%, at least 85%, at least 90%, at least 95%, at least 96%, at least 97%, at least 98%, or at least 99% identical to SEQ ID NO: 6, SEQ ID NO: 31, and/or SEQ ID NO: 45 or 100% Identical to SEQ ID NO: 6, SEQ ID NO: 31, and/or SEQ ID NO: 45.

In some embodiments the nMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% identical to SEQ ID NO: 7 or 100% identical to SEQ ID NO: 7 and/or the pMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% Identical to SEQ ID NO: 8, SEQ ID NO: 30, and/or SEQ ID NO: 44 or 100% Identical to SEQ ID NO: 8, SEQ ID NO: 30, and/or SEQ ID NO: 44.

In some embodiments then the split-polymerase is a T7 split polymerase, and the light-induced dimerization domains are the pMag and nMag domains. Accordingly in some embodiments:
A) the first nucleic acid sequence comprises or consists of a nucleotide sequence having at least 70%, at least 75%, at least 80%, at least 85%, at least 90%, at least 95%, at least 96%, at least 97%, at least 98%, or at least 99% identical to any of SEQ ID NO: 9 and SEQ ID NO: 28; and
   the second nucleic acid sequence comprises or consists of a nucleotide sequence having at least 70%, at least 75%, at least 80%, at least 85%, at least 90%, at least 95%, at least 96%, at least 97%, at least 98%, or at least 99% identical to any of SEQ ID NO: 12, SEQ ID NO: 36, SEQ ID NO: 38, SEQ ID NO: 40, and/or SEQ ID NO: 42; or
B) the first nucleic acid sequence comprises or consists of a nucleotide sequence having at least 70%, at least 75%, at least 80%, at least 85%, at least 90%, at least 95%, at least 96%, at least 97%, at least 98%, or at least 99% Identical to any of SEQ ID NO: 10; and
   the second nucleic acid sequence comprises or consists of a nucleotide sequence having at least 70%, at least 75%, at least 80%, at least 85%, at least 90%, at least 95%, at least 96%, at least 97%, at least 98%, or at least 99% identical to any of SEQ ID NO: 11.

In some embodiments, the first nucleotide sequence and the second nucleotide sequence comprise or consist of a pair of sequences selected from:
SEQ ID NO: 9 and SEQ ID NO: 12;
SEQ ID NO: 10 and SEQ ID NO: 11;
SEQ ID NO: 28 and SEQ ID NO: 38;
SEQ ID NO: 9 and SEQ ID NO: 36;
SEQ ID NO: 9 and SEQ ID NO: 40; or
SEQ ID NO: 9 and SEQ ID NO: 42
or sequences that are at least 70%, at least 75%, at least 80%, at least 85%, at least 90%, at least 95%, at least 96%, at least 97%, at least 98%, or at least 99% identical thereto, or 100% Identical thereto.

The skilled person will be aware that preferably in the absence of light of the dimerization wavelength, the first and second dimerization domains are substantially incapable of dimerising. Dimerization in the absence of light of the correct wavelength can lead to leakiness and some degree of expression of the third nucleic acid sequence that encodes a target protein or RNA to be expressed. The skilled person will be able to assess the level of product of the third nucleic acid sequence that is produced in the absence of light of the dimerization wavelength to determine if any basal level of transcription is acceptable. The level of leakiness that is acceptable will vary depending on the application.

The dimerization wavelength will depend on the properties of the light-inducible dimerization domains that are being used. The skilled person is readily able to determine the appropriate dimerization wavelength to expose the pellicle to. For example where the dimerization domains are the nMag and pMag domains, the skilled person knows that an appropriate dimerization wavelength would be about 400 nm to 500 nm; optionally a wavelength of between 400 nm and 500 nm, optionally 450 nm.

Accordingly, in some embodiments where:
the first light-inducible dimerization domain is an nMag dimerization domain and the second light-inducible dimerisation domain is a pMag dimerisation domain; or
the first light-inducible dimerization domain is a pMag dimerization domain and the second light-inducible dimerisation domain is an nMag dimerisation domain;
   the light of a dimerization wavelength has a wavelength of about 400 nm to 500 nm; optionally a wavelength of between 400 nm and 500 nm, optionally 450 nm.

The third nucleic acid sequence is capable of being transcribed into RNA that is an mRNA, i.e. is a protein coding mRNA, or is transcribed into a functional RNA that has other properties, for example is a CRISPR guide RNA. Accordingly the optogenetic expression system of the invention can be used to spatially express any protein or RNA in a specific chosen portion of a bacterial cellulose pellicle. In some embodiments this results in the activation of CRISPR methods in certain portions of the pellicle.

It will be clear to the skilled person that a cell that comprises the optogenetic expression system may also comprise one or more other modifications, for example to express one or more other proteins or RNAs. For example in some embodiments the cell is also modified so as to express a Cas protein, for example Cas9.

Accordingly in some embodiments the third nucleotide sequence is capable of being transcribed into mRNA, for example wherein the mRNA is capable of being translated into a polypeptide.

Accordingly, in some embodiments the third nucleotide sequence encodes a polypeptide.

As described elsewhere herein, bacterially cellulose is capable of being pigmented, for example pigmented with melanin.

The present invention therefore provides a system and a method for the spatially defined pigmentation of bacterial cellulose. Accordingly in some embodiments the third nucleotide sequence encodes a polypeptide that:
a) is involved in the biosynthesis of a pigment visible to the naked eye or in the biosynthesis of a molecule that emits light; or
b) encodes a protein that emits light or is a pigment.

In some preferred embodiments, the polypeptide that is involved in the biosynthesis of a pigment visible to the naked eye is an enzyme necessary for the formation of melanin, for example in the formation of melanin selected from the group comprising eumelanin, pheomelanin, neuromelanin and allomelanin.

As described elsewhere herein, the tyrosinase enzyme is required for the production of melanin. Accordingly, in some embodiments the third nucleotide sequence encodes a tyrosinase, for example bacterial tyrosinase, for example:
Tyr1 from Bacillus megaterium [SEQ ID NO: 13];
mel from Streptomyces antibiotics [SEQ ID NO: 14]; or
mel from Rhizobium etli [SEQ ID NO: 15];
   or encodes a tyrosinase that comprises or consists of a sequence with at least 80%, 82%, 84%, 86%, 88%, 90%, 92%, 94%, 96%, 98%, 99% or 100% sequence identity to any of the above sequences.

In preferred embodiments, expression of the polypeptide that is involved in the biosynthesis of a pigment visible to the naked eye results in the formation of the pigment, for example results in the formation of melanin, for example melanin selected from the group comprising eumelanin, pheomelanin, neuromelanin and allomelanin, for example where the polypeptide is a tyrosinase, for example a bacterial tyrosinase, for example:
Tyr1 from Bacillus megaterium [SEQ ID NO: 13];
mel from Streptomyces antibiotics [SEQ ID NO: 14]; or
mel from Rhizobium etli [SEQ ID NO: 15];
   or a tyrosinase that comprises or consists of a sequence with at least 80%, 82%, 84%, 86%, 88%, 90%, 92%, 94%, 96%, 98%, 99% or 100% sequence identity to any of the above sequences.

In some embodiments then the melanin is eumelanin and the third nucleic acid encodes tyrosinase, for example a bacterial tyrosinase, for example:
Tyr1 from Bacillus megaterium [SEQ ID NO: 13];
mel from Streptomyces antibiotics [SEQ ID NO: 14]; or
mel from Rhizobium etli [SEQ ID NO: 15];
   or encodes the tyrosinase that comprises or consists of a sequence with at least 80%, 82%, 84%, 86%, 88%, 90%, 92%, 94%, 96%, 98%, 99% or 100% sequence identity to any of the above sequences.

In some embodiments the protein that emits light emits light of a second wavelength when exposed to light of a first wavelength. In some embodiments such a protein is a fluorescent protein, for example a fluorescent protein selected from the group comprising or consisting of:

mCherry, GFP, mScarlet, mRFP, cjBlue, gfasPurple, eforRed, spisPink.

As is apparent to the skilled person, the two portions of the split-polymerase or split-transcription factor that are each fused to a light inducible dimerization domain are expressed within a cell, and so accordingly each fusion protein is expressed by a particular promoter that is associated with the first and second nucleic acids.

These promoters may be constitutive or may be inducible. Or one of the promoters may be constitutive and one of the promoters may be inducible.

In some embodiments then:
i) the first nucleic acid comprises a first promoter operably linked so as to drive expression of the first polypeptide; and/or
ii) the second nucleic acid comprises a second promoter operably linked so as to drive expression of the second polypeptide; and
   the first promoter and/or the second promoter are inducible promoters; or
   the first promoter and/or the second promoter are constitutive promoters.

In some embodiments wherein one or more of the first or second promoter is an inducible promoter is may be necessary to co-express within the cell a particular protein for example a heterologous protein that is required for inducible expression from the first and/or second promoter, optionally wherein the heterologous protein is a transcriptional regulator.

Accordingly in some embodiments the optogenetic expression system further comprises a fourth nucleic acid sequence that encodes a heterologous protein required for inducible expression from the first and/or second promoter, optionally wherein the heterologous protein is a transcriptional regulator.

In some embodiments the first and/or second promoter is selected from the group comprising or consisting of:
P_{BAD} [SEQ ID NO: 18];
pLux [SEQ ID NO: 19];
pTet [SEQ ID NO: 20]; or
pLac [SEQ ID NO: 21];
   or the first and/or second promoter comprises or consists of a sequence with at least 80%, 82%, 84%, 86%, 88%, 90%, 92%, 94%, 96%, 98%, 99% or 100% sequence identity to any of the above sequences.

In some embodiments the first and second promoter are both inducible promoters, for example selected from the following inducible systems:
the pBAD promoter [SEQ ID NO: 18] induced by arabinose in the presence of the transcriptional regulator araC [SEQ ID NO: 22];
the pLux promoter [SEQ ID NO: 23] induced by Acyl Homoserine Lactone (AHL) in the presence of the transcriptional regulator LuxR [SEQ ID NO: 23];
the pTet promoter [SEQ ID NO: 20] induced by Anhydrotetracycline (ATc) in the presence of the transcriptional regulator TetR [SEQ ID NO: 24]; or the pLac promoter [SEQ ID NO: 21] induced by IPTG in the presence of the transcriptional regulator LacI [SEQ ID NO: 25];
   for example, wherein:
the first promoter is pBAD and the second promoter is pBAD;
the first promoter is pLUX and the second promoter is pLUX;
the first promoter is pTet and the second promoter is pTet;
the first promoter is pLac and the second promoter is pLac;
or wherein the promoter comprises or consists of a sequence with at least 80%, 82%, 84%, 86%, 88%, 90%, 92%, 94%, 96%, 98%, 99% or 100% sequence identity to [SEQ ID NO: 18], [SEQ ID NO: 19], [SEQ ID NO: 20], [SEQ ID NO: 21], and/or wherein the transcriptional regulator comprises or consists of a sequence with at least 80%, 82%, 84%, 86%, 88%, 90%, 92%, 94%, 96%, 98%, 99% or 100% sequence identity to [SEQ ID NO: 22], [SEQ ID NO: 23], [SEQ ID NO: 24], [SEQ ID NO: 25].

In some embodiments it is preferably if the first and second promoters are both inducible promoters that are induced by the same inducer.

In some embodiments, the first and/or second promoters are inducible promoters that are induced by arabinose, for example where both the first and second promoters are induced by arabinose. In some embodiments the first and second promoters comprise the P_{BAD} promoter sequence, or for example comprise or consist of [SEQ ID NO: 18] or a sequence that has at least 80%, 82%, 84%, 86%, 88%, 90%, 92%, 94%, 96%, 98%, 99% or 100% sequence identity to [SEQ ID NO: 18].

In embodiments where the first and/or second promoter is inducible by arabinose and/or comprises the pBAD promoter, the fourth nucleic acid sequence encodes a transcriptional regulator selected from the group comprising or consisting of:

araC [SEQ ID NO: 22] or a sequence with at least 80%, 82%, 84%, 86%, 88%, 90%, 92%, 94%, 96%, 98%, 99% or 100% sequence identity to [SEQ ID NO: 22].

In embodiments where the first and/or second promoter is inducible by arabinose and/or comprises the pLUX promoter, the fourth nucleic acid sequence encodes a transcriptional regulator selected from the group comprising or consisting of:

LuxR [SEQ ID NO: 23] or a sequence with at least 80%, 82%, 84%, 86%, 88%, 90%, 92%, 94%, 96%, 98%, 99% or 100% sequence identity to [SEQ ID NO: 23].

In embodiments where the first and/or second promoter is inducible by arabinose and/or comprises the pTET promoter, the fourth nucleic acid sequence encodes a transcriptional regulator selected from the group comprising or consisting of:

TetR [SEQ ID NO: 24] or a sequence with at least 80%, 82%, 84%, 86%, 88%, 90%, 92%, 94%, 96%, 98%, 99% or 100% sequence identity to [SEQ ID NO: 24].

In embodiments where the first and/or second promoter is inducible by arabinose and/or comprises the pLAC promoter, the fourth nucleic acid sequence encodes a transcriptional regulator selected from the group comprising or consisting of:

LacI [SEQ ID NO: 25] or a sequence with at least 80%, 82%, 84%, 86%, 88%, 90%, 92%, 94%, 96%, 98%, 99% or 100% sequence identity to [SEQ ID NO: 25].

It will be clear then that in some embodiments the fourth nucleic acid sequence encodes a heterologous protein required for inducible expression from the first and/or second promoter, optionally wherein the heterologous protein is a transcriptional regulator, for example is araC.

The nucleic acids of the optogenetic expression system may also comprise other parts that are standard parts of expression systems. For example, in some embodiments any of the first nucleotide sequence, the second nucleotide sequence, the third nucleotide sequence, and/or the fourth nucleotide sequence are operably linked to an enhancer sequence, a terminator sequence, a repressor sequence, an operator sequence and/or a sigma factor binding site.

In addition to the optogenetic expression system described herein, the invention also provides a cell that comprises the optogenetic expression system of the invention. Preferences for the cell are as described elsewhere herein.

For example, in one embodiment the invention provides a cell that comprises the optogenetic system of the invention, for example in some embodiments:
the cell is capable of producing bacterial cellulose;
the cell is a bacterial cell that lowers the pH of the media during culture, for example produces an acid for example acetic acids;
the cell is not capable of producing bacterial cellulose (for example wherein the cell is a cell used in production or maintenance of the component parts of the optogenetic expression system);
the cell is selected from the group comprising or consisting of: a bacterial cell, an archaeal cell, or a eukaryotic cell;
the cell is a bacterial cell, optionally a bacterial cell that expresses all of bcsA, bcsD, bscC and bscD;
the cell is a bacterial cell that the bacterial cell belongs to a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes;
the cell is a bacterial cell selected from the group comprising or consisting of: Komagaelbacter rhaeticus Komagaelbacter rhaeticus; Komagaeibacter xylinus, Komagaelbacter hansenii, Komagaelbacter medellinensis, Komagaelbacter europaeus, Komagaelbacter maltaceti, Komagaelbacter pomaceti, Komagaeibacter oboediens, or Komagaeibacter saccharivoans;
the cell is a bacterial cell that is:
   a) a strain of Komagaeibacter rhaeticus selected from the group comprising or consisting of:
   Komagaelbacter rhaeticus iGEM; Komagaelbacter rhaeticus AF1; Komagaeibacter rhaeticus LMG22126; or
   b) Gluconacetobacter xylinus CGMCC 2995;
   the cell is a Komagaeibacter rhaeticus iGEM cell;
and/or
the cell is capable of producing bacterial cellulose and lowers the pH of the media during culture, for example produces an acid for example acetic acid.

It will be clear from the above disclosure, that the optogenetic expression system can be used to produce spatially pigmented bacterial cellulose. Accordingly, the invention also provides a method of producing spatially pigmented bacterial cellulose, comprising the steps of:
(a) providing a culture of cells of the invention (i.e., cells that comprise the optogenetic expression system of the invention described herein) wherein the third nucleotide sequence encodes a polypeptide that:
   i) is involved in the biosynthesis of a pigment visible to the naked eye or in the biosynthesis of a molecule that emits light; or
   ii) encodes a protein that emits light or is a pigment;
(b) maintaining the culture of (a) under conditions that allow the production of a cellulose pellicle by the microorganism; and
(c) exposing a spatially defined region or regions of the cellulose pellicle to light of the dimerization wavelength so as to allow expression of the third polypeptide.

In some embodiments the cellulose pellicle in (b) Is allowed to develop to the final desired area and/or thickness prior to exposing the defined region or regions to light in step (c). In some embodiments once the pellicle in (b) has developed to the final desired area and/or thickness the pellicle is harvested prior to exposing the spatially defined region or regions to light of the dimerization wavelength in step (c).

In the same or alternative embodiments, the spatially defined regions of the cellulose pellicle are exposed to the light during step (b).

The Inventors have found that in some instances when the pellicle is exposed to light of the dimerization wavelength during culture of the pellicle, evaporation from the culture and concomitant decrease in volume affects the focus of the spatially defined pigmented regions. In some instances, this may not be important. However, where sharp, defined edges between pigmented and non-pigmented pellicle is required, or for fine patterning, this can be an issue. Accordingly in some embodiments the volume of the culture is kept constant during exposure to the light. Another solution to this issue is to ensure that the pellicle is constantly exposed to light that is in focus.

The skilled person will appreciate that one way of exposing certain areas of a pellicle to light and not others is through the use of a stencil, or mask. A mask comprises regions that are transparent or partially transparent and regions that are fully or partially opaque. It is considered that the amount of light to which the region of pellicle is exposed will influence the degree of pigmentation or melanation in that area. For example, a high intensity of light will cause more of the split-polymerase or split-transcription factor to dimerise and so lead to increased transcription and production of the gene encoded by the third nucleic acid, for example increased production of tyrosinase.

Accordingly in one embodiment the region or regions of the cellulose pellicle that are not to be exposed to light are protected using a mask.

To maintain a sharp edge between exposed and non-exposed pellicle, in some embodiments the mask is placed as close as possible to the surface of the pellicle, and in some embodiments the mask contacts the surface of the pellicle.

In some embodiments the mask is entirely opaque.

In some embodiments the mask comprises opaque regions and transparent regions. For example, in some embodiments the mask comprises at least some regions that are semi-transparent so as to allow a reduced intensity of light to reach the pellicle in at least some areas.

As described above, it is considered that the degree of pigmentation or melanation in the cellulose can be controlled by modifying the expression of the gene encoded by the third nucleic acid, for example the tyrosinase. The expression of tyrosinase can be modulated by varying the intensity or duration of light that the pellicle is exposed to. In embodiments where the first promoter and/or second promoter are inducible promoters, then the expression level of tyrosinase can also be modulated by varying the concentration of inducing agent that the system is exposed to.

Accordingly in some embodiments the strength of expression of the third polypeptide that:
i) is involved in the biosynthesis of a pigment visible to the naked eye or in the biosynthesis of a molecule that emits light, for example a gene that encodes tyrosinase; or
ii) encodes a protein that emits light or is a pigment;
   is modulated by varying:
a) the intensity of light that the pellicle or culture is exposed to; and/or
b) the duration of exposure to light.

In some embodiments where:
i) the first nucleic acid comprises a first promoter operably linked so as to drive expression of the first polypeptide; and
ii) the second nucleic acid comprises a second promoter operably linked so as to drive expression of the second polypeptide,
   and wherein when the first and second promoter are inducible promoters, then the strength of expression of the third polypeptide that:
i) is involved in the biosynthesis of a pigment visible to the naked eye or in the biosynthesis of a molecule that emits light; or
ii) encodes a protein that emits light or is a pigment;
   is modulated by varying:
a) the intensity of light that the pellicle or culture is exposed to;
b) the duration of exposure to light; and/or
c) the concentration of inducing agent that the pellicle or culture is exposed to;
   for example where the first promoter and second promoter are arabinose inducible promoters, the cell is engineered to also express AraC and the inducing agent is arabinose.

In particular embodiments, as already described elsewhere herein, the third polypeptide that:
(a) is involved in the biosynthesis of a pigment visible to the naked eye or in the biosynthesis of a molecule that emits light; or
b) encodes a protein that emits light or is a pigment;
   is an enzyme necessary for the formation of melanin, for example wherein the melanin is selected from the group comprising eumelanin, pheomelanin, neuromelanin and allomelanln. In some of these embodiments the third polypeptide is tyrosinase, for example:
Tyr1 from Bacillus megaterium [SEQ ID NO: 13];
mel from Streptomyces antibiotics [SEQ ID NO: 14]; or
mel from Rhizobium etli [SEQ ID NO: 15];
   or is a tyrosinase comprises or consists of a sequence with at least 80%, 82%, 84%, 86%, 88%, 90%, 92%, 94%, 96%, 98%, 99% or 100% sequence identity to any of the above sequences.

The invention also provides a method for spatially restricted gene expression in bacterial cellulose. For example, in some embodiments the method for spatially restricted gene expression in bacterial cellulose comprises:
(a) providing a culture of the cells of the invention, i.e., cells that comprise the optogenetic expression system according to the invention;
(b) maintaining the culture of (a) under conditions that allow the production of a cellulose pellicle by the cells; and
(c) exposing a defined region or regions of the cellulose pellicle to light of the dimerization wavelength so as to allow dimerization of the first and second light-inducible dimerization domain and formation of the functional heterologous polymerase and transcription of the third nucleic acid sequence that encodes a target protein or RNA to be expressed.

As described for other methods of the invention, in some embodiments the cellulose pellicle in (b) is allowed to develop to the final desired area and/or thickness prior to exposing the defined region or regions to light in step (c). In some embodiments once the pellicle in (b) has developed to the final desired area and/or thickness it is harvested prior to the defined region or regions to light in step (c).

In the same or other embodiments, the spatially defined regions of the cellulose pellicle are exposed to the light during step (b).

Preferences for this method are as described elsewhere herein in relation into other methods. For example, in some embodiments:
i) the volume of the culture is kept constant during exposure to the light;
ii) the region or regions of the cellulose pellicle that are not to be exposed to light are protected using a mask;
iii) the mask is placed as close as possible to the surface of the pellicle, for example wherein the mask contacts the surface of the pellicle;
iv) the mask is entirely opaque;
v) the mask comprises at least some region or regions that are semi-transparent so as to allow a reduced intensity of light to reach the pellicle in at least some areas;
vi) the strength of expression from the third nucleic acid sequence that encodes a target protein or RNA to be expressed is modulated by varying:
   a) the intensity of light that the pellicle or culture is exposed to; and/or
   b) the duration of exposure to light.
vii) where:
   i) the first nucleic acid comprises a first promoter operably linked so as to drive expression of the first polypeptide; and
   ii) the second nucleic acid comprises a second promoter operably linked so as to drive expression of the second polypeptide,
   and wherein the first and second promoter are inducible promoters, then the strength of expression from the third nucleic acid sequence that encodes a target protein or RNA to be expressed is modulated by varying:
      a) the intensity of light that the pellicle or culture is exposed to;
      b) the duration of exposure to light; and/or
      c) the concentration of inducing agent that the pellicle or culture is exposed to;
      optionally where the first promoter and second promoter are arabinose inducible promoters, the inducing agent is arabinose;
   viii) the third nucleic acid sequence encodes an enzyme necessary for the formation of melanin, optionally wherein the melanin is selected from the group comprising eumelanin, pheomelanin, neuromelanin and allomelanin;
   ix) wherein the third nucleic acid encodes:
      Tyr1 from Bacillus megaterium [SEQ ID NO: 13];
      mel from Streptomyces antibiotics [SEQ ID NO: 14]; or
      mel from Rhizobium etli [SEQ ID NO: 15];
      or is a tyrosinase comprises or consists of a sequence with at least 80%, 82%, 84%, 86%, 88%, 90%, 92%, 94%, 96%, 98%, 99% or 100% sequence identity to any of the above sequences.

The invention also provides a method for producing a bacterial cellulose pellicle that can be spatially pigmented upon exposure to light, wherein the method comprises:
(a) providing a culture of the cells of the invention, i.e., cells that comprise the optogenetic expression system of the invention;
(b) maintaining the culture of (a) under conditions that allow the production of a cellulose pellicle by the microorganism until a pellicle of the appropriate area and/or thickness has been obtained; and
(c) harvesting the pellicle; and
wherein the pellicle has not been exposed to light of the dimerization wavelength.

Preferences for this method are as described elsewhere herein in relation to other methods.

The invention also provides a method for spatially pigmenting bacterial cellulose wherein the method comprises:
a) providing a bacterial cellulose pellicle that has been produced by a culture of cells of the invention (i.e., cells that comprise the optogenetic expression system according to the invention); and
b) exposing spatially restricted areas of the pellicle to light of the dimerization wavelength.

Preferences for this method are as described elsewhere herein, for example the preferences for the exposure to light of the dimerization wavelength include exposing to light of the dimerization wavelength for:
at least 1 hour, 2, 3, 4, 5, 6, 7, 8, 9, 11, 12, 18, 24, 36, 48, 60, 72, 84, 96 hours, 5 days, 6 days, 7 days, 2 weeks, 1 month or more; and/or
less than 1 month, less than 2 weeks, 7 days, 6 days, 5 days, 96 hours, 84, 72, 60, 48, 36, 24, 18, 12, 11, 9, 8, 7, 6, 5, 4, 3, 2, 1 hours.

As described above, a pellicle produced by any of the methods of the invention may be sterilised, for example may be sterilised by:
i) autoclaving;
ii) heating; and/or
iii) desiccation, optionally with 70% ethanol.

The invention also provides a bacterial pellicle that has been produced according to any of the methods described herein. In some embodiments, the bacterial pellicle may comprise any cell as described herein, optionally any bacterial cell as described herein.

The invention also provides a spatially pigmented bacterial pellicle as produced according to any of the methods described herein, for example where the pigment is melanin. In some embodiments, the spatially pigmented bacterial pellicle may comprise any cell as described herein, optionally any bacterial cell as described herein.

The invention also provides a pigmented bacterial pellicle as produced according to any method described herein, for example wherein the pigment is melanin. In some embodiments, the pigmented bacterial pellicle may comprise any cell as described herein, optionally any bacterial cell as described herein.

The invention also provides a bacterial pellicle suitable for light-induced spatially restricted pigmentation wherein the bacterial pellicle has been produced according to a method of the invention and wherein the pellicle has not been exposed to light of the dimerization wavelength. In some embodiments, the bacterial pellicle suitable for light-induced spatially restricted pigmentation may comprise any cell as described herein, optionally any bacterial cell as described herein.

As described in the examples, bacterial cellulose can be used to create textiles that can be used in any number of applications, for example clothing, building materials, medical materials. Accordingly, the invention provides a textile comprising a bacterial pellicle that has been produced according to any method of the invention. In some embodiments, the textile comprising a bacterial pellicle may comprise any cell as described herein, optionally any bacterial cell as described herein.

The invention also provides a bacterial pellicle that has spatially defined regions of pigmentation, for example spatially defined regions of melanin pigmentation. In some embodiments, the bacterial pellicle that has spatially defined regions of pigmentation may comprise any cell as described herein, optionally any bacterial cell as described herein.

The invention also provides an apparatus for exposing spatially defined regions of a bacterial pellicle to light comprising:
i) a light source to illuminate a surface of the bacterial pellicle;
ii) a light diffuser
iii) a mask, optionally a transparency; and
iv) a lens;
   wherein the distance between the light source and the surface of the bacterial pellicle can be adjusted. In some embodiments the lens autofocuses on the surface of the pellicle. The light source is preferably a low voltage and/or a low wattage light source, for example a low voltage and/or a low wattage LED flood lamp with a wattage less than 100 W, 80 W, 60 W, 50 W, 40 W, 30 W, 20 W, or 10 W or less; and/or 10 W or more, 20 W, 30 W, 40 W, 50 W, 60 W, 60 W, 100 W or more.

In some embodiments, the light diffuser is an optional component of the apparatus for exposing spatially defined regions of a bacterial pellicle.

The invention also provides the use of the apparatus of the invention in the spatially restricted gene expression of a bacterial pellicle produced according to any of the methods of the invention, for example in some embodiments the gene expression results in pigmentation, in which case the invention also provides the use of the apparatus of the invention in the spatially restricted pigmentation, for example melanation, of a bacterial pellicle produced according to any of the methods of the invention.

The invention provides the use of a digital projector in the spatially restricted pigmentation of a bacterial pellicle produced according to any of the preceding claims. It will be clear that a particularly advantageous feature of the present invention is the development solution that is required to produce the melanin in a cellulose pellicle that comprises tyrosinase. Accordingly, the invention also provides a pigment development solution, wherein the solution:
A) comprises metal ions with an oxidation state of 2+; optionally comprises:
   i) a) Cu²⁺, Zn²⁺, Be²⁺, Mg²⁺, Ca²⁺, Cr²⁺, Mn²⁺, Co²⁺ or N12+;
      b) Cu²⁺, Zn²⁺; and/or
      c) Cu²⁺; and/or
   ii) CuSO₄, optionally comprises:
      at least 2 μM CuSO₄, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuSO₄; and/or
      between 2 μM CuSO₄ and 20 μM CuSO₄; and/or
      less than 20 μM, optionally less than 17.5 μM, 15 μM, 12.5 μM, 10 μM, 7.5 μM, 5 μM, 4 μM, 3 μM, or less than 2 μM CuSO₄; and/or
      between 20 μM and 160 μM CuSO₄; and/or
      less than 160 μM, optionally less than 140 μM, 120 μM, 100 μM, 80 μM, 60 μM, 40 μM, or less than 20 μM CuSO₄; and/or
   iii) CuCl₂, optionally comprises:
      at least 2 μM CuCl₂, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuCl₂; and/or
      between 2 μM CuCl₂ and 20 μM CuCl₂; and/or
      less than 20 μM, optionally less than 17.5 μM, 15 μM, 12.5 μM, 10 μM, 7.5 μM, 5 μM, 4 μM, 3 μM, or less than 2 μM CuCl₂; and/or
      between 20 μM and 160 μM CuCl₂; and/or
      less than 160 μM, optionally less than 140 μM, 120 μM, 100 μM, 80 μM, 60 μM, 40 μM, or less than 20 μM CuCl₂
         and
B) is at a pH of:
   between 6 and 8.5, optionally between 6.25 and 8.25, 6.5 and 8, 6.25 and 7.75, 6.5 and 7.5, 6.75 and 7.25; and/or
   at least 6, optionally at least 6.25, 6.5, 6.75, 7, 7.25, 7.4, 7.5, 7.75, 8, 8.25 or at least 8.5; and/or 7.4;
      and
C) comprises L-tyrosine and/or L-cysteine and/or L-cystine.

In some embodiments the development solution comprises:
i) at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L tyrosine; and/or
less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine;
   and/or
ii) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
between 10 g/L and 300 g/L cysteine; and/or
less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
   and/or
iii) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L L-cystine; and/or
less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine.

In some preferred embodiments the development solution comprises:
a) PBS at pH 7.4;
b) 10 μM CuSO₄, or 20 μM CUSO₄; and
c) 0.5 g/L L-tyrosine or 1 g/L L-tyrosine, and/or 1 g/L L-cysteine and/or 0.4 g/L L-cystine.

The skilled person will recognise that the present invention lends itself to being provided as a kit of parts. For example, the invention provides a kit comprising:
a) a bacterial pellicle of the invention or a bacterial pellicle produced according to any method of the invention; and
b) a development solution according to the invention.

The invention also provides a kit comprising:
a) a bacterial pellicle of the invention or a bacterial pellicle produced according to any method of the invention that comprises an optogenetic expression system according to the invention;
b) a development solution of the invention.

In some embodiments any of the kits also comprises:
c) a light source.

In some embodiments the light source is the apparatus of the invention. In some embodiments the light source provides light of the dimerization wavelength.

The invention also provides a light-responsive bacterial cellulose material. In some embodiments the light-responsive bacterial cellulose material has been produced according to any of the methods described herein. In some embodiments, the light-responsive bacterial cellulose material may comprise any cell as described herein, optionally any bacterial cell as described herein.

The inventors also found that the melanated bacterial cellulose had an increased wettability as compared to the non-melanated bacterial cellulose. Accordingly, the invention provides methods of producing bacterial cellulose with increased wettability, wherein the method comprises producing melanated bacterial cellulose according to any of the methods described herein. The invention also provides bacterial cellulose with spatially restricted regions of increased wettability. In some embodiments, the bacterial cellulose with increased wettability may comprise any cell as described herein, optionally any bacterial cell as described herein.

The invention also provides any product, method, or kit as described substantially herein.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the invention provides:
1) A bacterial pellicle that comprises tyrosinase and that comprises the optogenetic expressions system of the invention, and that has not been exposed to light of the dimerization wavelength, wherein the tyrosinase is Tyr1 and the pellicle was produced by K. rhaeticus;
2) A method for producing melanated bacterial cellulose wherein the method comprises exposing a cellulose pellicle that comprises tyrosinase to a development solution, wherein the development solution:
   comprises metal ions with an oxidation state of 2+;
   is at a pH of between 6 and 8.5; and
   comprises L-tyrosine and/or L-cysteine and/or L-cystine;
wherein the development solution comprises 10 g/L cysteine at pH 6 and also comprises 5 uM CuCl₂;
3) A cell that is a Komagaelbacter rhaeticus that comprises:
an optogenetic expression system for use in bacteria of the genus Komagataeibacter, comprising:
(a) A first nucleic acid comprising a first nucleotide sequence that encodes a first polypeptide, wherein the first polypeptide comprises:
   i) a first domain that comprises a first portion of a heterologous split-polymerase; and
   ii) a second domain that comprises a first light-inducible dimerization domain;
(b) A second nucleic acid comprising a second nucleotide sequence that encodes a second polypeptide, wherein the second polypeptide comprises:
   i) A first domain that comprises a second portion of a heterologous split-polymerase; and
ii) A second domain that comprises a second light-inducible dimerization domain;
and
(c) A third nucleic acid comprising a third nucleic acid sequence that encodes a target RNA to be expressed operably linked to a target promoter;
and wherein the first light-inducible dimerization domain and the second light-Inducible dimerization domain are capable of dimerising with one another upon exposure to light of a dimerization wavelength to form a functional heterologous polymerase or a functional transcription factor capable of transcribing or initiating transcription from the target promoter,
and wherein the target promoter is recognised by the functional heterologous polymerase or functional transcription factor so as to drive transcription of the third nucleic acid sequence that encodes a target protein or RNA,
wherein the first and second light-inducible dimerization domains are VVD domains
wherein the target RNA to be expressed is a gRNA for use in CRISPR editing
and wherein the first nucleic acid, the second nucleic acid and optionally the third nucleic acid are integrated in to the genome of the cell.

### FIGURE LEGENDS

FIG. 1—A phylogenetic tree of the Komagataeibacter genus. The tree was calculated from 260 unique homologous genes across all 17 species and was run through 500 bootstrap cycles. All nodes but one returned a bootstrap score of 100%. The only node that scored less than 100% was the K. nataicola/K. rhaeticus node (shown in orange) that scored 89%. While species names may have originally been published as Gluconacetobacter all are referred to as Komagataeibacter in the tree.

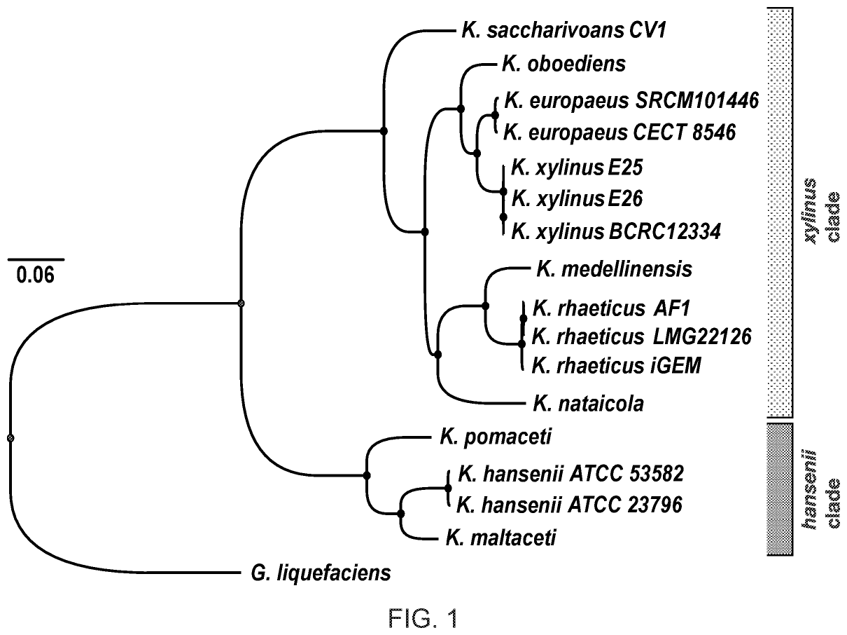

FIG. 2—The bacterial cellulose operon from K. xylinus E25. The arrangement is conserved across all Komagataeibacter; however, many strains will include multiple copies of the cellulose synthase operon. These operons may include the hybridised gene, bcsAB, and may also be missing the flanking genes or bcsC and bcsD.

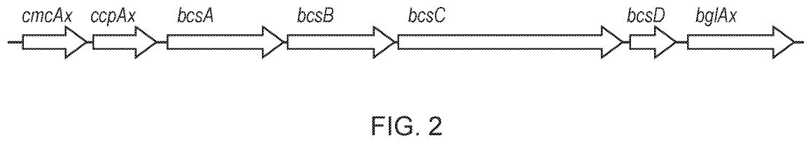

FIG. 3—Active polysaccharide secretion from live K. rhaeticus. A) Time-lapse imagery over 80 minutes showing the secretion of a ribbon like polysaccharide from K. rhaeticus cells. Imagery is a composite of two fields: K. rhaeticus cells are captured using phase contrast brightfield. FB 28 is excited with UV light at 350 nm and emission captured at 470 nm with an exposure time of 10 seconds. B) Diagram reflecting the ribbon form of polysaccharide secretion C) An enlarged view of the FB 28 field at timepoint 80 minutes. The brightness and contrast of the field have been increased to increase the visibility of the details in the ribbons D) A time-lapse over 16 mins showing the secretion of a rope-like polysaccharide morphology. White pointer: The cell pair producing the polysaccharide rope. Grey pointer: the polysaccharide rope. E) Diagram reflecting the rope form of polysaccharide secretion F) An enlarged view of FB 28 field at timepoint 16 minutes. The brightness and contrast have been increased significantly, to make the polysaccharide ropes visible.

FIG. 4—The ptyr1 plasmid for the expression of the Tyr1 protein in K. rhaeticus. Additional plasmid contents not shown are a chloramphenicol resistance gene and pBBR1 origin of replication. The backbone used in construction was d1.2 from the Komagataeibacter Toolkit.

FIG. 5—Identifying conditions favourable for eumelanin synthesis. A) K. rhaeticus ptyr1 cultures grown with shaking and 2% cellulase to prevent clumping. The pH of each media was set and measured before growth. B) Attempts to grow a pellicle in eumelanin producing conditions. Cultures were grown in quadruplicate; a single illustrative example of each condition is show here. Pellicles were grown covered in 24 well deep well plates. The images are taken from above the plate.

FIG. 6—The production of melanated bacterial cellulose. A) The two-step method for producing melanated bacterial cellulose. The process begins on the left with the harvesting of a K. rhaeticus ptyr1 pellicle, which is then washed in a development bath, during which the pellicle darkens, and eumelanin is produced. B) A Time-lapse of two pellicles in a buffered development bath compared to a media bath with the same melanin formation chemicals. C) Control of pellicle shade through altering growth and development conditions. Pellicles were grown simultaneously in 24 well deep well plates before being harvested and developed individually. The top left pellicle acts as a negative control for eumelanin production as it was not exposed to any tyrosine and copper. D) Melanin robustness, with the exception of the UV and autoclaved, all pellicles were placed in the conditions for 3 hours.

FIG. 7—The impact of eumelanin production on K. rhaeticus A) A spun down tube of cells from a shaking culture of K. rhaeticus ptyr1. B) Microscopy images of melanated K. rhaeticus cells. The first two columns show imagery from light microscopy. Cells were imaged on top of an agarose pad, which kept bacteria within the same plane of focus. A zoomed image of a single cell is show for both melanated and unmelanated cells. The right most stack of images is the result of TEM. Dark cells can be observed in both images, however as this is TEM, this is not a reflection of the cell colour. In both images a large amount of cellulose, which takes the form of fluffy clumps, can be seen despite the use of cellulase in the culturing process. In the top unmelanated cells image, a number of light cell shaped objects can be seen, we believe these to be cellulose casts of cells.

FIG. 8—The effect of melanin production on bacterial cellulose material properties. A) A series of SEM images of unmelanated (top) and melanated (bottom) pellicles. Cross-section were prepared by cutting freeze dried pellicles. B) The sessile drop method was used to measure the contact angle on 7 unmelanated and 8 melanated pellicles. The average contact angle for the unmelanated pellicles and melanated pellicles were 47° and 28° respectively. The top and bottom images show representative waterdrop shapes on unmelanated and unmelanated pellicles. C) Tensile testing was conducted on 7 unmelanated and 5 melanated pellicles. The left and right images show typical end breaks for unmelanated and melanated pellicles respectively. The average tensile strength values were 112 MPa and 126 MPa for unmelanated and melanated pellicles respectively. For the Young's modulus, the values were 14.7 GPa and 16.2 GPa for unmelanated and melanated pellicles respectively.

FIG. 9—The T7-Opto system adapted for K. rhaeticus. A) Genetic construct for the T7-Opto system in K. rhaeticus. The plasmid construct uses a pBBR1 plasmid origin and has chloramphenicol resistance. The araC gene is under low constitutive expression. The original RBS and terminators in the T7-Opto system, and the split T7 RNA polymerase and mCherry genes were conserved in this adapted construct B) Two cultures of K. rhaeticus pT7-Opto, the left grown in darkness and the right grown in a blue light illuminated incubator. The cultures have been pelleted to increase the visibility of the mCherry accumulation. C) The fold changes between cultures grown in darkness and under blue light cultured on HS media containing 2% (w/v) of differing carbon sources.

FIG. 10—Characterisation of the K. rhaeticus pT7-Opto. A) A simple method for plate based optogenetics experiments. The tablet computer was set specifically to only display blue light by displaying full screen image set to pure blue (RGB, standing for, red, green, blue, is a colour standard for representing colour displayed on screens.). The shading on the transparency is presented as K %, this measure is derived from the CMYK (cyan, magenta, yellow, black) colour standard for printing. A K of 0% corresponds to no black ink being printed on to the transparency, whilst 100% K is the maximum density of black the printer can lay down. How much light is let though therefore varies from printer to printer, and 100% may not be fully opaque. B) The dose response curves of K. rhaeticus pT7-Opto over four separate concentrations of arabinose, displayed in % (w/v). The tablet was adhered to the floor of a shaking incubator and the cells were incubated with 2% cellulase and shaking for 24 hours. The mCherry fluorescence accumulation was measured using flow cytometry.

FIG. 11—The Enlarger v1, a proof-of-concept method for creating patterned gene expression in growing bacterial cellulose. A) A rendering of the assembled enlarger. The full assembly measures 27 cm in height, and when in use, can be fitted into a small desktop incubator. B) An exploded view of each component in the Enlarger V1. The LED lamp is run on mains electricity and remains plugged in during use. The projection lens used has a 50 mm focal length and during use, the aperture is set to f/2.8. The lens is focused before pellicle growth to the surface height of 100 ml of liquid in the culture container. The culture container is show here as clear to better understand the location of the pellicle, however during use the container is covered to block outside light from effecting the projection.

FIG. 12—A patterned K. rhaeticus pT7-Opto pellicle from the Enlarger V1. A) A high-resolution scan of the patterned pellicle. The face shown is the top of the pellicle. In the image, white is used to represent the detection of no red fluorescence whilst a stronger intensity of red reflects increased red fluorescence. B) The pixel intensity distributions of exposed and unexposed regions of the pellicle. The fluorescent scan used a 16-bit image depth, representing 65536 levels of intensity for each pixel. The average pixel intensity of the unexposed region was 5516 (grey curve), whilst the average intensity of the exposed region was 10413 (red curve). Due to the difference in the number of pixels in the exposed and unexposed areas, the distributions were normalised to each other to improve the comparison.

FIG. 13—Enlarger v2. An iteration on the Enlarger v1. The assembly is 620 mm high and due to this size does not fit into most incubators. Therefore, the Enlarger is enclosed in a cardboard box during use, which traps the heat produced by the lamp and maintains an internal temperature of ˜30° C. The printed transparency measures 27×26 mm and is composed of 4 copies of printed acetate stacked a top each other. The lens is 80-200 mm and the aperture used during is printing is f/4. The distance between the transparency and the top of the lens is 40 mm and the distance between the bottom of the lens and the bottom of the culture dish is 410 mm. The blackout hood is shown as transparent here for clarity, but in reality, is composed of a thick opaque white fabric. It ensures that the only blue light that reaches the growing pellicle is projected from the lens. The pellicle culture dish is also covered during growth with a glass lid to prevent contamination.

FIG. 14—Test cards for the standardisation of visual equipment. A) A pair of test cards used by the BBC (top) and EBU (bottom). The cards carry multiple tests for the testing and calibration of visual equipment. B) A test card for patterned bacterial cellulose. The card carries tests for visual acuity based on background, text rendering, tests for determining the minimum discernible feature size and placing the dynamic range of the test card onto the dose response of the T7-Opto system. The test card is printed onto acetate and placed into the Enlarger V2 for printing onto a K. rhaeticus pT7-Opto pellicle.

FIG. 15—Test card printed onto a bacterial cellulose pellicle. A) A 16-bit fluorescence scan of the top surface of a patterned K. rhaeticus pT7-Opto pellicle. A white to red look up table is used to represent fluorescence intensity. The projected test card image was slightly too large, meaning the left most exposure test has been partially cut off. B) An exposure test for the patterned K. rhaeticus pT7-Opto pellicle. In the test card, a gradient from 100% to 0% density black ink, which spans the full range of possible printed shades, is used to determine the exposure of the pellicle. The test assumes that the fluorescence response from a pellicle exposed to light across its whole dynamic range would be sigmoidal. If the exposure of the pellicle is centred, the test should show a sigmoidal response, centred half-way along the gradient strip. If the pellicle is overexposed, this sigmoidal response would be shifted left and if overexposed, shifted right. The red curve in the graph represented the average pixel intensity for each horizontal line of pixels exposed to gradient strip, and the two grey lines represent the standard deviation. C) The Imperial College London logo used to determine how well text can be read from a printed pellicle. D) Determining whether a dark or light background produces better definition images E) A range of discernible shades—the point at which shades can no longer be separated from their background is indicated by the triangles. F) Determining the smallest possible printed point—a representation of the highest possible printable resolution. A series of smaller and smaller squares, on both dark and light backgrounds, is used to determine at which point a mark could no longer be deciphered, by eye, in the final printed pellicle. The size of each point in the final printed pellicle is displayed above and below each square.

FIG. 16—Using the T7-Opto system to pattern eumelanin production. A) The layout of the PT7-Opto_tyr1 for producing tyrosinase in response to blue light. With the exception of replacing the mCherry gene for the tyr1 gene, the system is unchanged from the original PT7-Opto plasmid. B) The image projected onto the growing K. rhaeticus PT7-Opto_tyr1 pellicle. Based upon the London Underground roundel (above). The image (below) is created through a mask that sits above the lens in the Enlarger v1. C) The harvested K. rhaeticus PT7-Opto_tyr1 pellicle after light exposure, placed onto a lightbox. The region included in the orange block is replicated to the right, with a dashed outline of the region exposed to blue light. D) From left to right, a time-lapse of the K. rhaeticus PT7-Opto_tyr1 pellicle melanin development process over 4 hours. After the final image, the pellicle was removed from the development bath to prevent over development. A dashed overlay of the region exposed to blue light is displayed on the first image.

FIG. 17—The production of a melanated bacterial cellulose shoe upper. Top image shows the pellicle fully grown within the shoe upper loom. The patches of lower density were the result of mould contamination. The three images in the row below show the pellicle as it undergoes the melanin development process. Over time the surface of the buffer develops a reflective sheen that makes viewing the pellicle development difficult. Image directly above shows the final melanated shoe upper after trimming away the excess cellulose. Here the intensity of blackness can be easily observed. Directly left, shows the process of lasting the melanated shoe upper around a last to form the final shoe shape.

FIG. 18—Bacterial cellulose is produced in copious amounts by Komagataeibacter species. Left panel: a hydrated pellicle (also referred to as never-dried pellicle) grown on HS media. Without the tannins found in tea, the pellicle is normally pale or white in colour. Right panel: A dried cellulose pellicle. Unless dried while pressed flat, the pellicle will take on a wrinkled look. The dried cellulose is relatively stiff but bends without braking.

FIG. 19—Investigating the blue-light responses to variants of a separated T7-opto system in K. rhaeticus A) An adapted T7-opto system, split into chromosomal and plasmid components. Five variations of the Opto-T7RNAP blue light sensor from Baumschlager et al. and araC regulator gene were integrated into K. rhaeticus. The target genes, under the T7 promoter, were placed onto a separate spectinomycin resistant pBBR1 plasmid, which is transformed into K. rhaeticus carrying Opto-T7RNAP blue light sensors. B) Blue light response assays for chromosomal and plasmid optogenetic systems. Data in grey show responses to blue-light exposed cells, whilst data in black shows responses for cells grown in darkness. Error bars correspond to standard deviation of 3 biological replicates. Data for melanin production represent the initial reaction rate, i.e., the initial rate at which light at 405 nm is absorbed by cells in melanin producing conditions.

FIG. 20—Spatial control of melanin accumulation in a pellicle. A) Render of the projector rig used in pellicle optogenetic experiments. The N-mag and P-mag domains are sensitive to light of ˜450 nm, which fits within the blue output of the LED projector, we do however also test other colours that contain blue light, namely, cyan (blue and green light) and white (blue, green and red light). B) Timelapse image that is projected on to the growing bacterial cellulose pellicle. The grid of blocks act as a timer. At the beginning of the timelapse, hour 0, only one block of each row is displayed. Every 8 hours another is added, after hour 72, the 10^{th} and final block is added to make the full row, as displayed in the figure. C) A photograph and densitometric scan of the harvested and developed pellicle. The photographic image is taken with a white background, so that less dense regions appear lighter, and more melanated regions darker. The overall pellicle size is ˜300 mm×170 mm. The orange arrows in the densitometric scan point to the clearest blocks of increased melanin from the timer.

FIG. 21—Melanin production assay in a range of pH conditions. On the left are shown the melanin accumulation and initial reaction rates of K. rhaeticus tyr1, and on the right are the initial reaction rates of K. rhaeticus WT. Absorbance reads were taken at OD₄₀₅ every 10 minutes over 12 hours. Points and bars are coloured according to the pH value of the sample. Initial reaction rates were determined using reads from the first 140 minutes of each reaction. N=6 for each reaction condition, error bars represent the standard deviation of all replicates.

FIG. 22—Melanin production assay in differing concentrations of PBS buffer. On the left are shown the melanin accumulation and initial reaction rates of K. rhaeticus tyr1, and on the right are the initial reaction rates of K. rhaeticus WT. Absorbance reads were taken at OD₄₀₅ every 10 minutes over 12 hours. Points and bars are coloured according to PBS the concentration of the sample. Initial reaction rates were determined using reads from the first 140 minutes of each reaction. N=6 for each reaction condition, error bars represent the standard deviation of all replicates.

FIG. 23—Melanin production assay on a selection of metal ions in oxidation state 2+. On the left are shown the initial reaction rates of K. rhaeticus tyr1, and on the right are the initial reaction rates of K. rhaeticus WT. Initial reaction rates were determined using reads from the first 140 minutes of each reaction. N=6 for each reaction condition, error bars represent the standard deviation of all replicates.

FIG. 24—Melanin production assay with different concentrations of Copper (II) sulphate. On the left are shown the initial reaction rates of K. rhaeticus tyr1, and on the right are the initial reaction rates of K. rhaeticus WT. Initial reaction rates were determined using reads from after the first 40 minutes and until 200 minutes for each reaction. N=6 for each reaction condition, error bars represent the standard deviation of all replicates.

FIG. 25—Melanin production assay with differing tyrosine concentration. On the left are shown the initial reaction rates of K. rhaeticus tyr1, and on the right are the initial reaction rates of K. rhaeticus WT. Initial reaction rates were determined using reads from after the first 40 minutes and until 200 minutes for each reaction. N=6 for each reaction condition, error bars represent the standard deviation of all replicates.

FIG. 26—Melanin production assay in different temperatures. Shown are the initial reaction rates of K. rhaeticus tyr1 cells in a melanin development buffer in temperatures between 25° C. and 50° C. Initial reaction rates were determined using reads from after the first 40 minutes and until 200 minutes for each reaction. N=2 for each reaction condition.

Sequences referred to herein:

>SEQ_ID_NO:_1_first_portion_of_the_heterologous_T7_split-polymerase_protein_sequence
MNTINIAKNDFSDIELAAIPFNTLADHYGERLAREQLALERESYEMGEARFRKMFERQLKAGEVADNAAAKPLITTLLPKMIARINDWFE
EVKAKRGKRPTAFQFLQEIKPEAVAYITIKTTLACLTSADNTTVQAVASAIGRAIEDEARFGRIRDLEAKHFKKNVEEQLNKRVGHVYKK
AFMQVVEADMLSKGLLGGEAWSSWHKEDSIHVGVRCIEMLIESTGMVSLHRQNAGVVGQDSETIELAPEYAEAIATRAGALAGISPMFQP
CVVPPKPWTGITGGGYWANGRRPLALVRTHSKKALMRYEDVYMPEVYKAINIAQNTAWKINKKVLAVANVITKWKHCPVEDIPAIEREEL
PMKPEDIDMNPEALTAWKRAAAAVYRKDKARKSRRISLEFMLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENTWWAEQDSPFCFLAFCFEYAGVQHHGLSYNCSLPLAFDGSC
SGIQHFSAMLRDEVGGRAVNLLP
>SEQ_ID_NO:_2_second_portion_of_the_heterologous_T7_split-polymerase_protein_sequence
SETVQDIYGIVAKKVNEILQADAINGTDNEVVTVTDENTGEISEKVKLGTKALAGQWLAYGVTRSVIKSSVMTLAYGSKEFGFRQQVLED
TIQPAIDSGKGLMFTQPNQAAGYMAKLIWESVSVTVVAAVEAMNWLKSAAKLLAAEVKDKKTGEILRKRCAVHWVTPDGFPVWQEYKKPI
QTRLNLMFLGQFRLQPTINTNKDSEIDAHKQESGIAPNFVHSQDGSHLRKTVVWAHEKYGIESFALIHDSFGTIPADAANLFKAVRETMV
DTYESCDVLADFYDQFADQLHESQLDKMPALPAKGNLNLRDILESDFAFA
>SEQ_ID_NO:_3_first_portion_of_the_heterologous_split-polymerase_nucleic_acid_sequence
ATGAACACGATTAACATCGCTAAGAACGACTTCTCTGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAGCACGCTTCCGCAAGATGTTTGAGCGTCAACTTAAA
GCTGGTGAGGTTGCGGATAACGCTGCCGCCAAGCCTCTCATCACTACCCTACTCCCTAAGATGATTGCACGCATCAACGACTGGTTTGAG
GAAGTGAAAGCTAAGCGCGGCAAGCGCCCGACAGCCTTCCAGTTCCTGCAAGAAATCAAGCCGGAAGCCGTAGCGTACATCACCATTAAG
ACCACTCTGGCTTGCCTAACCAGTGCTGACAATACAACCGTTCAGGCTGTAGCAAGCGCAATCGGTCGGGCCATTGAGGACGAGGCTCGC
TTCGGTCGTATCCGTGACCTTGAAGCTAAGCACTTCAAGAAAAACGTTGAGGAACAACTCAACAAGCGCGTAGGGCACGTCTACAAGAAA
GCATTTATGCAAGTTGTCGAGGCTGACATGCTCTCTAAGGGTCTACTCGGTGGCGAGGCGTGGTCTTCGTGGCATAAGGAAGACTCTATT
CATGTAGGAGTACGCTGCATCGAGATGCTCATTGAGTCAACCGGAATGGTTAGCTTACACCGCCAAAATGCTGGCGTAGTAGGTCAAGAC
TCTGAGACTATCGAACTCGCACCTGAATACGCTGAGGCTATCGCAACCCGTGCAGGTGCGCTGGCTGGCATCTCTCCGATGTTCCAACCT
TGCGTAGTTCCTCCTAAGCCGTGGACTGGCATTACTGGTGGTGGCTATTGGGCTAACGGTCGTCGTCCTCTGGCGCTGGTGCGTACTCAC
AGTAAGAAAGCACTGATGCGCTACGAAGACGTTTACATGCCTGAGGTGTACAAAGCGATTAACATTGCGCAAAACACCGCATGGAAAATC
AACAAGAAAGTCCTAGCGGTCGCCAACGTAATCACCAAGTGGAAGCATTGTCCGGTCGAGGACATCCCTGCGATTGAGCGTGAAGAACTC
CCGATGAAACCGGAAGACATCGACATGAATCCTGAGGCTCTCACCGCGTGGAAACGTGCTGCCGCTGCTGTGTACCGCAAGGACAAGGCT
CGCAAGTCTCGCCGTATCAGCCTTGAGTTCATGCTTGAGCAAGCCAATAAGTTTGCTAACCATAAGGCCATCTGGTTCCCTTACAACATG
GACTGGCGCGGTCGTGTTTACGCTGTGTCAATGTTCAACCCGCAAGGTAACGATATGACCAAAGGACTGCTTACGCTGGCGAAAGGTAAA
CCAATCGGTAAGGAAGGTTACTACTGGCTGAAAATCCACGGTGCAAACTGTGCGGGTGTCGATAAGGTTCCGTTCCCTGAGCGCATCAAG
TTCATTGAGGAAAACCACGAGAACATCATGGCTTGCGCTAAGTCTCCACTGGAGAACACTTGGTGGGCTGAGCAAGATTCTCCGTTCTGC
TTCCTTGCGTTCTGCTTTGAGTACGCTGGGGTACAGCACCACGGCCTGAGCTATAACTGCTCCCTTCCGCTGGCGTTTGACGGGTCTTGC
TCTGGCATCCAGCACTTCTCCGCGATGCTCCGAGATGAGGTAGGTGGTCGCGCGGTTAACTTGCTTCCT
>SEQ_ID_NO:_4_second _portion_of_the_heterologous_T7_split-polymerase_nucleic_acid_sequence
AGTGAAACCGTTCAGGACATCTACGGGATTGTTGCTAAGAAAGTCAACGAGATTCTACAAGCAGACGCAATCAATGGGACCGATAACGAA
GTAGTTACCGTGACCGATGAGAACACTGGTGAAATCTCTGAGAAAGTCAAGCTGGGCACTAAGGCACTGGCTGGTCAATGGCTGGCTTAC
GGTGTTACTCGCAGTGTGACTAAGAGTTCAGTCATGACGCTGGCTTACGGGTCCAAAGAGTTCGGCTTCCGTCAACAAGTGCTGGAAGAT
ACCATTCAGCCAGCTATTGATTCCGGCAAGGGTCTGATGTTCACTCAGCCGAATCAGGCTGCTGGATACATGGCTAAGCTGATTTGGGAA
TCTGTGAGCGTGACGGTGGTAGCTGCGGTTGAAGCAATGAACTGGCTTAAGTCTGCTGCTAAGCTGCTGGCTGCTGAGGTCAAAGATAAG
AAGACTGGAGAGATTCTTCGCAAGCGTTGCGCTGTGCATTGGGTAACTCCTGATGGTTTCCCTGTGTGGCAGGAATACAAGAAGCCTATT
CAGACGCGCTTGAACCTGATGTTCCTCGGTCAGTTCCGCTTACAGCCTACCATTAACACCAACAAAGATAGCGAGATTGATGCACACAAA
CAGGAGTCTGGTATCGCTCCTAACTTTGTACACAGCCAAGACGGTAGCCACCTTCGTAAGACTGTAGTGTGGGCACACGAGAAGTACGGA
ATCGAATCTTTTGCACTGATTCACGACTCCTTCGGTACCATTCCGGCTGACGCTGCGAACCTGTTCAAAGCAGTGCGCGAAACTATGGTT
GACACATATGAGTCTTGTGATGTACTGGCTGATTTCTACGACCAGTTCGCTGACCAGTTGCACGAGTCTCAATTGGACAAAATGCCAGCA
CTTCCGGCTAAAGGTAACTTGAACCTCCGTGACATCTTAGAGTCGGACTTCGCGTTCGCG
>SEQ_ID_NO:_5_nMag_dimerization_domain_protein_sequence
HTLYAPGGYDIMGYLDQIGNRPNPQVELGPVDTSCALILCDLKQKDTPIVYASEAFLYMTGYSNAEVLGRNCRFLQSPDGMVKPKSTRKY
VDSNTINTIRKAIDRNAEVQVEVVNFKKNGQRFVNFLTIIPVRDETGEYRYSMGFQCETE
>SEQ_ID_NO:_6 pMag_dimerization_domain_protein_sequence
MHTLYAPGGYDIMGYLRQIRNRPNPQVELGPVDTSCALILCDLKQKDTPVVYASEAFLYMTGYSNAEVLGRNCRFLQSPDGMVKPKSTRK
YVDSNTINTMRKAIDRNAEVQVEVVNFKKNGQRFVNFLTMIPVRDETGEYRYSMGFQCETE
>SEQ_ID_NO:_7_nMag_dimerization_domain_nucleic_acid_sequence
CACACTCTTTACGCCCCTGGAGGATACGACATTATGGGATATTTGGATCAGATTGGGAACCGCCCAAACCCTCAGGTCGAACTGGGGCCT
GTGGACACGTCATGTGCCCTGATCCTGTGCGATCTGAAGCAAAAGGACACTCCGATCGTCTACGCCTCGGAAGCCTTCTTGTATATGACC
GGATACAGCAATGCAGAGGTGCTCGGCAGGAACTGCAGATTCCTGCAGTCCCCCGACGGGATGGTGAAACCAAAGTCGACTCGCAAATAT
GTGGACTCGAACACGATCAACACCATCCGGAAGGCCATCGACCGGAACGCCGAGGTCCAGGTGGAGGTGGTCAACTTTAAGAAGAACGGC
CAGCGGTTCGTGAACTTTCTGACCATCATTCCGGTCCGGGATGAAACCGGAGAGTACAGATACTCCATGGGATTCCAGTGCGAAACCGAA
>SEQ_ID_NO:_8_pMag_dimerization_domain_nucleic_acid_sequence
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGaTCCTGTGCGATCTGAAGCAAAAGGACACTCCGGTGGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAA
>SEQ_ID_NO:_9_First_split-pol_domain_and_nMag_nucleic_acid_sequence
ATGAACACGATTAACATCGCTAAGAACGACTTCTCTGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAGCACGCTTCCGCAAGATGTTTGAGCGTCAACTTAAA
GCTGGTGAGGTTGCGGATAACGCTGCCGCCAAGCCTCTCATCACTACCCTACTCCCTAAGATGATTGCACGCATCAACGACTGGTTTGAG
GAAGTGAAAGCTAAGCGCGGCAAGCGCCCGACAGCCTTCCAGTTCCTGCAAGAAATCAAGCCGGAAGCCGTAGCGTACATCACCATTAAG
ACCACTCTGGCTTGCCTAACCAGTGCTGACAATACAACCGTTCAGGCTGTAGCAAGCGCAATCGGTCGGGCCATTGAGGACGAGGCTCGC
TTCGGTCGTATCCGTGACCTTGAAGCTAAGCACTTCAAGAAAAACGTTGAGGAACAACTCAACAAGCGCGTAGGGCACGTCTACAAGAAA
GCATTTATGCAAGTTGTCGAGGCTGACATGCTCTCTAAGGGTCTACTCGGTGGCGAGGCGTGGTCTTCGTGGCATAAGGAAGACTCTATT
CATGTAGGAGTACGCTGCATCGAGATGCTCATTGAGTCAACCGGAATGGTTAGCTTACACCGCCAAAATGCTGGCGTAGTAGGTCAAGAC
TCTGAGACTATCGAACTCGCACCTGAATACGCTGAGGCTATCGCAACCCGTGCAGGTGCGCTGGCTGGCATCTCTCCGATGTTCCAACCT
TGCGTAGTTCCTCCTAAGCCGTGGACTGGCATTACTGGTGGTGGCTATTGGGCTAACGGTCGTCGTCCTCTGGCGCTGGTGCGTACTCAC
AGTAAGAAAGCACTGATGCGCTACGAAGACGTTTACATGCCTGAGGTGTACAAAGCGATTAACATTGCGCAAAACACCGCATGGAAAATC
AACAAGAAAGTCCTAGCGGTCGCCAACGTAATCACCAAGTGGAAGCATTGTCCGGTCGAGGACATCCCTGCGATTGAGCGTGAAGAACTC
CCGATGAAACCGGAAGACATCGACATGAATCCTGAGGCTCTCACCGCGTGGAAACGTGCTGCCGCTGCTGTGTACCGCAAGGACAAGGCT
CGCAAGTCTCGCCGTATCAGCCTTGAGTTCATGCTTGAGCAAGCCAATAAGTTTGCTAACCATAAGGCCATCTGGTTCCCTTACAACATG
GACTGGCGCGGTCGTGTTTACGCTGTGTCAATGTTCAACCCGCAAGGTAACGATATGACCAAAGGACTGCTTACGCTGGCGAAAGGTAAA
CCAATCGGTAAGGAAGGTTACTACTGGCTGAAAATCCACGGTGCAAACTGTGCGGGTGTCGATAAGGTTCCGTTCCCTGAGCGCATCAAG
TTCATTGAGGAAAACCACGAGAACATCATGGCTTGCGCTAAGTCTCCACTGGAGAACACTTGGTGGGCTGAGCAAGATTCTCCGTTCTGC
TTCCTTGCGTTCTGCTTTGAGTACGCTGGGGTACAGCACCACGGCCTGAGCTATAACTGCTCCCTTCCGCTGGCGTTTGACGGGTCTTGC
TCTGGCATCCAGCACTTCTCCGCGATGCTCCGAGATGAGGTAGGTGGTCGCGCGGTTAACTTGCTTCCTggcggtTCTggaggtCACACT
CTTTACGCCCCTGGAGGATACGACATTATGGGATATTTGGATCAGATTGGGAACCGCCCAAACCCTCAGGTCGAACTGGGGCCTGTGGAC
ACGTCATGTGCCCTGATCCTGTGCGATCTGAAGCAAAAGGACACTCCGATCGTCTACGCCTCGGAAGCCTTCTTGTATATGACCGGATAC
AGCAATGCAGAGGTGCTCGGCAGGAACTGCAGATTCCTGCAGTCCCCCGACGGGATGGTGAAACCAAAGTCGACTCGCAAATATGTGGAC
TCGAACACGATCAACACCATCCGGAAGGCCATCGACCGGAACGCCGAGGTCCAGGTGGAGGTGGTCAACTTTAAGAAGAACGGCCAGCGG
TTCGTGAACTTTCTGACCATCATTCCGGTCCGGGATGAAACCGGAGAGTACAGATACTCCATGGGATTCCAGTGCGAAACCGAATAA
>SEQ_ID_NO:_10_First_split-pol_domain_and_pMag_nucleic_acid_sequence
ATGAACACGATTAACATCGCTAAGAACGACTTCTCTGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAGCACGCTTCCGCAAGATGTTTGAGCGTCAACTTAAA
GCTGGTGAGGTTGCGGATAACGCTGCCGCCAAGCCTCTCATCACTACCCTACTCCCTAAGATGATTGCACGCATCAACGACTGGTTTGAG
GAAGTGAAAGCTAAGCGCGGCAAGCGCCCGACAGCCTTCCAGTTCCTGCAAGAAATCAAGCCGGAAGCCGTAGCGTACATCACCATTAAG
ACCACTCTGGCTTGCCTAACCAGTGCTGACAATACAACCGTTCAGGCTGTAGCAAGCGCAATCGGTCGGGCCATTGAGGACGAGGCTCGC
TTCGGTCGTATCCGTGACCTTGAAGCTAAGCACTTCAAGAAAAACGTTGAGGAACAACTCAACAAGCGCGTAGGGCACGTCTACAAGAAA
GCATTTATGCAAGTTGTCGAGGCTGACATGCTCTCTAAGGGTCTACTCGGTGGCGAGGCGTGGTCTTCGTGGCATAAGGAAGACTCTATT
CATGTAGGAGTACGCTGCATCGAGATGCTCATTGAGTCAACCGGAATGGTTAGCTTACACCGCCAAAATGCTGGCGTAGTAGGTCAAGAC
TCTGAGACTATCGAACTCGCACCTGAATACGCTGAGGCTATCGCAACCCGTGCAGGTGCGCTGGCTGGCATCTCTCCGATGTTCCAACCT
TGCGTAGTTCCTCCTAAGCCGTGGACTGGCATTACTGGTGGTGGCTATTGGGCTAACGGTCGTCGTCCTCTGGCGCTGGTGCGTACTCAC
AGTAAGAAAGCACTGATGCGCTACGAAGACGTTTACATGCCTGAGGTGTACAAAGCGATTAACATTGCGCAAAACACCGCATGGAAAATC
AACAAGAAAGTCCTAGCGGTCGCCAACGTAATCACCAAGTGGAAGCATTGTCCGGTCGAGGACATCCCTGCGATTGAGCGTGAAGAACTC
CCGATGAAACCGGAAGACATCGACATGAATCCTGAGGCTCTCACCGCGTGGAAACGTGCTGCCGCTGCTGTGTACCGCAAGGACAAGGCT
CGCAAGTCTCGCCGTATCAGCCTTGAGTTCATGCTTGAGCAAGCCAATAAGTTTGCTAACCATAAGGCCATCTGGTTCCCTTACAACATG
GACTGGCGCGGTCGTGTTTACGCTGTGTCAATGTTCAACCCGCAAGGTAACGATATGACCAAAGGACTGCTTACGCTGGCGAAAGGTAAA
CCAATCGGTAAGGAAGGTTACTACTGGCTGAAAATCCACGGTGCAAACTGTGCGGGTGTCGATAAGGTTCCGTTCCCTGAGCGCATCAAG
TTCATTGAGGAAAACCACGAGAACATCATGGCTTGCGCTAAGTCTCCACTGGAGAACACTTGGTGGGCTGAGCAAGATTCTCCGTTCTGC
TTCCTTGCGTTCTGCTTTGAGTACGCTGGGGTACAGCACCACGGCCTGAGCTATAACTGCTCCCTTCCGCTGGCGTTTGACGGGTCTTGC
TCTGGCATCCAGCACTTCTCCGCGATGCTCCGAGATGAGGTAGGTGGTCGCGCGGTTAACTTGCTTCCTggcggtTCTggaggtCACACT
CTTTACGCCCCTGGAGGATACGACATTATGGGATATTTGCGGCAGATTAGGAACCGCCCAAACCCTCAGGTCGAACTGGGGCCTGTGGAC
ACGTCATGTGCCCTGaTCCTGTGCGATCTGAAGCAAAAGGACACTCCGGTGGTCTACGCCTCGGAAGCCTTCTTGTATATGACCGGATAC
AGCAATGCAGAGGTGCTCGGCAGGAACTGCAGATTCCTGCAGTCCCCCGACGGGATGGTGAAACCAAAGTCGACTCGCAAATATGTGGAC
TCGAACACGATCAACACCATGCGGAAGGCCATCGACCGGAACGCCGAGGTCCAGGTGGAGGTGGTCAACTTTAAGAAGAACGGCCAGCGG
TTCGTGAACTTTCTGACCATGATTCCGGTCCGGGATGAAACCGGAGAGTACAGATACTCCATGGGATTCCAGTGCGAAACCGAATAA
>SEQ_ID_NO:_11_Second_split-pol_domain_and_nMag_nucleic_acid_sequence
ATGCACACTCTTTACGCCCCTGGAGGATACGACATTATGGGATATTTGGATCAGATTGGGAACCGCCCAAACCCTCAGGTCGAACTGGGG
CCTGTGGACACGTCATGTGCCCTGATCCTGTGCGATCTGAAGCAAAAGGACACTCCGATCGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATCCGGAAGGCCATCGACCGGAACGCCGAGGTCCAGGTGGAGGTGGTCAACTTTAAGAAGAAC
GGCCAGCGGTTCGTGAACTTTCTGACCATCATTCCGGTCCGGGATGAAACCGGAGAGTACAGATACTCCATGGGATTCCAGTGCGAAACC
GAAggcggtTCTggaggtAGTGAAACCGTTCAGGACATCTACGGGATTGTTGCTAAGAAAGTCAACGAGATTCTACAAGCAGACGCAATC
AATGGGACCGATAACGAAGTAGTTACCGTGACCGATGAGAACACTGGTGAAATCTCTGAGAAAGTCAAGCTGGGCACTAAGGCACTGGCT
GGTCAATGGCTGGCTTACGGTGTTACTCGCAGTGTGACTAAGAGTTCAGTCATGACGCTGGCTTACGGGTCCAAAGAGTTCGGCTTCCGT
CAACAAGTGCTGGAAGATACCATTCAGCCAGCTATTGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAATGAACTGGCTTAAGTCTGCTGCTAAGCTGCTGGCT
GCTGAGGTCAAAGATAAGAAGACTGGAGAGATTCTTCGCAAGCGTTGCGCTGTGCATTGGGTAACTCCTGATGGTTTCCCTGTGTGGCAG
GAATACAAGAAGCCTATTCAGACGCGCTTGAACCTGATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCCAAGACGGTAGCCACCTTCGTAAGACTGTAGTGTGG
GCACACGAGAAGTACGGAATCGAATCTTTTGCACTGATTCACGACTCCTTCGGTACCATTCCGGCTGACGCTGCGAACCTGTTCAAAGCA
GTGCGCGAAACTATGGTTGACACATATGAGTCTTGTGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCTTAGAGTCGGACTTCGCGTTCGCGTAA
>SEQ_ID_NO:_12_Second_split-pol_domain_and_pMag_nucleic_acid_sequence
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGaTCCTGTGCGATCTGAAGCAAAAGGACACTCCGGTGGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAAggcggtTCTggaggtAGTGAAACCGTTCAGGACATCTACGGGATTGTTGCTAAGAAAGTCAACGAGATTCTACAAGCAGACGCAATC
AATGGGACCGATAACGAAGTAGTTACCGTGACCGATGAGAACACTGGTGAAATCTCTGAGAAAGTCAAGCTGGGCACTAAGGCACTGGCT
GGTCAATGGCTGGCTTACGGTGTTACTCGCAGTGTGACTAAGAGTTCAGTCATGACGCTGGCTTACGGGTCCAAAGAGTTCGGCTTCCGT
CAACAAGTGCTGGAAGATACCATTCAGCCAGCTATTGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAATGAACTGGCTTAAGTCTGCTGCTAAGCTGCTGGCT
GCTGAGGTCAAAGATAAGAAGACTGGAGAGATTCTTCGCAAGCGTTGCGCTGTGCATTGGGTAACTCCTGATGGTTTCCCTGTGTGGCAG
GAATACAAGAAGCCTATTCAGACGCGCTTGAACCTGATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCCAAGACGGTAGCCACCTTCGTAAGACTGTAGTGTGG
GCACACGAGAAGTACGGAATCGAATCTTTTGCACTGATTCACGACTCCTTCGGTACCATTCCGGCTGACGCTGCGAACCTGTTCAAAGCA
GTGCGCGAAACTATGGTTGACACATATGAGTCTTGTGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCTTAGAGTCGGACTTCGCGTTCGCGTAA
>SEQ_ID_NO:_13_Tyr1_from_B._megaterium-protein_sequence
MGNKYRVRKNVLHLTDTEKRDFVRTVLILKEKGIYDRYIAWHGAAGKFHTPPGSDRNAAHMSSAFLPWHREYLLRFERDLQSINPEVTLP
YWEWETDAQMQDPSQSQIWSADFMGGNGNPIKDFIVDTGPFAAGRWTTIDEQGNPSGGLKRNFGATKEAPTLPTRDDVLNALKITQYDTP
PWDMTSQNSFRNQLEGFINGPQLHNRVHRWVGGQMGVVPTAPNDPVFFLHHANVDRIWAVWQIIHRNQNYQPMKNGPFGQNFRDPMYPWN
TTPEDVMNHRKLGYVYDIELRKSKRSS*
>SEQ_ID_NO:_14_Mel_from_S._antibioticus
MTVRKNQASLTAEEKRRFVAALLELKRTGRYDAFVTTHNAFILGDTDNGERTGHRSPSFLPWHRRFLLEFERALQSVDASVALPYWDWSA
DRSTRSSLWAPDFLGGTGRSRDGQVMDGPFAASAGNWPINVRVDGRTFLRRALGAGVSELPTRAEVDSVLAMATYDMAPWNSGSDGFRNH
LEGWRGVNLHNRVHVWVGGQMATGVSPNDPVFWLHHAYIDKLWAEWQRRHPSSPYLPGGGTPNVVDLNETMKPWNDTTPAALLDETRHYT
FDV
>SEQ_ID_NO:_15_Mel_from_R._etli
MAWLVGKPSLERSWNAILSFPESGFQLECRNTIGSSVFSSHFTLHFRVARRLLHFSCRRFTETQKEPTQALWWCELPTAPAPRRRGTGLK
AALILAKDNSNPRESKMSITRRHVIVQGGVIAAGLLASGLPGTKAFAQIPSIPWRRSLQGLAWNDPIIETYRDAVRLLNALPASDKFNWV
NLSKIHGSGDVVKYCPHGNWYFLPWHRAYTAMYERIVRHVIKNNDFAMPFWDWTDNPYLPEVFTMQKTPDGKDNPLYVSSRTWPITQPMP
DNIVGPQVLNTILTAKPYEVFGTTRPEGQNSLDPSWVTTSSGTQGALEYTPHNQVHNNIGGWMPEMSSPRDPIFFMHHCNIDRIWATWNL
RNANSTDRLWADMPFTDNFYDVDGNFWSPKVSDLYVPEELGYNYGFRTYFKVAAASAKTLALNDKLTSVIAATATDAAIAGVTTTSTDNS
KAATENVPLSLPIKIPAGALQEIVRQPPLPSGMDTMDFGAAQEQAASAPRVLAFLRDVEITSASTTSVRVFLGKNDLKADTPVTGPHYVG
SFAVLGHDGDEHRKPSFVLDLTDAIQRVYGGRGQTDGEAIDLQLIPVGSGAGKPGAVEPAKLEIAIVSA*
>SEQ_ID_NO:_16_Anderson_promoter_J23104
ttgacagctagctcagtcctaggtattgtgctagc
>SEQ_ID_NO:_17_RBS_B0034
ggatcttagctactagagaaagaggagaaatactag
>SEQ_ID_NO:_18_Pbad_promoter
TAGCATTTTTATCCATAAGATTAGCGGATCCTACCTGACGCTTTTTATCGCAACTCTCTACTGTTTCTCCATACCCGTTTTTTTGGAAGG
AGGTAAAGATCT
>SEQ_ID_NO:_19_pLux
acctgtaggatcgtacaggtttacgcaagaaaatggtttgttatagtcgaataaa
>SEQ_ID_NO:_20_pTet
tccctatcagtgatagagattgacatccctatcagtgatagagatactgagcac
>SEQ_ID_NO:_21_pLacI
tggtgcaaaacctttcgcggtatggcatgatagcgcc
>SEQ_ID_NO:_22_araC
atggctgaagcgcaaaatgatcccctgctgccgggatactcgtttaatgcccatctggtggcgggtttaacgccgattgaggccaacggt
tatctcgatttttttatcgaccgaccgctgggaatgaaaggttatattctcaatctcaccattcgcggtcagggggtggtgaaaaatcag
ggacgagaatttgtttgccgaccgggtgatattttgctgttcccgccaggagagattcatcactacggtcgtcatccggaggctcgcgaa
tggtatcaccagtgggtttactttcgtccgcgcgcctactggcatgaatggcttaactggccgtcaatatttgccaatacggggttcttt
cgcccggatgaagcgcaccagccgcatttcagcgacctgtttgggcaaatcattaacgccgggcaaggggaagggcgctattcggagctg
ctggcgataaatctgcttgagcaattgttactgcggcgcatggaagcgattaacgagtcgctccatccaccgatggataatcgggtacgc
gaggcttgtcagtacatcagcgatcacctggcagacagcaattttgatatcgccagcgtcgcacagcatgtttgcttgtcgccgtcgcgt
ctgtcacatcttttccgccagcagttagggattagcgtcttaagctggcgcgaggaccaacgtatcagccaggcgaagctgcttttgagc
accacccggatgcctatcgccaccgtcggtcgcaatgttggttttgacgatcaactctatttctcgcgggtatttaaaaaatgcaccggg
gccagcccgagcgagttccgtgccggttgtgaagaaaaagtgaatgatgtagccgtcaagttgtcataa
>SEQ_ID_NO:_23_LuxR
atgaaaaacataaatgccgacgacacatacagaataattaataaaattaaagcttgtagaagcaataatgatattaatcaatgcttatct
gatatgactaaaatggtacattgtgaatattatttactcgcgatcatttatcctcattctatggttaaatctgatatttcaatcctagat
aattaccctaaaaaatggaggcaatattatgatgacgctaatttaataaaatatgatcctatagtagattattctaactccaatcattca
ccaattaattggaatatatttgaaaacaatgctgtaaataaaaaatctccaaatgtaattaaagaagcgaaaacatcaggtcttatcact
gggtttagtttccctattcatacggctaacaatggcttcggaatgcttagttttgcacattcagaaaaagacaactatatagatagttta
tttttacatgcgtgtatgaacataccattaattgttccttctctagttgataattatcgaaaaataaatatagcaaataataaatcaaac
aacgatttaaccaaaagagaaaaagaatgtttagcgtgggcatgcgaaggaaaaagctcttgggatatttcaaaaatattaggttgcagt
gagcgtactgtcactttccatttaaccaatgcgcaaatgaaactcaatacaacaaaccgctgccaaagtatttctaaagcaattttaaca
ggagcaattgattgcccatactttaaaaattaa
>SEQ_ID_NO:_24_TetR
ATGTCCAGATTAGATAAAAGTAAAGTGATTAACAGCGCATTAGAGCTGCTTAATGAGGTCGGAATCGAAGGTTTAACAACCCGTAAACTC
GCCCAGAAGCTAGGTGTAGAGCAGCCTACATTGTATTGGCATGTAAAAAATAAGCGGGCTTTGCTCGACGCCTTAGCCATTGAGATGTTA
GATAGGCACCATACTCACTTTTGCCCTTTAGAAGGGGAAAGCTGGCAAGATTTTTTACGTAATAACGCTAAAAGTTTTAGATGTGCTTTA
CTAAGTCATCGCGATGGAGCAAAAGTACATTTAGGTACACGGCCTACAGAAAAACAGTATGAAACTCTCGAAAATCAATTAGCCTTTTTA
TGCCAACAAGGTTTTTCACTAGAGAATGCATTATATGCACTCAGCGCTGTGGGGCATTTTACTTTAGGTTGCGTATTGGAAGATCAAGAG
CATCAAGTCGCTAAAGAAGAAAGGGAAACACCTACTACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAAAACAACTTAAATGTGAAAGTGGGTCCTAA
>SEQ_ID_NO:_25_LacI
atgAAACCAGTAACGTTATACGATGTCGCAGAGTATGCCGGTGTCTCTTATATGACCGTTTCCCGCGTGGTGAACCAGGCCAGCCACGTT
TCTGCGAAAACGCGGGAAAAAGTGGAAGCGGCGATGGTGGAGCTGAATTACATTCCCAACCGCGTGGCACAACAACTGGCGGGCAAACAG
TCGTTGCTGATTGGCGTTGCCACCTCCAGTCTGGCCCTGCACGCGCCGTCGCAAATTGTCGCGGCGATTAAATCTCGCGCCGATCAACTG
GGTGCCAGCGTGGTGGTGTCGATGGTAGAACGAAGCGGCGTCGAAGCCTGTAAAGCGGCGGTGCACAATCTTCTCGCGCAACGCGTCAGT
GGGCTGATCATTAACTATCCGCTGGATGACCAGGATGCCATTGCTGTGGAAGCTGCCTGCACTAATGTTCCGGCGTTATTTCTTGATGTC
TCTGACCAGACACCCATCAACAGTATTATTTACTCCCATGAGGACGGTACGCGACTGGGCGTGGAGCATCTGGTCGCATTGGGTCACCAG
CAAATCGCGCTGTTAGCGGGCCCATTAAGTTCTGTCTCGGCGCGTCTGCGTCTGGCTGGCTGGCATAAATATCTCACTCGCAATCAAATT
CAGCCGATAGCGGAACGGGAAGGCGACTGGAGTGCCATGTCCGGTTTTCAACAAACCATGCAAATGCTGAATGAGGGCATCGTTCCCACT
GCGATGCTGGTTGCCAACGATCAGATGGCGCTGGGCGCAATGCGCGCCATTACCGAGTCCGGGCTGCGCGTTGGTGCGGATATCTCGGTA
GTGGGATACGACGATACCGAAGATAGCTCATGTTATATCCCGCCGTTAACCACCATCAAACAGGATTTTCGCCTGCTGGGGCAAACCAGC
GTGGACCGCTTGCTGCAACTCTCTCAGGGCCAGGCGGTGAAGGGCAATCAGCTGTTGCCAGTCTCACTGGTGAAAAGAAAAACCACCCTG
GCGCCCAATACGCAAACCGCCTCTCCCCGCGCGTTGGCCGATTCATTAATGCAGCTGGCACGACAGGTTTCCCGACTGGAAAGCGGGCAG
TGa
>SEQ_ID_NO:_26_first_half_of_69_T7RNAP_DNA
ATGAACACGATTAACATCGCTAAGAACGACTTCTCTGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAGCACGCTTCCGCAAGATGTTTGAGCGTCAACTTAAA
GCTGGTGAGGTTGCGGATAACGCTGCC
>SEQ_ID_NO:_27_first_half_of_69_T7RNAP_AA
MNTINIAKNDFSDIELAAIPFNTLADHYGERLAREQLALEHESYEMGEARFRKMFERQLKAGEVADNAA
>SEQ_ID_NO:_28_first_half_of_69_T7RNAP_+_nMag_DNA
ATGAACACGATTAACATCGCTAAGAACGACTTCTCTGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAGCACGCTTCCGCAAGATGTTTGAGCGTCAACTTAAA
GCTGGTGAGGTTGCGGATAACGCTGCCggcggtTCTggaggtCACACTCTTTACGCCCCTGGAGGATACGACATTATGGGATATTTGGAT
CAGATTGGGAACCGCCCAAACCCTCAGGTCGAACTGGGGCCTGTGGACACGTCATGTGCCCTGATCCTGTGCGATCTGAAGCAAAAGGAC
ACTCCGATCGTCTACGCCTCGGAAGCCTTCTTGTATATGACCGGATACAGCAATGCAGAGGTGCTCGGCAGGAACTGCAGATTCCTGCAG
TCCCCCGACGGGATGGTGAAACCAAAGTCGACTCGCAAATATGTGGACTCGAACACGATCAACACCATCCGGAAGGCCATCGACCGGAAC
GCCGAGGTCCAGGTGGAGGTGGTCAACTTTAAGAAGAACGGCCAGCGGTTCGTGAACTTTCTGACCATCATTCCGGTCCGGGATGAAACC
GGAGAGTACAGATACICCATGGGATTCCAGTGCGAAACCGAA
>SEQ_ID_NO:_29_first_half_of_69_T7RNAP_+_nMag_AA
MNTINIAKNDFSDIELAAIPFNTLADHYGERLAREQLALEHESYEMGEARFRKMFERQLKAGEVADNAAGGSGGHTLYAPGGYDIMGYLD
QIGNRPNPQVELGPVDTSCALILCDLKQKDTPIVYASEAFLYMTGYSNAEVLGRNCRFLQSPDGMVKPKSTRKYVDSNTINTIRKAIDRN
AEVQVEVVNFKKNGQRFVNFLTIIPVRDETGEYRYSMGFQCETE
>SEQ_ID_NO:_30_pMag(F2)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGGTCCTGTGCGATCTGAAGCAAAAGGACACTCCGGTGGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAA
>SEQ_ID_NO:_31_pMag(F2)_AA
MHTLYAPGGYDIMGYLRQIRNRPNPQVELGPVDTSCALVLCDLKQKDTPVVYASEAFLYMTGYSNAEVLGRNCRFLQSPDGMVKPKSTRK
YVDSNTINTMRKAIDRNAEVQVEVVNFKKNGQRFVNFLTMIPVRDETGEYRYSMGFQCETE
>SEQ_ID_NO:_32_Second_half_of_69_T7RNAP_DNA
GCCAAGCCTCTCATCACTACCCTACTCCCTAAGATGATTGCACGCATCAACGACTGGTTTGAGGAAGTGAAAGCTAAGCGCGGCAAGCGC
CCGACAGCCTTCCAGTTCCTGCAAGAAATCAAGCCGGAAGCCGTAGCGTACATCACCATTAAGACCACTCTGGCTTGCCTAACCAGTGCT
GACAATACAACCGTTCAGGCTGTAGCAAGCGCAATCGGTCGGGCCATTGAGGACGAGGCTCGCTTCGGTCGTATCCGTGACCTTGAAGCT
AAGCACTTCAAGAAAAACGTTGAGGAACAACTCAACAAGCGCGTAGGGCACGTCTACAAGAAAGCATTTATGCAAGTTGTCGAGGCTGAC
ATGCTCTCTAAGGGTCTACTCGGTGGCGAGGCGTGGTCTTCGTGGCATAAGGAAGACTCTATTCATGTAGGAGTACGCTGCATCGAGATG
CTCATTGAGTCAACCGGAATGGTTAGCTTACACCGCCAAAATGCTGGCGTAGTAGGTCAAGACTCTGAGACTATCGAACTCGCACCTGAA
TACGCTGAGGCTATCGCAACCCGTGCAGGTGCGCTGGCTGGCATCTCTCCGATGTTCCAACCTTGCGTAGTTCCTCCTAAGCCGTGGACT
GGCATTACTGGTGGTGGCTATTGGGCTAACGGTCGTCGTCCTCTGGCGCTGGTGCGTACTCACAGTAAGAAAGCACTGATGCGCTACGAA
GACGTTTACATGCCTGAGGTGTACAAAGCGATTAACATTGCGCAAAACACCGCATGGAAAATCAACAAGAAAGTCCTAGCGGTCGCCAAC
GTAATCACCAAGTGGAAGCATTGTCCGGTCGAGGACATCCCTGCGATTGAGCGTGAAGAACTCCCGATGAAACCGGAAGACATCGACATG
AATCCTGAGGCTCTCACCGCGTGGAAACGTGCTGCCGCTGCTGTGTACCGCAAGGACAAGGCTCGCAAGTCTCGCCGTATCAGCCTTGAG
TTCATGCTTGAGCAAGCCAATAAGTTTGCTAACCATAAGGCCATCTGGTTCCCTTACAACATGGACTGGCGCGGTCGTGTTTACGCTGTG
TCAATGTTCAACCCGCAAGGTAACGATATGACCAAAGGACTGCTTACGCTGGCGAAAGGTAAACCAATCGGTAAGGAAGGTTACTACTGG
CTGAAAATCCACGGTGCAAACTGTGCGGGTGTCGATAAGGTTCCGTTCCCTGAGCGCATCAAGTTCATTGAGGAAAACCACGAGAACATC
ATGGCTTGCGCTAAGTCTCCACTGGAGAACACTTGGTGGGCTGAGCAAGATTCTCCGTTCTGCTTCCTTGCGTTCTGCTTTGAGTACGCT
GGGGTACAGCACCACGGCCTGAGCTATAACTGCTCCCTTCCGCTGGCGTTTGACGGGTCTTGCTCTGGCATCCAGCACTTCTCCGCGATG
CTCCGAGATGAGGTAGGTGGTCGCGCGGTTAACTTGCTTCCTAGTGAAACCGTTCAGGACATCTACGGGATTGTTGCTAAGAAAGTCAAC
GAGATTCTACAAGCAGACGCAATCAATGGGACCGATAACGAAGTAGTTACCGTGACCGATGAGAACACTGGTGAAATCTCTGAGAAAGTC
AAGCTGGGCACTAAGGCACTGGCTGGTCAATGGCTGGCTTACGGTGTTACTCGCAGTGTGACTAAGAGTTCAGTCATGACGCTGGCTTAC
GGGTCCAAAGAGTTCGGCTTCCGTCAACAAGTGCTGGAAGATACCATTCAGCCAGCTATTGATTCCGGCAAGGGTCTGATGTTCACTCAG
CCGAATCAGGCTGCTGGATACATGGCTAAGCTGATTTGGGAATCTGTGAGCGTGACGGTGGTAGCTGCGGTTGAAGCAATGAACTGGCTT
AAGTCTGCTGCTAAGCTGCTGGCTGCTGAGGTCAAAGATAAGAAGACTGGAGAGATTCTTCGCAAGCGTTGCGCTGTGCATTGGGTAACT
CCTGATGGTTTCCCTGTGTGGCAGGAATACAAGAAGCCTATTCAGACGCGCTTGAACCTGATGTTCCTCGGTCAGTTCCGCTTACAGCCT
ACCATTAACACCAACAAAGATAGCGAGATTGATGCACACAAACAGGAGTCTGGTATCGCTCCTAACTTTGTACACAGCCAAGACGGTAGC
CACCTTCGTAAGACTGTAGTGTGGGCACACGAGAAGTACGGAATCGAATCTTTTGCACTGATTCACGACTCCTTCGGTACCATTCCGGCT
GACGCTGCGAACCTGTTCAAAGCAGTGCGCGAAACTATGGTTGACACATATGAGTCTTGTGATGTACTGGCTGATTTCTACGACCAGTTC
GCTGACCAGTTGCACGAGTCTCAATTGGACAAAATGCCAGCACTTCCGGCTAAAGGTAACTTGAACCTCCGTGACATCTTAGAGTCGGAC
TTCGCGTTCGCGTAA
>SEQ_ID_NO:_33_Second_half_of_69_T7RNAP_AA
AKPLITTLLPKMIARINDWFEEVKAKRGKRPTAFQFLQEIKPEAVAYITIKTTLACLTSADNTTVQAVASAIGRAIEDEARFGRIRDLEA
KHEKKNVEEQUNKRVGHVYKKAFMQVVEADMISKGLLGGEAWSSWHKEDSIHVGVRCIEMLIESTGMVSLHRQNAGVVGQDSETIELAPE
YAEAIATRAGALAGISPMFQPCVVPPKPWTGITGGGYWANGRRPLALVRTHSKKALMRYEDVYMPEVYKAINIAQNTAWKINKKVLAVAN
VITKWKHCPVEDIPAIEREELPMKPEDIDMNPEALTAWKRAAAAVYRKDKARKSRRISLEFMLEQANKFANHKAIWFPYNMDWRGRVYAV
SMFNPQGNDMTKGLLTLAKGKPIGKEGYYWLKIHGANCAGVDKVPFPERIKFIEENHENIMACAKSPLENTWWAEQDSPFCFLAFCFEYA
GVQHHGLSYNCSLPLAFDGSCSGIQHFSAMLRDEVGGRAVNLLPSETVQDIYGIVAKKVNEILQADAINGTDNEVVTVTDENTGEISEKV
KLGTKALAGQWLAYGVTRSVIKSSVMTLAYGSKEFGFRQQVLEDTIQPAIDSGKGLMFTQPNQAAGYMAKLIWESVSVTVVAAVEAMNWL
KSAAKLLAAEVKDKKTGEILRKRCAVHWVTPDGEPVWQEYKKPIQTRLNLMFLGQFRLQPTINTNKDSEIDAHKQESGIAPNEVESQDGS
ELRKTVVWAHEKYGIESFALIHDSFGTIPADAANLFKAVRETMVDTYESCDVLADFYDQFADQLHESQLDKMPALPAKGNINLRDILESD
FAFA
>SEQ_ID_NO:_34_second_half_of_563_T7RNAP_non-star_variant_DNA
AGTGAAACCGTTCAGGACATCTACGGGATTGTTGCTAAGAAAGTCAACGAGATTCTACAAGCAGACGCAATCAATGGGACCGATAACGAA
GTAGTTACCGTGACCGATGAGAACACTGGTGAAATCTCTGAGAAAGTCAAGCTGGGCACTAAGGCACTGGCTGGTCAATGGCTGGCTTAC
GGTGTTACTCGCAGTGTGACTAAGCGTTCAGTCATGACGCTGGCTTACGGGTCCAAAGAGTTCGGCTTCCGTCAACAAGTGCTGGAAGAT
ACCATTCAGCCAGCTATTGATTCCGGCAAGGGTCTGATGTTCACTCAGCCGAATCAGGCTGCTGGATACATGGCTAAGCTGATTTGGGAA
TCTGTGAGCGTGACGGTGGTAGCTGCGGTTGAAGCAATGAACTGGCTTAAGTCTGCTGCTAAGCTGCTGGCTGCTGAGGTCAAAGATAAG
AAGACTGGAGAGATTCTTCGCAAGCGTTGCGCTGTGCATTGGGTAACTCCTGATGGTTTCCCTGTGTGGCAGGAATACAAGAAGCCTATT
CAGACGCGCTTGAACCTGATGTTCCTCGGTCAGTTCCGCTTACAGCCTACCATTAACACCAACAAAGATAGCGAGATTGATGCACACAAA
CAGGAGTCTGGTATCGCTCCTAACTTTGTACACAGCCAAGACGGTAGCCACCTTCGTAAGACTGTAGTGTGGGCACACGAGAAGTACGGA
ATCGAATCTTTTGCACTGATTCACGACTCCTTCGGTACCATTCCGGCTGACGCTGCGAACCTGTTCAAAGCAGTGCGCGAAACTATGGTT
GACACATATGAGTCTTGTGATGTACTGGCTGATTTCTACGACCAGTTCGCTGACCAGTTGCACGAGTCTCAATTGGACAAAATGCCAGCA
CTTCCGGCTAAAGGTAACTTGAACCTCCGTGACATCTTAGAGTCGGACTTCGCGTTCGCG
>SEQ_ID_NO:_35_second_half_of_563_T7RNAP_non-star_variant_AA
SETVQDIYGIVAKKVNEILQADAINGTDNEVVTVTDENTGEISEKVKLGTKALAGQWLAYGVTRSVTKRSVMTLAYGSKEFGFRQQVLED
TIQPAIDSGKGLMFTQPNQAAGYMAKLIWESVSVTVVAAVEAMNWLKSAAKLLAAEVKDKKTGEILRKRCAVHWVTPDGFPVWQEYKKPI
QTRLNLMFLGQFRLQPTINTNKDSEIDAHKQESGIAPNFVESQDGSHLRKTVVWAHEKYGIESFALIHDSFGTIPADAANLFKAVRETMV
DTYESCDVLADFYDQFADQLHESQLDKMPALPAKGNLNLRDILESDFAFA
>SEQ_ID_NO:_36_second_half_of_563_T7RNAP_+_pMag_(F2)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGGTCCTGTGCGATCTGAAGCAAAAGGACACTCCGGTGGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAAggcggtTCTggaggtAGTGAAACCGTTCAGGACATCTACGGGATTGTTGCTAAGAAAGTCAACGAGATTCTACAAGCAGACGCAATC
AATGGGACCGATAACGAAGTAGTTACCGTGACCGATGAGAACACTGGTGAAATCTCTGAGAAAGTCAAGCTGGGCACTAAGGCACTGGCT
GGTCAATGGCTGGCTTACGGTGTTACTCGCAGTGTGACTAAGaGTTCAGTCATGACGCTGGCTTACGGGTCCAAAGAGTTCGGCTTCCGT
CAACAAGTGCTGGAAGATACCATTCAGCCAGCTATTGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAATGAACTGGCTTAAGTCTGCTGCTAAGCTGCTGGCT
GCTGAGGTCAAAGATAAGAAGACTGGAGAGATTCTTCGCAAGCGTTGCGCTGTGCATTGGGTAACTCCTGATGGTTTCCCTGTGTGGCAG
GAATACAAGAAGCCTATTCAGACGCGCTTGAACCTGATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCCAAGACGGTAGCCACCTTCGTAAGACTGTAGTGTGG
GCACACGAGAAGTACGGAATCGAATCTTTTGCACTGATTCACGACTCCTTCGGTACCATTCCGGCTGACGCTGCGAACCTGTTCAAAGCA
GTGCGCGAAACTATGGTTGACACATATGAGTCTTGTGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCTTAGAGTCGGACTTCGCGTTCGCG
>SEQ_ID_NO:_37_second_half_of_563_T7RNAP_+_pMag_(F2)_AA
MHTLYAPGGYDIMGYLRQIRNRPNPQVELGPVDISCALVLCDLKQKDTPVVYASEAFLYMIGYSNAEVLGRNCRFLQSPDGMVKPKSTRK
YVDSNTINTMRKAIDRNAEVQVEVVNFKKNGQREVNELTMIPVRDETGEYRYSMGFQCETEGGSGGSETVQDIYGIVAKKVNEILQADAI
NGTDNEVVTVTDENTGEISEKVKIGTKALAGQWLAYGVTRSVTKSSVMTLAYGSKEFGFRQQVLEDTIQPAIDSGKGIMETQPNQAAGYM
AKLIWESVSVTVVAAVEAMNWIKSAAKLLAAEVKDKKTGEILRKRCAVHWVTPDGFPVWQEYKKPIQTRLNLMELGQFRLQPTINTNKDS
EIDAHKQESGIAPNFVHSQDGSHLRKTVVWAHEKYGIESFALIHDSFGTIPADAANLFKAVRETMVDTYESCDVLADFYDQFADQLHESQ
LDKMPALPAKGNLNLRDILESDFAFA
>SEQ_ID_NO:_38_Second_half_of_69_T7RNAP_+_pMag_(Standard)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGATCCTGTGCGATCTGAAGCAAAAGGACACTCCGATCGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAAggcggtTCTggaggtGCCAAGCCTCTCATCACTACCCTACTCCCTAAGATGATTGCACGCATCAACGACTGGTTTGAGGAAGTGAAA
GCTAAGCGCGGCAAGCGCCCGACAGCCTTCCAGTTCCTGCAAGAAATCAAGCCGGAAGCCGTAGCGTACATCACCATTAAGACCACTCTG
GCTTGCCTAACCAGTGCTGACAATACAACCGTTCAGGCTGTAGCAAGCGCAATCGGTCGGGCCATTGAGGACGAGGCTCGCTTCGGTCGT
ATCCGTGACCTTGAAGCTAAGCACTTCAAGAAAAACGTTGAGGAACAACTCAACAAGCGCGTAGGGCACGTCTACAAGAAAGCATTTATG
CAAGTTGTCGAGGCTGACATGCTCTCTAAGGGTCTACTCGGTGGCGAGGCGTGGTCTTCGTGGCATAAGGAAGACTCTATTCATGTAGGA
GTACGCTGCATCGAGATGCTCATTGAGTCAACCGGAATGGTTAGCTTACACCGCCAAAATGCTGGCGTAGTAGGTCAAGACTCTGAGACT
ATCGAACTCGCACCTGAATACGCTGAGGCTATCGCAACCCGTGCAGGTGCGCTGGCTGGCATCTCTCCGATGTTCCAACCTTGCGTAGTT
CCTCCTAAGCCGTGGACTGGCATTACTGGTGGTGGCTATTGGGCTAACGGTCGTCGTCCTCTGGCGCTGGTGCGTACTCACAGTAAGAAA
GCACTGATGCGCTACGAAGACGTTTACATGCCTGAGGTGTACAAAGCGATTAACATTGCGCAAAACACCGCATGGAAAATCAACAAGAAA
GTCCTAGCGGTCGCCAACGTAATCACCAAGTGGAAGCATTGTCCGGTCGAGGACATCCCTGCGATTGAGCGTGAAGAACTCCCGATGAAA
CCGGAAGACATCGACATGAATCCTGAGGCTCTCACCGCGTGGAAACGTGCTGCCGCTGCTGTGTACCGCAAGGACAAGGCTCGCAAGTCT
CGCCGTATCAGCCTTGAGTTCATGCTTGAGCAAGCCAATAAGTTTGCTAACCATAAGGCCATCTGGTTCCCTTACAACATGGACTGGCGC
GGTCGTGTTTACGCTGTGTCAATGTTCAACCCGCAAGGTAACGATATGACCAAAGGACTGCTTACGCTGGCGAAAGGTAAACCAATCGGT
AAGGAAGGTTACTACTGGCTGAAAATCCACGGTGCAAACTGTGCGGGTGTCGATAAGGTTCCGTTCCCTGAGCGCATCAAGTTCATTGAG
GAAAACCACGAGAACATCATGGCTTGCGCTAAGTCTCCACTGGAGAACACTTGGTGGGCTGAGCAAGATTCTCCGTTCTGCTTCCTTGCG
TTCTGCTTTGAGTACGCTGGGGTACAGCACCACGGCCTGAGCTATAACTGCTCCCTTCCGCTGGCGTTTGACGGGTCTTGCTCTGGCATC
CAGCACTTCTCCGCGATGCTCCGAGATGAGGTAGGTGGTCGCGCGGTTAACTTGCTTCCTAGTGAAACCGTTCAGGACATCTACGGGATT
GTTGCTAAGAAAGTCAACGAGATTCTACAAGCAGACGCAATCAATGGGACCGATAACGAAGTAGTTACCGTGACCGATGAGAACACTGGT
GAAATCTCTGAGAAAGTCAAGCTGGGCACTAAGGCACTGGCTGGTCAATGGCTGGCTTACGGTGTTACTCGCAGTGTGACTAAGAGTTCA
GTCATGACGCTGGCTTACGGGTCCAAAGAGTTCGGCTTCCGTCAACAAGTGCTGGAAGATACCATTCAGCCAGCTATTGATTCCGGCAAG
GGTCTGATGTTCACTCAGCCGAATCAGGCTGCTGGATACATGGCTAAGCTGATTTGGGAATCTGTGAGCGTGACGGTGGTAGCTGCGGTT
GAAGCAATGAACTGGCTTAAGTCTGCTGCTAAGCTGCTGGCTGCTGAGGTCAAAGATAAGAAGACTGGAGAGATTCTTCGCAAGCGTTGC
GCTGTGCATTGGGTAACTCCTGATGGTTTCCCTGTGTGGCAGGAATACAAGAAGCCTATTCAGACGCGCTTGAACCTGATGTTCCTCGGT
CAGTTCCGCTTACAGCCTACCATTAACACCAACAAAGATAGCGAGATTGATGCACACAAACAGGAGTCTGGTATCGCTCCTAACTTTGTA
CACAGCCAAGACGGTAGCCACCTTCGTAAGACTGTAGTGTGGGCACACGAGAAGTACGGAATCGAATCTTTTGCACTGATTCACGACTCC
TTCGGTACCATTCCGGCTGACGCTGCGAACCTGTTCAAAGCAGTGCGCGAAACTATGGTTGACACATATGAGTCTTGTGATGTACTGGCT
GATTTCTACGACCAGTTCGCTGACCAGTTGCACGAGTCTCAATTGGACAAAATGCCAGCACTTCCGGCTAAAGGTAACTTGAACCTCCGT
GACATCTTAGAGTCGGACTTCGCGTTCGCGTAA
>SEQ_ID_NO:_39_Second_half_of_69_T7RNAP_+_pMag_(Standard)_AA
MHTLYAPGGYDIMGYLRQIRNRPNPQVELGPVDISCALILCDLKQKDTPIVYASEAFLYMIGYSNAEVLGRNCRFLQSPDGMVKPKSTRK
YVDSNTINTMRKAIDRNAEVQVEVVNFKKNGQRFVNELTMIPVRDETGEYRYSMGFQCETEGGSGGAKPLITTLLPKMIARINDWFEEVK
AKRGKRPTAFQFLQEIKPEAVAYITIKTTLACLTSADNTTVQAVASAIGRAIEDEARFGRIRDLEAKHFKKNVEEQLNKRVGHVYKKAFM
QVVEADMISKGLIGGEAWSSWHKEDSIHVGVRCIEMLIESTGMVSLHRQNAGVVGQDSETIELAPEYAEAIATRAGALAGISPMFQPCVV
PPKPWTGITGGGYWANGRRPLALVRTHSKKALMRYEDVYMPEVYKAINIAQNTAWKINKKVLAVANVITKWKHCPVEDIPAIEREELPMK
PEDIDMNPEALTAWKRAAAAVYRKDKARKSRRISLEFMLEQANKFANHKAIWFPYNMDWRGRVYAVSMFNPQGNDMTKGLLTLAKGKPIG
KEGYYWLKIHGANCAGVDKVPFPERIKFIEENHENIMACAKSPLENTWWAEQDSPFCFLAFCFEYAGVQHEGLSYNCSLPLAFDGSCSGI
QHFSAMLRDEVGGRAVNLLPSETVQDIYGIVAKKVNEILQADAINGTDNEVVTVTDENTGEISEKVKLGTKALAGQWLAYGVTRSVTKSS
VMTLAYGSKEFGFRQQVLEDTIQPAIDSGKGLMFTQPNQAAGYMAKLIWESVSVTVVAAVEAMNWLKSAAKLLAAEVKDKKTGEILRKRC
AVHWVIPDGFPVWQEYKKPIQTRINLMELGQFRLQPTINTNKDSEIDAHKQESGIAPNEVESQDGSHLRKTVVWAHEKYGIESFALIHDS
FGTIPADAANLFKAVRETMVDTYESCDVLADFYDQFADQLHESQLDKMPALPAKGNLNLRDILESDEAFA*
>SEQ_ID_NO:_40_second_half_of_563_17RNAP_+_pMag_(standard)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGATCCTGTGCGATCTGAAGCAAAAGGACACTCCGATCGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAAggcggtTCTggaggtAGTGAAACCGTTCAGGACATCTACGGGATTGTTGCTAAGAAAGTCAACGAGATTCTACAAGCAGACGCAATC
AATGGGACCGATAACGAAGTAGTTACCGTGACCGATGAGAACACTGGTGAAATCTCTGAGAAAGTCAAGCTGGGCACTAAGGCACTGGCT
GGTCAATGGCTGGCTTACGGTGTTACTCGCAGTGTGACTAAGaGTTCAGTCATGACGCTGGCTTACGGGTCCAAAGAGTTCGGCTTCCGT
CAACAAGTGCTGGAAGATACCATTCAGCCAGCTATTGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAATGAACTGGCTTAAGTCTGCTGCTAAGCTGCTGGCT
GCTGAGGTCAAAGATAAGAAGACTGGAGAGATTCTTCGCAAGCGTTGCGCTGTGCATTGGGTAACTCCTGATGGTTTCCCTGTGTGGCAG
GAATACAAGAAGCCTATTCAGACGCGCTTGAACCTGATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCCAAGACGGTAGCCACCTTCGTAAGACTGTAGTGTGG
GCACACGAGAAGTACGGAATCGAATCTTTTGCACTGATTCACGACTCCTTCGGTACCATTCCGGCTGACGCTGCGAACCTGTTCAAAGCA
GTGCGCGAAACTATGGTTGACACATATGAGTCTTGTGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCTTAGAGTCGGACTTCGCGTTCGCG
>SEQ_ID_NO:_41_second_half_of_563_T7RNAP_+_pMag_(standard)_AA
MHTLYAPGGYDIMGYLRQIRNRPNPQVELGPVDTSCALILCDLKQKDTPIVYASEAFLYMTGYSNAEVLGRNCRFLQSPDGMVKPKSTRK
YVDSNTINTMRKAIDRNAEVQVEVVNFKKNGQREVNFLTMIPVRDETGEYRYSMGFQCETEGGSGGSETVQDIYGIVAKKVNEILQADAI
NGTDNEVVTVTDENTGEISEKVKLGTKALAGQWLAYGVTRSVTKSSVMTLAYGSKEFGFRQQVLEDTIQPAIDSGKGLMFTQPNQAAGYM
AKLIWESVSVTVVAAVEAMNWLKSAAKLLAAEVKDKKTGEILRKRCAVHWVTPDGFPVWQEYKKPIQTRLNLMFLGQFRLQPTINTNKDS
EIDAHKQESGIAPNFVHSQDGSHLRKTVVWAHEKYGIESFALIHDSFGTIPADAANLFKAVRETMVDTYESCDVLADFYDQFADQLHESQ
LDKMPALPAKGNINIRDILESDFAFA
>SEQ_ID_NO:_42_second_half_of_563_T7RNAP_non-star_variant_+_pMag_(F1)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGaTCCTGTGCGATCTGAAGCAAAAGGACACTCCGGTGGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAAggcggtTCTggaggtAGTGAAACCGTTCAGGACATCTACGGGATTGTTGCTAAGAAAGTCAACGAGATTCTACAAGCAGACGCAATC
AATGGGACCGATAACGAAGTAGTTACCGTGACCGATGAGAACACTGGTGAAATCTCTGAGAAAGTCAAGCTGGGCACTAAGGCACTGGCT
GGTCAATGGCTGGCTTACGGTGTTACTCGCAGTGTGACTAAGCGTTCAGTCATGACGCTGGCTTACGGGTCCAAAGAGTTCGGCTTCCGT
CAACAAGTGCTGGAAGATACCATTCAGCCAGCTATTGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAATGAACTGGCTTAAGTCTGCTGCTAAGCTGCTGGCT
GCTGAGGTCAAAGATAAGAAGACTGGAGAGATTCTTCGCAAGCGTTGCGCTGTGCATTGGGTAACTCCTGATGGTTTCCCTGTGTGGCAG
GAATACAAGAAGCCTATTCAGACGCGCTTGAACCTGATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCCAAGACGGTAGCCACCTTCGTAAGACTGTAGTGTGG
GCACACGAGAAGTACGGAATCGAATCTTTTGCACTGATTCACGACTCCTTCGGTACCATTCCGGCTGACGCTGCGAACCTGTTCAAAGCA
GTGCGCGAAACTATGGTTGACACATATGAGTCTTGTGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCTTAGAGTCGGACTTCGCGTTCGCG
>SEQ_ID_NO:_43_second_half_of_563_T7RNAP_non-star_variant_+_pMag_(F1)_AA
MHTLYAPGGYDIMGYLRQIRNRPNPQVELGPVDTSCALILCDLKQKDTPVVYASEAFLYMTGYSNAEVLGRNCRFLQSPDGMVKPKSTRK
YVDSNTINTMRKAIDRNAEVQVEVVNFKKNGQRFVNFLTMIPVRDETGEYRYSMGFQCETEGGSGGSETVQDIYGIVAKKVNEILQADAI
NGTDNEVVTVIDENTGEISEKVALGTKALAGQWLAYGVIRSVTKRSVMTLAYGSKEFGFRQQVLEDTIQPAIDSGKGLMFTQPNQAAGYM
AKLIWESVSVTVVAAVEAMNWIKSAAKLLAAEVKDKKTGEILRKRCAVHWVIPDGFPVWQEYKKPIQTRLNLMELGQFRLQPTININKDS
EIDAHKQESGIAPNFVHSQDGSHLRKTVVWAHEKYGIESFALIHDSEGTIPADAANLFKAVRETMVDTYESCDVLADFYDQFADQLHESQ
LDKMPALPAKGNINIRDILESDEAFA*
>SEQ_ID_NO:_44_pMag_(standard)_DNA
ATGCACACTCTTTACGCCCCTGGAGGATACGACATTATGGGATATTTGCGGCAGATTAGGAACCGCCCAAACCCTCAGGTCGAACTGGGG
CCTGTGGACACGTCATGTGCCCTGATCCTGTGCGATCTGAAGCAAAAGGACACTCCGATCGTCTACGCCTCGGAAGCCTTCTTGTATATG
ACCGGATACAGCAATGCAGAGGTGCTCGGCAGGAACTGCAGATTCCTGCAGTCCCCCGACGGGATGGTGAAACCAAAGTCGACTCGCAAA
TATGTGGACTCGAACACGATCAACACCATGCGGAAGGCCATCGACCGGAACGCCGAGGTCCAGGTGGAGGTGGTCAACTTTAAGAAGAAC
GGCCAGCGGTTCGTGAACTTTCTGACCATGATTCCGGTCCGGGATGAAACCGGAGAGTACAGATACTCCATGGGATTCCAGTGCGAAACC
GAA
>SEQ_ID_NO:_45__pMag_(standard)_AA
MHTLYAPGGYDIMGYLRQIRNRPNPQVELGPVDISCALILCDLKQKDTPIVYASEAFLYMIGYSNAEVLGRNCRFLQSPDGMVKPKSTRK
YVDSNTINTMRKAIDRNAEVQVEVVNFKKNGQRFVNFLTMIPVRDETGEYRYSMGFQCETE

Various embodiments and instances of aspects of the invention are as set out in the following numbered paragraphs.
1. A method for producing melanated bacterial cellulose, wherein the method comprises exposing a cellulose pellicle that comprises tyrosinase to a development solution, wherein the development solution:
   is at a pH of between 6 and 8.5; and
   comprises L-tyrosine and/or L-cysteine and/or L-cystine;
   and optionally comprises metal ions with an oxidation state of 2+;
optionally wherein the cellulose pellicle was produced by bacterial cells that express tyrosinase.
2. A method for producing melanated bacterial cellulose, wherein the method comprises:
a) culturing a cellulose producing bacteria under conditions so as to allow a pellicle 50 to form, wherein the bacteria express tyrosinase; and
b) exposing the pellicle formed in a) to a development solution; wherein the development solution:
   is at a pH of between 6 and 8.5; and
   comprises L-tyrosine and/or L-cysteine and/or L-cystine; and
   optionally comprises metal ions with an oxidation state of 2+.
3. The method according to paragraph 2 wherein the conditions that allow a pellicle to form comprise culturing the bacteria:
a) at a pH of:
   between 3-7, optionally a pH of between 3.25 and 6.75, 3.5 and 6.5, 3.5 and 6.25, 3.75 and 6, 4 and 5.75, 4.25 and 5.5, 4.5 and 5.25; pH 5.8; and/or
   at least 3 but less than or equal to pH 7, for example at least 3, 3.25, 3.5, 3.75, 4, 4.25, 4.5, 4.75, 5, 5.25, 5.5, 5.75, 5.8, 6, 6.25, 6.5, 6.75, but less than or equal to pH 7;
and/or
b) in culture media that is:
   i) HS media; or
   ii) Coconut water media.
4. The method according to any of paragraphs 2 or 3 wherein (b) is performed after the pellicle formed in (a) is harvested.
5. The method according to any of the preceding paragraphs wherein the tyrosinase is a bacterial tyrosinase, optionally is:
   i) Tyr1 from Bacillus megaterium [SEQ ID NO: 13];
   ii) mel from Streptomyces antibiotics [SEQ ID NO: 14]; or
   iii) mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
6. The method according to any of the preceding paragraphs wherein the bacterial cells are capable of producing bacterial cellulose.
7. The method according to any of the preceding paragraphs wherein the bacterial cells express all of bcsA, bcsD, bscC and bscD.
8. The method according to any of the preceding paragraphs wherein the bacterial cells belong to a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes.
9. The method according to any of the preceding paragraphs wherein the bacterial cells are selected from the group comprising or consisting of: Komagaeibacter rhaeticus; Komagaeibacter xylinus, Komagaeibacter hansenii, Komagaeibacter medellinensis, Komagaelbacter europaeus, Komagaeibacter maltaceti, Komagaeibacter pomaceti, Komagaeibacter oboediens, or Komagaeibacter saccharivoans.
10. The method according to any of the preceding paragraphs wherein the bacterial cells are selected from the group comprising or consisting of:
a) a strain of Komagaeibacter rhaeticus selected from the group comprising or consisting of: Komagaeibacter rhaeticus IGEM. Komagaelbacter rhaeticus AF1; Komagaelbacter rhaeticus LMG22126; or
b) Gluconacetobacter xylinus CGMCC 2995.
11. The method according to any of the preceding paragraphs wherein the bacterial cells are Komagaeibacter rhaeticus iGEM cells.
12. The method according to any of the preceding paragraphs wherein the metal ions with an oxidation state of 2+ are selected from:
a) Cu²⁺, Zn²⁺, Be²⁺, Mg²⁺, Ca²⁺, Cr²⁺, Mn²⁺, Co²⁺ or Ni²⁺;
b) Cu²⁺, Zn²⁺; or
c) Cu²+.
13. The method according to any of the preceding paragraphs wherein the development solution comprises a water-soluble copper (II) salt, optionally comprises CuSO₄, or CuCl₂ optionally comprises:
a) at least 2 μM CuSO₄, optionally at least 3 μM, 4 μM, 5 μM, 7.5 μM, 10 μM, 12.5 μM, 15 μM, 17.5 μM, 20 μM, 40 μM, 60 μM, 80 μM, 100 μM, 120 μM, 140 μM, or at least 160 μM CuSO₄; and/or
between 2 μM CuSO₄ and 20 μM CuSO₄; and/or
less than 20 μM, optionally less than 17.5 μM, 15 μM, 12.5 μM, 10 μM, 7.5 μM, 5 μM, 4 μM, 3 μM, or less than 2 μM;
and/or
between 20 μM and 160 μM CuSO₄; and/or
less than 160 μM, optionally less than 140 μM, 120 μM, 100 μM, 80 μM, 60 μM, 40 μM, or less than 20 μM CuSO₄;
and/or
b) at least 2 μM CuCl₂, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuCl₂; and/or
between 2 μM CuCl₂ and 20 μM CuCl₂; and/or
less than 20 μM, optionally less than 17.5 μM, 15 μM, 12.5 μM, 10 μM, 7.5 μM, 5 μM, 4 μM, 3 μM, or less than 2 μM; and/or
between 20 μM and 160 μM CuCl₂; and/or
less than 160 μM, optionally less than 140 μM, 120 μM, 100 μM, 80 μM, 60 μM, 40 μM, or less than 20 μM CuCl₂.
14. The method according to any of the preceding paragraphs wherein the development solution comprises:
   at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L;
   between 0.1 g/L and 2 g/L tyrosine; and/or
   less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine.
15. The method according to any of the preceding paragraphs wherein the development solution comprises:
a) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
   between 10 g/L and 300 g/L cysteine; and/or
   less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
and/or
b) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L;
   between 0.1 g/L and 2 g/L L-cystine; and/or
   less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine.
16. The method according to any of the preceding paragraphs wherein the development solution is at a pH of:
   at least 6, optionally at least 6.25, 6.5, 6.75, 7, 7.25, 7.4, 7.5, 7.75, 8, 8.25 or at least 8.5; and/or
   between 6 and 8.5, optionally between 6.25 and 8.25, 6.5 and 8, 6.25 and 7.75, 6.5 and 7.5, 6.75 and 7.25; or
   7.4.
17. The method according to any of the preceding paragraphs wherein the development solution comprises a buffer selected from the group comprising PBS, HEPES, MOPS and TRIS, optionally wherein the buffer is PBS.
18. The method according to any of the preceding paragraphs wherein the development solution comprises:
   a) PBS at pH 7.4;
   b) 10 μM CuSO₄ or 10 μM CuSO₄; and
   c) 0.5 g/L L-tyrosine or 1 g/L L-tyrosine, and/or 1 g/L L-cysteine and/or 0.4 g/L L-cystine.
19. The method according to any of the preceding paragraphs wherein the cellulose pellicle is incubated in the development solution at a temperature of:
   between 25° C. and 50° C., optionally between 30° C. and 45° C., 35° C. and 40° C.; and/or
   at least 25° C. optionally a at least 30° C., 32° C., 34° C., 36° C., 38° C., 40° C., 42° C., 44° C., 46° C., 48° C. or at least 50° C.; and/or
   30° C.; and/or
   45° C.
20. The method according to any of the preceding paragraphs wherein the cellulose pellicle is incubated in the development solution for:
   at least 1 hour, optionally at least 2 hours, 3, 4, 5, 6, 7, 8, 9, 10, 11, 12, 13, 14, 15, 16, 17, 18, 19, 20, 21, 22, 23, 24, 36, or at least 48 hours;
   between 1 hour and 48 hours, or between 2 and 36, 3 and 24, 4 and 23, 5 and 22, 6 and 21, 7 and 20, 8 and 19, 9 and 18, 10 and 17, 11 and 16, 12 and 15, 13 and 14 hours; and/or
for less than 48 hours, optionally less than 36 hours, 24, 23, 22, 21, 20, 19, 18, 17, 16, 15, 14, 13, 12, 11, 10, 9, 8, 7, 6, 5, 4, 3, 2, or less than 1 hour.
21. The method according to any of the preceding paragraphs wherein the method comprises a further step of:
(c) sterilising the pellicle following incubation in the development solution, optionally wherein the sterilisation is selected from the group comprising or consisting of:
   i) autoclaving;
   ii) heating; and/or
   iii) desiccation, optionally with 70% ethanol.
22. The method according to any of paragraphs 1-21 wherein the tyrosinase is a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
23. The method according to any of the preceding paragraphs wherein the tyrosinase gene is operably linked to Anderson promoter J23104 [SEQ ID NO: 16] and RBS B0034 [SEQ ID NO: 17];
optionally is operably linked to Anderson promoter J23104 and RBS B0034 that comprises or consists of a sequence with at least 80%, 82%, 84%, 86%, 88%, 90%, 92%, 94%, 96%, 98%, 99% or 100% sequence identity to any of the above sequences.
24. The method according to any of the preceding paragraphs wherein the cells are cultured in a culture medium that:
i) is Hestrin and Schramm (HS) medium;
ii) is supplemented with glucose, optionally at 2% (w/v); and/or
iii) is buffered to a pH of 5.8.
25. A nucleic acid comprising a regulatory sequence and a sequence that encodes a tyrosinase enzyme wherein the regulatory sequence comprises Anderson promoter J23104 and RBS B0034.
26. The nucleic acid according to paragraph 25 wherein the sequence that encodes a tyrosinase enzyme encodes a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
27. The nucleic acid according to paragraph 25 or 26 wherein the nucleic acid is a circular nucleic acid, optionally is a circular nucleic acid selected from the group consisting of a plasmid, a bacterial artificial chromosome, a phagemid, a cosmid, a yeast artificial chromosome, a human artificial chromosome, a viral vector.
28. The nucleic acid according to paragraph 27 wherein the circular nucleic acid, optionally a plasmid, further comprises an origin of replication.
29. The nucleic acid according any of the proceeding paragraphs wherein any one or more of the nucleic acids further comprises a selectable marker.
30. The nucleic acid according to any of paragraphs 25-29 wherein the nucleic acid is integrated into the genome of a cell, optionally a bacterial cell.
31. A cell comprising the nucleic acid according to any of 25-30 wherein the cell is:
   i) a bacterial cell that is capable of producing bacterial cellulose;
   ii) a bacterial cell that expresses all of bcsA, bcsD, bscC and bscD;
   iii) a bacterial cell of a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes;
   iv) a bacterial cell selected from the group comprising or consisting of: Komagaelbacter rhaeticus; Komagaelbacter xylinus, Komagaeibacter hansenii, Komagaelbacter medellinensis, Komagaeibacter europaeus, Komagaeibacter maltaceti, Komagaeibacter pomaceti, Komagaeibacter oboediens, or Komagaeibacter saccharivoans;
   v) a bacterial cell selected from the group comprising or consisting of:
      a) a strain of Komagaelbacter rhaeticus selected from the group comprising or consisting of: Komagaelbacter rhaeticus IGEM, Komagaelbacter rhaeticus AF1; Komagaelbacter rhaeticus LMG22126; or
      b) Gluconacetobacter xylinus CGMCC 2995; or
   vi) a bacterial cell that is a Komagaeibacter rhaeticus iGEM cell.
32. An optogenetic expression system for use in bacteria of the genus Komagataeibacter, comprising:
(a) A first nucleic acid comprising a first nucleotide sequence that encodes a first polypeptide, wherein the first polypeptide comprises:
   i) a first domain that comprises a first portion of a heterologous split-polymerase or a split-transcription factor; and
   ii) a second domain that comprises a first light-inducible dimerization domain;
(b) A second nucleic acid comprising a second nucleotide sequence that encodes a second polypeptide, wherein the second polypeptide comprises:
   i) a first domain that comprises a second portion of a heterologous split-polymerase or a split-transcription factor; and
   ii) a second domain that comprises a second light-inducible dimerization domain;
and
(c) A third nucleic acid comprising a third nucleic acid sequence that encodes a target protein or RNA to be expressed operably linked to a target promoter;
and wherein the first light-inducible dimerization domain and the second light-inducible dimerization domain are capable of dimerising with one another upon exposure to light of a dimerization wavelength to form a functional heterologous polymerase or a functional transcription factor capable of transcribing or initiating transcription from the target promoter,
and wherein the target promoter is recognised by the functional heterologous polymerase or functional transcription factor so as to drive transcription of the third nucleic acid sequence that encodes a target protein or RNA.
33. The optogenetic expression system according to paragraph 32 wherein the target promoter is a heterologous promoter.
34. The optogenetic expression system according to paragraph 32 or 33 wherein:
   the first nucleic acid, the second nucleic acid and the third nucleic acid are all part of the same nucleic acid molecule; or
   the first nucleic acid, the second nucleic acid and the third nucleic acid are different nucleic acid molecules.
35. The optogenetic expression system according to paragraphs 32 or 33, wherein the first nucleic acid and the second nucleic acid are part of the same nucleic acid molecule, and the third nucleic acid is part of a different nucleic acid molecule;
36. The optogenetic expression system according to any of paragraphs 32-35 wherein the heterologous split-polymerase is a split-T7 polymerase.
37. The optogenetic expression system according to any of paragraphs 35-36 wherein the functional heterologous polymerase is a T7 polymerase.
38. The method according to any of paragraphs 32-35 wherein the split-transcription factor is a split-LuxR.
39. The method according to paragraph 38 wherein the target promoter comprises a LuxR binding site.
40. The optogenetic expression system according to any of paragraphs 32-39 wherein:
a) the first nucleic acid comprises a first promoter operably linked so as to drive expression of the first polypeptide; and/or
b) the second nucleic acid comprises a second promoter operably linked so as to drive expression of the second polypeptide.
41. The optogenetic expression system according to any of paragraphs 32-37 or 40, wherein:
   the first portion of the heterologous split-polymerase comprises or consists of a sequence of at least 70%, at least 75%, at least 80%, at least 85%, at least 90%, at least 95%, at least 96%, at least 97%, at least 98%, or at least 99% Identical to SEQ ID NO: 1 and/or SEQ ID NO: 27, or 100% Identical to SEQ ID NO: 1 and/or SEQ ID NO: 27; and/or
   the second portion of the heterologous split-polymerase comprises or consists of a sequence of at least 70%, at least 75%, at least 80%, at least 85%, at least 90%, at least 95%, at least 96%, at least 97%, at least 98%, or at least 99% Identical to SEQ ID NO: 2, SEQ ID NO: 33, and/or SEQ ID NO: 35 or 100% Identical to SEQ ID NO: 2, SEQ ID NO: 33, and/or SEQ ID NO: 35.
42. The optogenetic expression system according to any of paragraphs 32-37 or 40-41, wherein:
   the first portion of the heterologous split-polymerase is encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 3 and/or SEQ ID NO: 26 or 100% identical to SEQ ID NO: 3 and/or SEQ ID NO: 26; and/or
   the second portion of the heterologous split-polymerase is encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 4, SEQ ID NO: 32, and/or SEQ ID NO: 34 or 100% identical to SEQ ID NO: 4, SEQ ID NO: 32, and/or SEQ ID NO: 34.
43. The optogenetic expression system according to any of paragraphs 32-37 or 40-42, wherein the first portion of the heterologous split-polymerase and the second portion of the heterologous split-polymerase comprises or consists of a pair of sequences selected from:
   SEQ ID NO: 1 and SEQ ID NO: 2;
   SEQ ID NO: 27 and SEQ ID NO: 33; or
   SEQ ID NO: 1 and SEQ ID NO: 35;
   or sequences that are at least 70%, at least 75%, at least 80%, at least 85%, at least 90%, at least 95%, at least 96%, at least 97%, at least 98%, or at least 99% Identical thereto, or 100% Identical thereto.
44. The optogenetic expression system according to any of paragraphs 32-37 or 40-43, wherein the first portion of the heterologous split-polymerase and the second portion of the heterologous split-polymerase are encoded by a DNA sequence that comprises or consists of a pair of sequences selected from:
   SEQ ID NO: 3 and SEQ ID NO: 4;
   SEQ ID NO: 26 and SEQ ID NO: 32; or
   SEQ ID NO: 3 and SEQ ID NO: 34;
   or sequences that are at least 70%, at least 75%, at least 80%, at least 85%, at least 90%, at least 95%, at least 96%, at least 97%, at least 98%, or at least 99% identical thereto, or 100% Identical thereto.
45. The optogenetic expression system according to any of paragraphs 32-42 wherein:
   the first light-inducible dimerization domain is a LOV dimerization domain and the second light-inducible dimerisation domain is a LOV dimerisation domain;
   the first light-inducible dimerization domain is an nMag dimerization domain and the second light-Inducible dimerisation domain is a pMag dimerisation domain;
   the first light-inducible dimerization domain is a pMag dimerization domain and the second light-inducible dimerisation domain is an nMag dimerisation domain;
   the first light-inducible dimerization domain is a VVD dimerization domain and the second light-inducible dimerization domain is a VVD dimerization domain;
   the first light-Inducible dimerization domain is a LOVtrap dimerization domain and the second light-inducible dimerisation domain is an LOVtrap dimerisation domain;
   the first light-inducible dimerization domain is a VfAU1-LOV dimerization domain and the second light-inducible dimerisation domain is a VfAU1-LOV dimerisation domain;
   the first light-inducible dimerization domain is a NgPA1-LOV dimerization domain and the second light-Inducible dimerisation domain is a NgPA1-LOV dimerisation domain;
   the first light-inducible dimerization domain is a OdPA1-LOV dimerization domain and the second light-inducible dimerisation domain is a OdPA1-LOV dimerisation domain;
   the first light-inducible dimerization domain is a AsLOV2 dimerization domain and the second light-inducible dimerisation domain is an PDZ dimerisation domain;
   the first light-inducible dimerization domain is a PDZ dimerization domain and the second light-inducible dimerisation domain is a AsLOV2 dimerisation domain;
   the first light-inducible dimerization domain is a AtCry2 dimerization domain and the second light-inducible dimerisation domain is a AtCry2 dimerisation domain;
   the first light-inducible dimerization domain is a PhyB dimerization domain and the second light-inducible dimerisation domain is a PIF dimerisation domain;
   the first light-inducible dimerization domain is a PIF dimerization domain and the second light-inducible dimerisation domain is a PhyB dimerisation domain;
   the first light-inducible dimerization domain is a Cph1 dimerization domain and the second light-inducible dimerisation domain is a Cph1 dimerisation domain; or
   the first light-inducible dimerization domain is a CBD dimerization domain and the second light-inducible dimerisation domain is a CBD dimerisation domain.
46. The optogenetic expression system according to paragraph 43 wherein:
the nMag dimerization domain comprises or consists of a sequence that is at least 70%, at least 75%, at least 80%, at least 85%, at least 90%, at least 95%, at least 96%, at least 97%, at least 98%, or at least 99% identical to SEQ ID NO: 5 or 100% Identical to SEQ ID NO: 5; and/or
the pMag dimerization domain comprises or consists of a sequence that is at least 70%, at least 75%, at least 80%, at least 85%, at least 90%, at least 95%, at least 96%, at least 97%, at least 98%, or at least 99% Identical to SEQ ID NO: 6 or 100% Identical to SEQ ID NO: 6, SEQ ID NO: 31, and/or SEQ ID NO: 45 or 100% Identical to SEQ ID NO: 6, SEQ ID NO: 31, and/or SEQ ID NO: 45.
47. The optogenetic expression system according to any of paragraphs 43 or 44 wherein:
   the nMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% identical to SEQ ID NO: 7 or 100% identical to SEQ ID NO: 7 and/or
   the pMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% identical to SEQ ID NO: 8, SEQ ID NO: 30, and/or SEQ ID NO: 44 or 100% Identical to SEQ ID NO: 8, SEQ ID NO: 30, and/or SEQ ID NO: 44 or 100% Identical to SEQ ID NO: 8, SEQ ID NO: 30, and/or SEQ ID NO: 44 or 100% Identical to SEQ ID NO: 8, SEQ ID NO: 30, and/or SEQ ID NO: 44.
48. The optogenetic expression system according to any of the preceding paragraphs wherein the first nucleic acid sequence comprises or consists of a nucleotide sequence having at least 70%, at least 75%, at least 80%, at least 85%, at least 90%, at least 95%, at least 96%, at least 97%, at least 98%, or at least 99% Identical to any of SEQ ID NO: 9, 10, 11, 12, 28, 36, 38, 40, or 42.
49. The optogenetic expression system according to any of the preceding paragraphs wherein the first dimerization domain and the second dimerization domain are substantially incapable of dimerization in the absence of light of the dimerization wavelength.
50. The optogenetic expression system according to any of the preceding paragraphs, wherein the dimerization wavelength is about 400 nm to 500 nm; optionally a wavelength of between 400 nm and 500 nm, optionally 450 nm.
51. The optogenetic expression system according to any of the preceding paragraphs wherein where:
the first light-Inducible dimerization domain is an nMag dimerization domain and the second light-inducible dimerisation domain is a pMag dimerisation domain; or
the first light-inducible dimerization domain is a pMag dimerization domain and the second light-inducible dimerisation domain is an nMag dimerisation domain;
then the light of a dimerization wavelength has a wavelength of about 400 nm to 500 nm; optionally a wavelength of between 400 nm and 500 nm, optionally 450 nm.
52. The optogenetic expression system according to any of the preceding paragraphs wherein the third nucleotide sequence is capable of being transcribed into mRNA, optionally wherein the mRNA is capable of being translated into a polypeptide.
53. The optogenetic expression system according to any of the preceding paragraphs wherein the third nucleotide sequence encodes a polypeptide.
54. The optogenetic expression system according to any of the preceding paragraphs wherein the third nucleotide sequence encodes a polypeptide that:
a) is involved in the biosynthesis of a pigment visible to the naked eye or in the biosynthesis of a molecule that emits light; or
b) encodes a protein that emits light or is a pigment.
55. The optogenetic expression system according to any of the preceding paragraphs wherein the polypeptide that is involved in the biosynthesis of a pigment visible to the naked eye is an enzyme necessary for the formation of melanin, optionally wherein the melanin is selected from the group comprising eumelanin, pheomelanin, neuromelanin and allomelanin, optionally wherein the polypeptide is a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
56. The optogenetic expression system according to paragraphs 54 or 55 wherein the polypeptide is involved in the biosynthesis of a pigment visible to the naked eye, and wherein expression of the polypeptide that is involved in the biosynthesis of a pigment visible to the naked eye results in the formation of the pigment,
   optionally results in the formation of melanin, optionally wherein the melanin is selected from the group comprising eumelanin, pheomelanin, neuromelanin and allomelanin, optionally wherein the polypeptide is a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
57. The optogenetic expression system according to any of the preceding paragraphs wherein the protein that emits light emits light emits light of a second wavelength when exposed to light of a first wavelength.
58. The optogenetic expression system according to any of the preceding paragraphs wherein the protein that emits light is a fluorescent protein, optionally selected from the group comprising or consisting of:
mCherry, GFP, mScarlet, mRFP, cjBlue, gfasPurple, eforRed, spisPink
59. The optogenetic expression system according to any of the preceding paragraphs wherein where the melanin is eumelanin, the third nucleotide encodes tyrosinase, optionally a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
60. The optogenetic expression system according to any of the preceding paragraphs wherein the third nucleotide sequence comprises the coding sequence for Tyr1, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
61. The optogenetic expression system according to any of the preceding paragraphs wherein the first promoter and/or the second promoter are inducible promoters.
62. The optogenetic expression system according to any of the preceding paragraphs wherein the first promoter and/or the second promoter are constitutive promoters.
63. The optogenetic expression system according to any of the preceding paragraphs further comprising a fourth nucleic acid sequence that encodes a heterologous protein required for inducible expression from the first and/or second promoter, optionally wherein the heterologous protein is a transcriptional regulator.
64. The optogenetic expression system according to any of the preceding paragraphs wherein the first and/or second promoter is selected from the group comprising or consisting of:
P_{BAD} [SEQ ID NO: 18];
pLux [SEQ ID NO: 19];
pTet [SEQ ID NO: 20]; or
pLac [SEQ ID NO: 21];
optionally wherein the promoter comprises or consists of a sequence with at least 80%, 82%, 84%, 86%, 88%, 90%, 92%, 94%, 96%, 98%, 99% or 100% sequence identity to any of the above sequences.
65. The optogenetic expression system according to any of the preceding paragraphs wherein the first and second promoter are both inducible promoters, optionally selected from the following inducible systems:
the pBAD promoter [SEQ ID NO: 18] induced by arabinose in the presence of the transcriptional regulator araC [SEQ ID NO: 22];
the pLux promoter [SEQ ID NO: 23] induced by Acyl Homoserine Lactone (AHL) in the presence of the transcriptional regulator LuxR [SEQ ID NO: 23];
the pTet promoter [SEQ ID NO: 20] induced by Anhydrotetracycline (ATc) in the presence of the transcriptional regulator TetR [SEQ ID NO: 24]; or
the pLac promoter [SEQ ID NO: 21] induced by IPTG in the presence of the transcriptional regulator LacI [SEQ ID NO: 25];
optionally wherein:
the first promoter is pBAD and the second promoter is pBAD;
the first promoter is pLUX and the second promoter is pLUX;
the first promoter is pTet and the second promoter is pTet;
the first promoter is pLac and the second promoter is pLac;
optionally wherein the promoter comprises or consists of a sequence with at least 80%, 82%, 84%, 86%, 88%, 90%, 92%, 94%, 96%, 98%, 99% or 100% sequence identity to [SEQ ID NO: 18], [SEQ ID NO: 19], [SEQ ID NO: 20], [SEQ ID NO: 21], and
optionally wherein the transcriptional regulator comprises or consists of a sequence with at least 80%, 82%, 84%, 86%, 88%, 90%, 92%, 94%, 96%, 98%, 99% or 100% sequence identity to [SEQ ID NO: 22], [SEQ ID NO: 23], [SEQ ID NO: 24], [SEQ ID NO: 25].
66. The optogenetic expression system according to any of the preceding paragraphs wherein the first and second promoters are both inducible promoters that are induced by the same inducer.
67. The optogenetic expression system according to paragraph 64 wherein the first and second promoters are both induced by arabinose.
68. The optogenetic expression system according to any of the preceding paragraphs wherein the first and second promoters comprise the P_{BAD} promoter sequence, optionally comprise or consist of [SEQ ID NO: 18] or a sequence that has at least 80%, 82%, 84%, 86%, 88%, 90%, 92%, 94%, 96%, 98%, 99% or 100% sequence identity to [SEQ ID NO: 18].
69. The optogenetic expression system according to paragraphs 32-68 wherein the fourth nucleic acid sequence encodes a transcriptional regulator selected from the group comprising or consisting of:
araC [SEQ ID NO: 22] or a sequence with at least 80%, 82%, 84%, 86%, 88%, 90%, 92%, 94%, 96%, 98%, 99% or 100% sequence identity to [SEQ ID NO: 22]; LuxR [SEQ ID NO: 23] or a sequence with at least 80%, 82%, 84%, 86%, 88%, 90%, 92%, 94%, 96%, 98%, 99% or 100% sequence identity to [SEQ ID NO: 23]; TetR [SEQ ID NO: 24] or a sequence with at least 80%, 82%, 84%, 86%, 88%, 90%, 92%, 94%, 96%, 98%, 99% or 100% sequence identity to [SEQ ID NO: 24]; LacI [SEQ ID NO: 25] or a sequence with at least 80%, 82%, 84%, 86%, 88%, 90%, 92%, 94%, 96%, 98%, 99% or 100% sequence identity to [SEQ ID NO: 25].
70. The optogenetic expression system according to any of paragraphs 32-69 wherein the fourth nucleic acid sequence encodes a heterologous protein required for inducible expression from the first and/or second promoter, optionally wherein the heterologous protein is a transcriptional regulator.
71. The optogenetic expression system according to paragraph 68, wherein the transcriptional regulator is araC.
72. The optogenetic expression system according to any of the preceding paragraphs wherein any of the first nucleotide sequence, the second nucleotide sequence, the third nucleotide sequence, and/or the fourth nucleotide sequence are operably linked to an enhancer sequence, a terminator sequence, a repressor sequence, an operator sequence and/or a sigma factor binding site.
73. The optogenetic expression system according to any of the preceding paragraphs wherein:
   the first nucleic acid, the second nucleic acid, the third nucleic acid and optional fourth nucleic acid are all part of the same nucleic acid molecule; or
   the first nucleic acid, the second nucleic acid, the third nucleic acid an optional fourth nucleic acid are different nucleic acid molecules.
74. The optogenetic expression system accord to any of the preceding paragraphs wherein the first nucleic acid and the second nucleic acid are part of the same nucleic acid molecule, and the third nucleic acid is part of a different nucleic acid molecule.
75. The optogenetic expression system according to any of the preceding paragraphs wherein any one or more of the first nucleic acid, the second nucleic acid, the third nucleic acid an optional fourth nucleic acid are circular nucleic acids, optionally are selected from the group consisting of a plasmid, a bacterial artificial chromosome, a phagemid, a cosmid, a yeast artificial chromosome, a human artificial chromosome, a viral vector, or any combination thereof.
76. The optogenetic expression system according to any of the preceding paragraphs wherein all of the first nucleic acid, the second nucleic acid, the third nucleic acid and optional fourth nucleic acid are present in the same circular nucleic acid, optionally present in the same circular nucleic acid selected from the group consisting of a plasmid, a bacterial artificial chromosome, a phagemid, a cosmid, a yeast artificial chromosome, a human artificial chromosome, a viral vector.
77. The optogenetic expression system according to paragraph 75 or 76 wherein the circular nucleic acid, optionally a plasmid, further comprises an origin of replication.
78. The optogenetic expression system according any of the proceeding paragraphs wherein any one or more of the nucleic acids further comprises a selectable marker.
79. A cell comprising the optogenetic expression system of any of paragraphs 32-78.
80. The cell of paragraph 79, wherein the cell is capable of producing bacterial cellulose.
81. The cell according to either of paragraphs 79 or 80, wherein the cell is selected from the group comprising or consisting of: a bacterial cell, an archaeal cell, or a eukaryotic cell.
82. The cell according to any of paragraphs 79-81, wherein the cell is a bacterial cell, optionally a bacterial cell that expresses all of bcsA, bcsD, bscC and bscD.
83. The cell of paragraph 82, wherein the bacterial cell belongs to a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes.
84. The cell of any of paragraphs 79-83, wherein the bacterial cell is selected from the group comprising or consisting of: Komagaeibacter rhaeticus; Komagaeibacter xylinus, Komagaelbacter hansenii, Komagaeibacter medellinensis, Komagaeibacter europaeus, Komagaeibacter maltaceti, Komagaelbacter pomaceti, Komagaelbacter oboediens, or Komagaelbacter saccharivoans.
84. The cell of any of paragraphs 79-84, wherein the bacterial cell is:
a) a strain of Komagaeibacter rhaeticus selected from the group comprising or consisting of: Komagaeibacter rhaeticus iGEM. Komagaeibacter rhaeticus AF1; Komagaeibacter rhaeticus LMG22126; or
b) Gluconacetobacter xylinus CGMCC 2995.
86. The cell of paragraph 85, wherein the bacterial cell is a Komagaeibacter rhaeticus iGEM cell.
87. The cell of any of paragraphs 79-86, wherein the first and/or second and/or third and/or fourth nucleic acid of the optogenetic expression system according to any of paragraphs 32-78 Is Integrated into the genome of the cell, optionally wherein:
   i) the first and optionally second and optionally fourth nucleic acids of the optogenetic expression system are integrated into the genome of the cell; or
   ii) all nucleic acids of the optogenetic expression system are integrated into the genome of the cell.
88. The cell of any of paragraphs 79-86, wherein the first and/or second and/or third and/or fourth nucleic acid of the optogenetic expression system according to any of paragraphs 24-67 are maintained episomally within the cell, optionally wherein:
   i) where the first and optionally second and optionally fourth nucleic acids of the optogenetic expression system are integrated into the genome of the cell according to paragraph 87 i), the third nucleic acid of the optogenetic system is maintained episomally within the cell; or
   ii) all nucleic acids of the optogenetic expression system are maintained episomally within the cell.
89. A method of producing spatially pigmented bacterial cellulose, comprising the steps of:
(a) providing a culture of the cells according to any of paragraphs 79-88 wherein the third nucleotide sequence encodes a polypeptide that:
   i) is involved in the biosynthesis of a pigment visible to the naked eye or in the biosynthesis of a molecule that emits light; or
   ii) encodes a protein that emits light or is a pigment;
(b) maintaining the culture of (a) under conditions that allow the production of a cellulose pellicle by the microorganism; and
(c) exposing a spatially defined region or regions of the cellulose pellicle to light of the dimerization wavelength so as to allow expression of the third polypeptide.
90. The method according to paragraph 89 wherein the cellulose pellicle in (b) is allowed to develop to the final desired area and/or thickness prior to exposing the defined region or regions to light in step (c).
91. The method according to any of paragraphs 89 or 90 wherein once the pellicle in (b) has developed to the final desired area and/or thickness it is harvested prior to exposing the spatially defined region or regions to light in step (c).
92. The method according to paragraph 89 wherein the spatially defined regions of the cellulose pellicle are exposed to the light during step (b).
93. The method according to paragraph 89-92 wherein the volume of the culture is kept constant during exposure to the light.
94. The method according to any one or more of paragraphs 89-93 wherein the region or regions of the cellulose pellicle that are not to be exposed to light are protected using a mask.
95. The method according to paragraph 94 wherein the mask is placed as close as possible to the surface of the pellicle, optionally wherein the mask contacts the surface of the pellicle.
96. The method according to paragraphs 94 or 95 wherein the mask is entirely opaque.
97. The method according to paragraphs 94 or 95 wherein the mask comprises at least some regions that are semi-transparent so as to allow a reduced intensity of light to reach the pellicle in at least some areas.
98. The method according to any of paragraphs 89-97 the preceding paragraphs wherein the strength of expression of the third polypeptide that:
   i) is involved in the biosynthesis of a pigment visible to the naked eye or in the biosynthesis of a molecule that emits light; or
   ii) encodes a protein that emits light or is a pigment;
is modulated by varying:
   a) the intensity of light that the pellicle or culture is exposed to; and/or
   b) the duration of exposure to light.
99. The method according to any of paragraphs 89-98 wherein where:
   i) the first nucleic acid comprises a first promoter operably linked so as to drive expression of the first polypeptide; and
   ii) the second nucleic acid comprises a second promoter operably linked so as to drive expression of the second polypeptide,
and wherein when the first and second promoter are inducible promoters,
then the strength of expression of the third polypeptide that:
   i) is involved in the biosynthesis of a pigment visible to the naked eye or in the biosynthesis of a molecule that emits light; or
   ii) encodes a protein that emits light or is a pigment;
is modulated by varying:
   a) the intensity of light that the pellicle or culture is exposed to;
   b) the duration of exposure to light; and/or
   c) the concentration of inducing agent that the pellicle or culture is exposed to;
optionally where the first promoter and second promoter are arabinose inducible promoters, the cell is engineered to also express AraC and the inducing agent is arabinose.
100. The method according to any of paragraphs 89-99 wherein the third polypeptide that:
(a) is involved in the biosynthesis of a pigment visible to the naked eye or in the biosynthesis of a molecule that emits light; or
b) encodes a protein that emits light or is a pigment;
is an enzyme necessary for the formation of melanin, optionally wherein the melanin is selected from the group comprising eumelanin, pheomelanin, neuromelanin and allomelanin.
101. The method according to paragraph 100 wherein the third polypeptide is tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
102. A method for spatially restricted gene expression in bacterial cellulose wherein the method comprises:
(a) providing a culture of the cells according to any of paragraphs 78-88;
(b) maintaining the culture of (a) under conditions that allow the production of a cellulose pellicle by the cells; and
(c) exposing a defined region or regions of the cellulose pellicle to light of the dimerization wavelength so as to allow dimerization of the first and second light-inducible dimerization domain and formation of the functional heterologous polymerase and transcription of the third nucleic acid sequence that encodes a target protein or RNA to be expressed.
103. The method according to paragraph 102 wherein the cellulose pellicle in (b) is allowed to develop to the final desired area and/or thickness prior to exposing the defined region or regions to light in step (c).
104. The method according to any of paragraphs 102 or 103 wherein once the pellicle in (b) has developed to the final desired area and/or thickness it is harvested prior to the defined region or regions to light in step (c).
105. The method according to paragraph 102 wherein the spatially defined regions of the cellulose pellicle are exposed to the light during step (b).
106. The method according to any of paragraphs 102-105 wherein the volume of the culture is kept constant during exposure to the light.
107. The method according to any of paragraphs 102-106 wherein the region or regions of the cellulose pellicle that are not to be exposed to light are protected using a mask.
108. The method according to paragraph 107 wherein the mask is placed as close as possible to the surface of the pellicle, optionally wherein the mask contacts the surface of the pellicle.
109. The method according to paragraphs 107 or 108 wherein the mask is entirely opaque.
110. The method according to paragraphs 107 or 108 wherein the mask comprises at least some region or regions that are semi-transparent so as to allow a reduced intensity of light to reach the pellicle in at least some areas.
111. The method according to any of paragraphs 102-110 wherein the strength of expression from the third nucleic acid sequence that encodes a target protein or RNA to be expressed is modulated by varying:
a) the intensity of light that the pellicle or culture is exposed to; and/or
b) the duration of exposure to light.
112. The method according to any of paragraphs 102-111 wherein where:
i) the first nucleic acid comprises a first promoter operably linked so as to drive expression of the first polypeptide; and
ii) the second nucleic acid comprises a second promoter operably linked so as to drive expression of the second polypeptide,
and wherein the first and second promoter are inducible promoters,
then the strength of expression from the third nucleic acid sequence that encodes a target protein or RNA to be expressed is modulated by varying:
   a) the intensity of light that the pellicle or culture is exposed to;
   b) the duration of exposure to light; and/or
   c) the concentration of inducing agent that the pellicle or culture is exposed to;
optionally where the first promoter and second promoter are arabinose inducible promoters, the inducing agent is arabinose.
113. The method according to any of paragraphs 102-112 wherein the third nucleic acid sequence encodes an enzyme necessary for the formation of melanin,
optionally wherein the melanin is selected from the group comprising eumelanin, pheomelanin, neuromelanin and allomelanin.
113. The method according to paragraph 112 wherein the third nucleic acid encodes
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.
115. A method for producing a bacterial cellulose pellicle that can be spatially pigmented upon exposure to light, wherein the method comprises:
(a) providing a culture of the cells according to any of paragraphs 66-75;
(b) maintaining the culture of (a) under conditions that allow the production of a cellulose pellicle by the microorganism until a pellicle of the appropriate area and/or thickness has been obtained; and
(c) harvesting the pellicle; and
wherein the pellicle has not been exposed to light of the dimerization wavelength.
116. The method according to any of paragraphs 89-115 wherein the cells are cultured in step (b) a culture medium that:
i) is Hestrin and Schramm (HS) medium;
ii) is supplemented with glucose, optionally at 2% (w/v); and/or
iii) is buffered to a pH of 5.8.
117. A method for spatially pigmenting bacterial cellulose wherein the method comprises:
a) providing a bacterial cellulose pellicle that has been produced by a culture of cells according to any of paragraphs 66-75, optionally by the method according to paragraph 102 or 103; and
b) exposing spatially restricted areas of the pellicle to light of the dimerization wavelength.
118. The method according to any of paragraphs 89-114 and 116 wherein the pellicle is exposed to light of the dimerization wavelength for:
   at least 1 hour, 2, 3, 4, 5, 6, 7, 8, 9, 11, 12, 18, 24, 36, 48, 60, 72, 84, 96 hours, 5 days, 6 days, 7 days, 2 weeks, 1 month or more; and/or
   less than 1 month, less than 2 weeks, 7 days, 6 days, 5 days, 96 hours, 84, 72, 60, 48, 36, 24, 18, 12, 11, 9, 8, 7, 6, 5, 4, 3, 2, 1 hours.
119. The method according to any of paragraphs 89-114 and 116 wherein following exposure of the pellicle to light, the method further comprises exposing the pellicle to a pigment development solution, optionally wherein the pigment development solution:
   comprises metal ions with an oxidation state of 2+;
   is at a pH of between 6 and 8.5; and
   comprises L-tyrosine and/or L-cysteine and/or L-cystine.
120. The method according to any of paragraphs 89-114 or 116-119 wherein following exposure to light or following exposure to a pigment development solution, the pellicle is sterilised, optionally sterilised by:
   i) autoclaving;
   ii) heating; and/or
   iii) desiccation, optionally with 70% ethanol.
121. A spatially pigmented bacterial pellicle as produced according to any method of any of the preceding paragraphs, optionally wherein the pigment is melanin.
122. A pigmented bacterial pellicle as produced according to any method of any of the preceding paragraphs, optionally wherein the pigment is melanin.
123. A bacterial pellicle suitable for light-induced spatially restricted pigmentation wherein the bacterial pellicle has been produced according to the method of paragraph 115 and wherein the pellicle has not been exposed to light of the dimerization wavelength.
124. The spatially pigmented bacterial pellicle of paragraph 121, or the bacterial pellicle of paragraph 121, wherein the bacterial pellicle comprises the cell according to any of paragraphs 79-88.
125. The pigmented bacterial pellicle of paragraph 122, wherein the bacterial pellicle comprises the cell according to paragraph 31 or according to any of paragraphs 79-88.
126. A textile comprising a bacterial pellicle according to paragraphs 122 or 125.
127. A textile comprising a bacterial pellicle according to paragraph 121, or 123, or 124.
128. An apparatus for exposing spatially defined regions of a bacterial pellicle to light comprising:
i) a light source to illuminate a surface of the bacterial pellicle;
ii) a light diffuser
iii) a mask, optionally a transparency; and
iv) a lens;
wherein the distance between the light source and the surface of the bacterial pellicle can be adjusted
optionally wherein the lens autofocuses on the surface of the pellicle
wherein the light source is a low voltage and/or a low wattage light source, optionally a low voltage and/or a low wattage LED flood lamp with a wattage less than 100 W, 80 W, 60 W, 50 W, 40 W, 30 W, 20 W, or 10 W or less; and/or 10 W or more, 20 W, 30 W, 40 W, 50 W, 60 W, 60 W, 100 W or more.
129. Use of the apparatus according to paragraph 128 in the spatially restricted pigmentation of a bacterial pellicle produced according to any of the preceding paragraphs.
129. Use of a digital projector in the spatially restricted pigmentation of a bacterial pellicle produced according to any of the preceding paragraphs.
130. A pigment development solution, wherein the solution:
a) comprises metal ions with an oxidation state of 2+; optionally comprises:
   i) a) Cu²⁺, Zn²⁺, Be²⁺, Mg²⁺, Ca²⁺, Cr²⁺, Mn²⁺, Co²⁺ or Ni²⁺;
      b) Cu²⁺, Zn²⁺; and/or
      c) Cu²⁺; and/or
   ii) CuSO₄, optionally comprises:
      at least 2 μM CuSO₄, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuSO₄; and/or
      between 2 μM CUSO₄ and 20 μM CUSO₄; and/or
      less than 20 μM, optionally less than 17.5 μM, 15 μM, 12.5 μM, 10 μM, 7.5 μM, 5 μM, 4 μM, 3 μM, or less than 2 μM; and/or
      between 20 μM and 160 μM CuSO₄; and/or
      less than 160 μM, optionally less than 140 μM, 120 μM, 100 μM, 80 μM, 60 μM, 40 μM, or less than 20 μM CuSO₄; and/or
   iii) CuCl₂, optionally comprises:
      at least 2 μM CuCl₂, optionally at least 3 μM, 4 μM, 5 μM, 7.5 μM, 10 μM, 12.5 μM, 15 μM, 17.5 μM or at least 20 μM40 μM, 60 μM, 80 μM, 100 μM, 120 μM, 140 μM, or at least 160 μM CuCl₂; and/or
      between 2 μM CuCl₂ and 20 μM CuCl₂; and/or
      less than 20 μM, optionally less than 17.5 μM, 15 μM, 12.5 μM, 10 μM, 7.5 μM, 5 μM, 4 μM, 3 μM, or less than 2 μM; and/or
      between 20 μM and 160 μM CuCl₂; and/or
      less than 160 μM, optionally less than 140 μM, 120 μM, 100 μM, 80 μM, 60 μM, 40 μM, or less than 20 μM CuCl₂;
and
b) is at a pH of:
   between 6 and 8.5, optionally between 6.25 and 8.25, 6.5 and 8, 6.25 and 7.75, 6.5 and 7.5, 6.75 and 7.25; and/or
   at least 6, optionally at least 6.25, 6.5, 6.75, 7, 7.25, 7.4, 7.5, 7.75, 8, 8.25 or at least 8.5; and/or
   7.4;
And
c) comprises L-tyrosine and/or L-cysteine and/or L-cystine, optionally comprises:
   i) at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L; between 0.1 g/L and 2 g/L tyrosine; and/or
      less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine;
      and/or
   ii) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
      between 10 g/L and 300 g/L cysteine; and/or
      less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
      and/or
   iii) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L; between 0.1 g/L and 2 g/L L-cystine; and/or
      less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine,
optionally wherein the development solution comprises:
a) PBS at pH 7.4;
b) 10 μM CuSO₄ or 20 μM CuSO₄; and 0.5 g/L L-tyrosine, 1 g/L L-tyrosine, 1 g/L cysteine, and/or 0.4 g/L cystine.
131. A kit comprising:
a) a bacterial pellicle as described in any of the above paragraphs; and
b) a development solution according to any of the preceding paragraphs.
132. A kit comprising:
a) a bacterial pellicle as described in any of the above paragraphs that comprises an optogenetic expression system according to any of the preceding paragraphs;
b) a development solution according to any of the preceding paragraphs; and optionally
c) a light source wherein the wavelength of the light results in dimerization of the first and second dimerization domains, optionally wherein the light source comprises the apparatus of paragraph 128.
133. A light-responsive bacterial cellulose material.
135. The light-responsive bacterial cellulose material of 133, wherein the material is produced by the method according to any of paragraph 89-118.
136. A product, method, or kit as described substantially herein.

The invention will now be exemplified by the following non-limiting examples.

### Example 1—K. rhaeticus Cellulose Secretion

The structure of bacterial cellulose pellicles produced by K. rhaeticus have already been imaged with SEM and TEM. However, neither of these methods are able to capture the active secretion of cellulose from growing cells, therefore, we used fluorescence microscopy to observe the active secretion of cellulose from living K. rhaeticus cells. K. rhaeticus was grown in the ONIX microfluidic system. The device contains multiple trap heights, which hold bacterial cells at the height that matches their diameter, maintaining them within a single plane. An HS media flow rate of ˜2.5 μL/hr was used to provide nutrients to K. rhaeticus. To improve the visibility of any secreted polysaccharides, we used the dye Fluorescent Brightener 28 (FB 28). The dye FB 28 has an affinity for glucose-based polysaccharides such as cellulose and chitin and is typically used for the staining of fungal and plant cell walls. The ability of FB 28 to bind to other glucose-based polysaccharides, not just cellulose, may also make visible other secreted polysaccharides. Indeed, most Komagataeibacter also have the genes required to produce and secrete acetan.

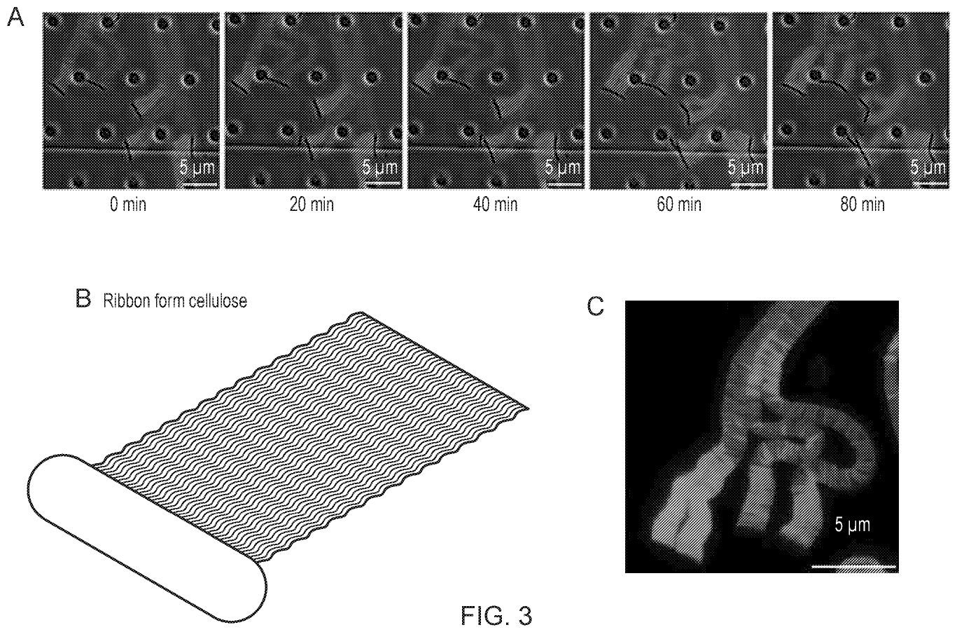

We successfully observed the secretion of two separate polysaccharide morphologies from K. rhaeticus (FIG. 3). A ribbon like polysaccharide morphology was observed being secreted from the longitudinal axis of K. rhaeticus cells (FIG. 3a). These ribbons bare a resemblance to cellulose bands observed secreted from K. xylinus with TEM (Hiral et al 2002 Cellulose 9: 105-113). The ribbon morphology was maintained across dividing cells, and overtime, the ribbons traced the lineage of cell division (FIG. 3c). In the microfluidic device, a constant flow of HS media moves from the bottom of the trap to the top. The ribbons do appear to be orientated with the flow of the media, which may explain why many of the ribbons appear to secrete from the same side across the cells observed. The other morphology observed was a thin and rope-like polysaccharide. Interestingly this morphology was much harder to observe with the FB28 dye and was more easily observed under phase contrast bright field (FIG. 3/e/f). In the previous referenced TEM study, the cellulose bands were actually observed folding up into rope like cellulose chains, similar in scale to those observed here. These polysaccharide ropes look similar to the cellulose fibres seen under the SEM of bacterial cellulose pellicles.

### Example 2—Pigmented Bacterial Cellulose

Bacterial cellulose presents a great case study in which we can explore the possibilities of autonomous pigment production in a living material. The question, however, is which pigments should we seek to produce to explore this potential. Looking to biology for inspiration, one of the most abundant, stable and chemically interesting pigments is melanin. The term melanin refers to a group of pigmented organic compounds that are produced in a broad variety of living organisms. The complexity and diversity of melanin compounds, and their aversion to structural characterisation by current chemical tools means our ability to classify and study melanin is still poor. Nonetheless, melanin compounds can be broken down into four groups: eumelanin, pheomelanin, neuromelanin and allomelanin. Eumelanin, pheomelanin and neuromelanin are all produced in animals, with the former two responsible for the diverse colouring of human hair, eyes and skin. Allomelanin refers to the melanin compounds typically produced from many non-animal sources, such as those produced by plants, fungi and bacteria. It should however be mentioned that many bacteria can and do produce eumelanin and pheomelanin through chemical pathways similar to that seen in mammalian systems (Nosanchuk and Casadevall 2006 Antimicrob Agents Chemother 50: 3519-3528). Of these four varieties of melanin, eumelanin has garnered the most intrigue and study, and may be a fitting compound to produce in an ELM.

The dark-brown/black pigment eumelanin is a heterogenous macromolecule composed of two components, 5,6-dihydroxyindole (DHI) and 5,6-dihydroxyindole-2-carboxylic acid (DHICA) (Meredith and Sarna 2006 Cell Res 19: 572-594). It is highly stable, with direct evidence of eumelanin discovered within the fossilised ink sacks of cephlapods. It is capable of the broad-band absorption of both UV and visible light, giving eumelanin its UV-protective abilities and dark black colour. These protective abilities even extend to protection from ionising radiation. One of the more eclectic features of eumelanin is its ability to act as an electrical conductor when wet. While a physical model that explains the conductivity (and photoconductivity) of eumelanin has not been settled upon, eumelanin has become a candidate material in the construction of organic and biocompatible electronics. Beyond the creation of a self-pigmenting material, producing eumelanin within bacterial cellulose may yield a composite material with advanced material properties.

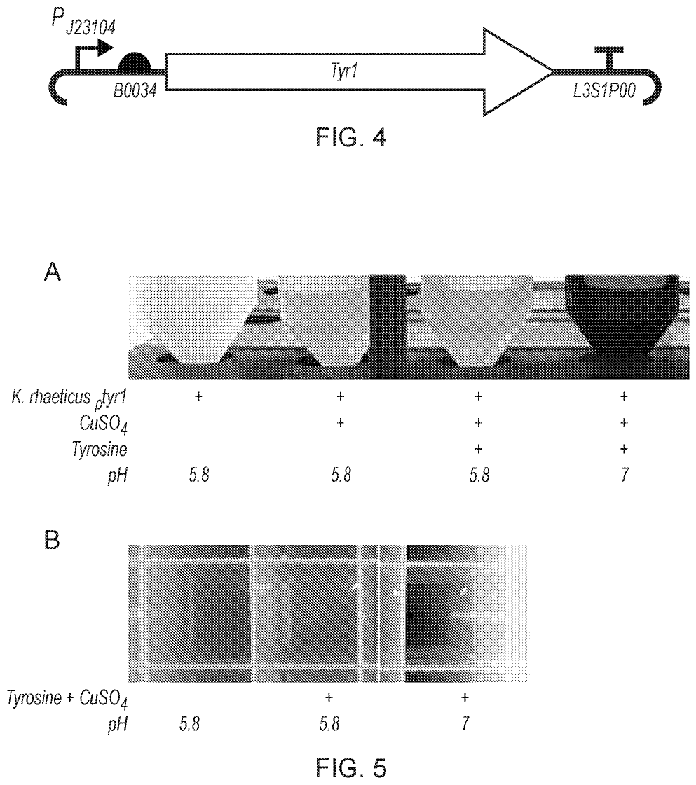

The biochemical pathway for eumelanin begins with the amino acid L-tyrosine which undergoes the enzyme dependant oxidation of tyrosine into dopaquinone. The enzyme involved here is known as a tyrosinase, a copper containing monophenol monoxidase, and is the only enzyme that is essential for the conversion of L-tyrosine to eumelanin. The dopaquinone molecule then undergoes the intramolecular addition of amine group to form cyclodopa, which then rapidly oxidises to form dopachrome. Due to the inherent reactivity of dopaquinone and cyclodopa, these reactions proceed without catalysis (FIG. 5.2). Dopachrome, further decomposes to form DHI and DHICA which then further oxidise together to form macromolecular eumelanin (Ito 2003 Pigment Cell Res 16: 230-236). As this pathway shows, if we wish to produce eumelanin in bacterial cellulose, then we must first express the tyrosinase enzyme within K. rhaeticus. To facilitate this process choosing a tyrosinase of bacterial origin is most likely to work.

The challenge of producing eumelanin in K. rhaeticus can be broken down into three steps. First, to build a plasmid construct, containing a suitable tyrosinase, that can be transformed into K. rhaeticus. Second, to determine the culturing conditions that would allow for a liquid K. rhaeticus culture to produce eumelanin. Third, using knowledge gained from those liquid cultures, establish a process for the production of melanated bacterial cellulose pellicles.

Recombinant eumelanin work has mainly been conducted with tyrosinases from two sources, namely the MelA protein (including the mutant form, MutMelA) from R. etli and the Tyr1 protein from B. megaterium. With both Komagataeibacter and Rhizobium belonging to Alphaproteobacteria, K. rhaeticus is much closer to R. etli than to the Gram-positive B. megaterium. Yet despite this fact, we decided to focus our efforts on the expressing Tyr1 in K. rhaeticus due to its smaller size, broader range of recombinant test cases and its proven use in fusion proteins and synthetic biological circuits.

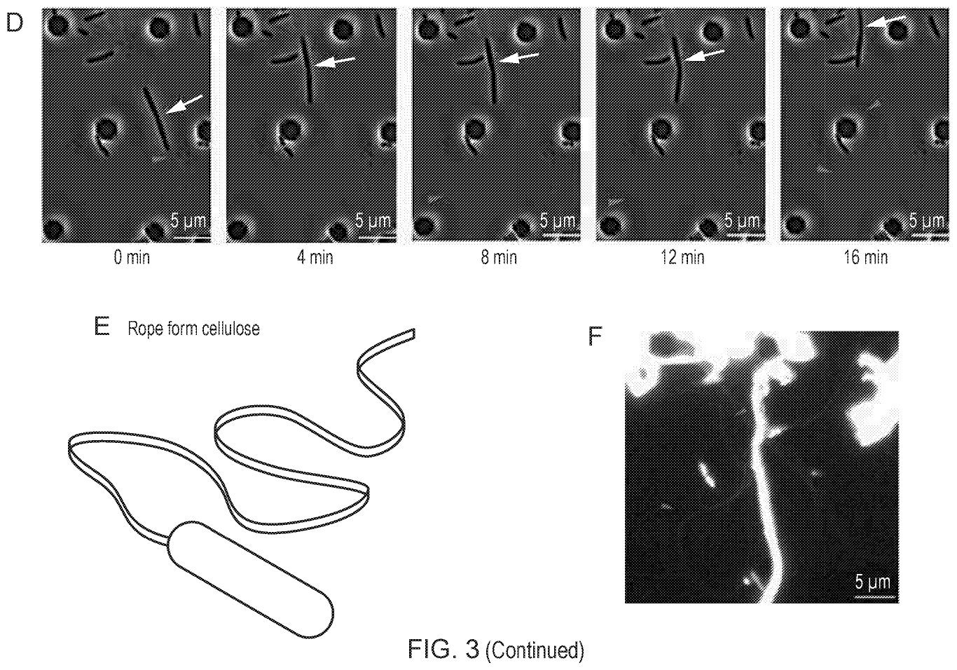

First, the DNA sequence for the tyr1 gene (adapted for use in the KTK (Goosens et al. bioRxiv 2021.06.09.44769)) was synthesised by Twist Biosciences. Upon arrival, the tyr1 gene fragment was cloned into the KTK e1.3 entry vector. Using destination vector d1.2, the tyr1 gene was placed upstream of the high-expression constitutive Anderson promotor J23104 and RBS B0034, which has been frequently used in K. rhaeticus for the expression of fluorescent proteins (FIG. 4). We decided to use constitutive over inducible expression for consistency in tyrosinase expression in pellicles. This may be especially important in the production of the large pellicles, where the slow diffusion of chemical inducers can lead to uneven protein expression. The resulting plasmid, ptyr1, was then transformed into K. rhaeticus.

While the creation of a Tyr1 expression plasmid for K. rhaeticus was simple, an incompatibility between the optimum tyrosinase conditions and the culturing conditions of K. rhaeticus was more of a challenge. Tyrosinase dependant eumelanin synthesis is thought to be dependent upon certain conditions: access to tyrosine, copper (II) ions, oxygen, a temperature of 30° C. and pH 7. Of these conditions, the antimicrobial properties of copper and the difficulty of achieving a neutral pH with an acetic acid bacterium stand out. We therefore grew K. rhaeticus ptyr1 shaking with cellulase in a selection of conditions to identify those that allowed for growth and eumelanin synthesis.

Previous studies had shown that 10 μM of copper sulphate was suitable for eumelanin synthesis with Tyr1 (Shuster and Fishman 2009 J Mol Microbiol Biotechnol 17: 188-200). We therefore added 10 μM of copper sulphate to HS media and inoculated this media with K. rhaeticus ptyr1. After two days, the culture had become turbid, and K. rhaeticus growth was unaffected by the copper content of the media (FIG. 5a). HS media is buffered with a citric acid-phosphate buffer at a pH of 5.8, which is well below the optimal pH for eumelanin synthesis, we therefore grew K. rhaeticus ptyr1 In HS media, set at both pH 5.8 and pH 7, both media also contained 10 μM CuSO₄ and 0.5 g/L L-tyrosine to make eumelanin production possible. After 2 days of growth, the two cultures were checked. The HS media at pH 5.8 showed no colour change, whilst the HS media set to pH 7 had turned a deep black colour, clearly indicating the successful production of eumelanin (FIG. 5a).

Following the identification of conditions that allowed for eumelanin production in K. rhaeticus, we then set out to grow melanated K. rhaeticus ptyr1 pellicles. We inoculated K. rhaeticus ptyr1 into HS media set to pH 7 with 10 μM CuSO₄, 0.5 g/L L-tyrosine, and allowed the culture to stand for 7 days. After Day 7 the culture was checked however no pellicle had formed and a small volume of dark sediment had formed at the bottom of the well (FIG. 5b). To ensure that K. rhaeticus ptyr1 could still form pellicles, K. rhaeticus ptyr1 was also inoculated into HS media at pH 5.8 both with and without copper and tyrosine. In these cases, both media were able to produce pellicles (FIG. 5b). This suggested that either a media pH of 7 makes it impossible for K. rhaeticus ptyr1 to form pellicles and/or that eumelanin production from these conditions led to a toxicity or fitness defect that prevents pellicle formation.

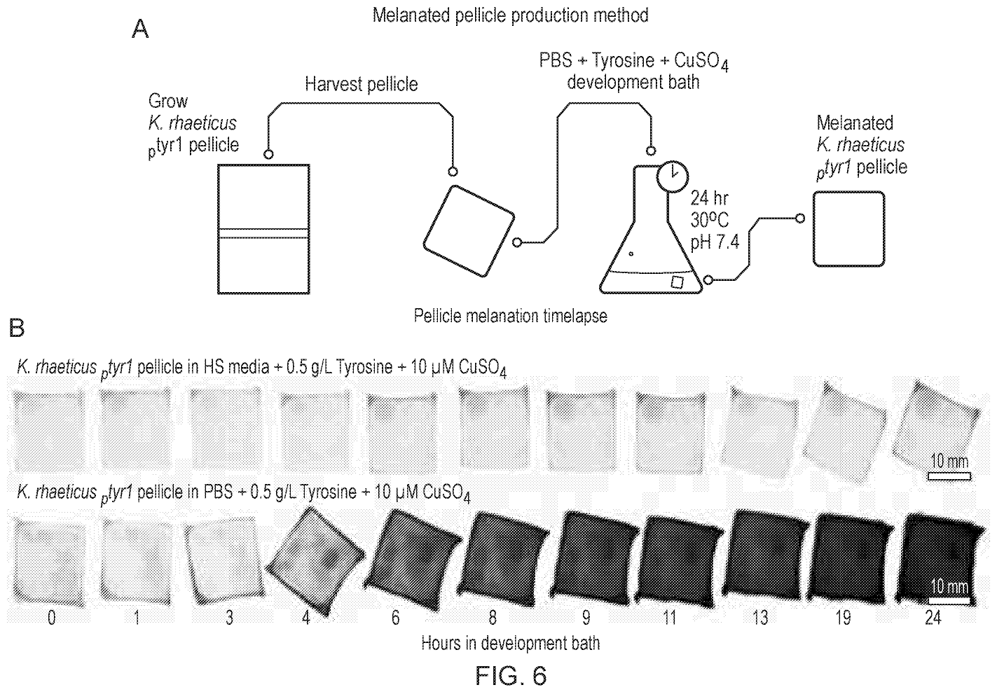

To avoid the pH incompatibilities, we decided to develop a two-stage approach to producing melanated BC pellicles (FIG. 6a).

In this approach, we first cultured K. rhaeticus ptyr1 in standard HS media at pH 5.8 to allow a pellicle to develop. Once the pellicle reached the desired thickness, the pellicle was harvested and washed in sterile water to remove any remaining media from the outside of the pellicle. The washed pellicle was then placed in a development bath of PBS at pH 7.4, with 10 μM CUSO₄, 0.5 g/L L-tyrosine and incubated at 30° C. with shaking to increase aeration. With this approach, we were successful in producing melanated bacterial cellulose pellicles. We found that the pellicle reached a maximum perceivable darkness after 24 hours in the PBS development bath (FIG. 6b). We also investigated whether altering L-tyrosine concentrations would allow us to control the degree of darkening. We found that including 0.5 g/L L-tyrosine in both the growth media and development bath led to the highest level of darkening (FIG. 6c). We hypothesise that including copper and L-tyrosine in the growth media removes the need for them to diffuse into the pellicle during the development bath, leading to greater and more even melanin production throughout the pellicle. Indeed, pellicles grown with 10 μM CuSO₄ and 0.5 g/L L-tyrosine then placed into pure PBS without copper and L-tyrosine still underwent darkening, indicating a suitable amount of tyrosine and copper had diffused into the pellicle.

### Example 3—Sterilisation

For applications outside of a laboratory context, it was important that we be able to sterilise the melanated pellicles without destroying the eumelanin within them. A series of melanated pellicles were produced and subjected to a range of conditions for pellicle sterilisation (FIG. 6d). The highly oxidising methods of sterilisation—household bleach and hydrogen peroxide—had a strong bleaching effect on the eumelanin, meaning neither method is suitable for melanated pellicle sterilisation. While alkali, 0.1 M NaOH and acidic, 0.1 M HCl, methods of sterilisation showed little loss in pellicle colour, eumelanin could be observed leaching out of the melanated pellicles. This led to a fading of colour around the rim of the pellicle, suggesting strongly acid and alkali solutions are also not suitable methods of melanated pellicle sterilisation. Methods that depended upon heat, autoclaving, and desiccation, 70% ethanol, proved to be the most suitable methods of sterilisation, with the sterilised melanated pellicles showing no discernible differences in colour before and after sterilisation. Additionally, the ability of the melanated pellicles to remain unfaded after autoclaving suggested the eumelanin is highly colour-fast to water-based washing conditions.

### Example 4—Effect of Eumelanin on K. rhaeticus

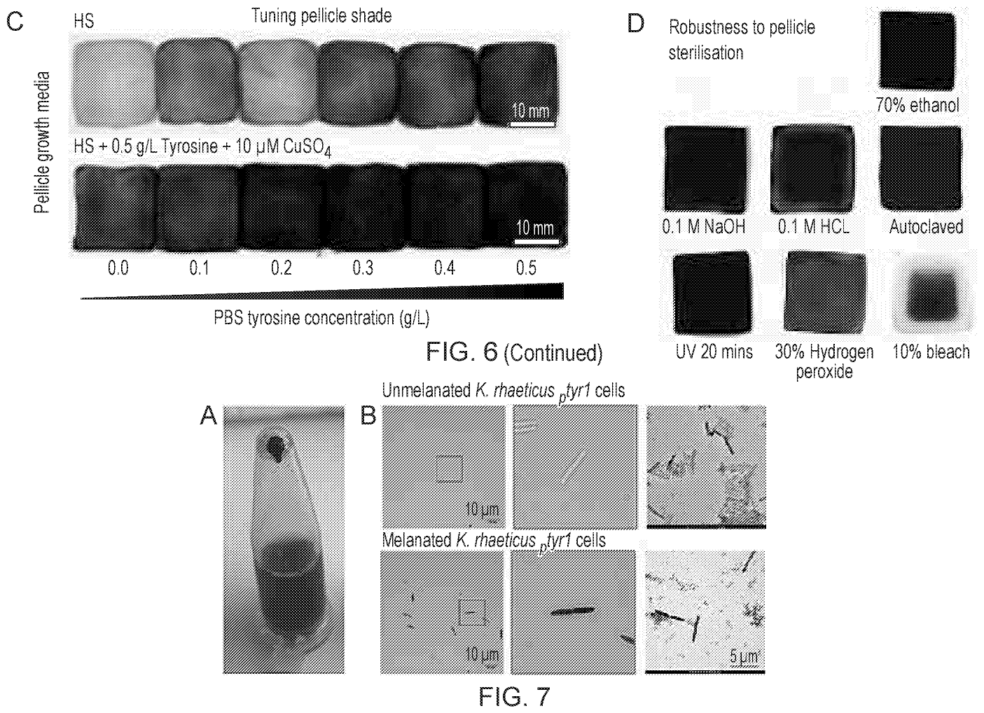

After successfully producing eumelanin from K. rhaeticus, we then sought to understand where the eumelanin accumulated and how this accumulation affected the physiology of K. rhaeticus. The location of eumelanin synthesis, either intra or extracellularly, is important in understanding how or If the eumelanin macromolecules interact with the extracellular bacterial cellulose. Using centrifugation on a liquid melanated K. rhaeticus ptyr1 culture to pellet the cells produced a black pellet and dark brown supernatant, revealing that some portion of the eumelanin produced does exist outside of the cell (FIG. 7a). Considering that active secretion of Tyr1 by K. rhaeticus Is highly unlikely without the necessary secretion signals we hypothesised that some amount of eumelanin or, indeed, Tyr1 protein is released into the media through cell lysis.

We then used microscopy to study how eumelanin production had affected the physical properties of the K. rhaeticus ptyr1 cell. We used K. rhaeticus ptyr1 that had not been through the melanin development process (unmelanated) as a control. Melanated and unmelanated K. rhaeticus ptyr1 cells were placed on an agarose pad and observed under a microscope (FIG. 7b). The melanated cells were considerably darker than the unmelanated cells, suggesting that eumelanin accumulates either within or on K. rhaeticus ptyr1 cells. Finally, we used TEM to look for changes in cellular morphology resulting from eumelanin production (FIG. 7b). No significant differences in cellular structure were observed.

### Example 5—Investigating the Effect of Eumelanin Production on Bacterial Cellulose

The exact role that eumelanin and other eumelanin-like macromolecules play in giving some biomaterials their materials properties is not fully understood. Eumelanin-like compounds—the “like” here really reflecting the difficulty in classifying melanin compounds—are found frequently in biology with the carbohydrate biomaterial chitin, with the squid beak being a typical example. Considering the chemical similarity of cellulose and chitin, we were therefore curious to know how eumelanin production may have impacted the material properties of bacterial cellulose.

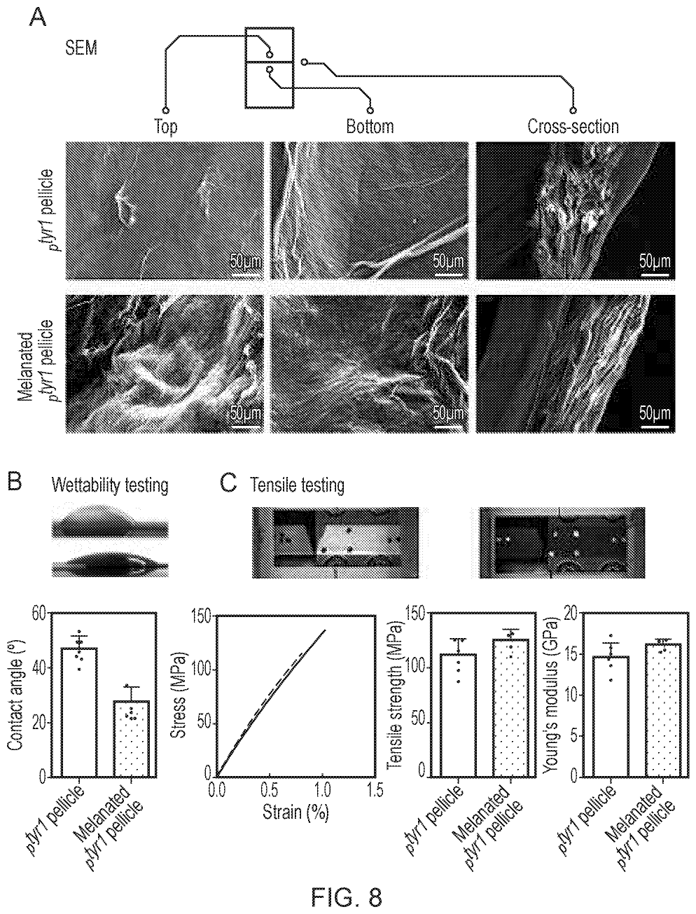

To Investigate how eumelanin production may have altered the surface of a bacterial cellulose pellicle we used scanning electron microscopy (SEM). We compared the top and bottom surfaces, as well as the cross-sections, of melanated and unmelanated K. rhaeticus ptyr1 pellicles (FIG. 8a). In preparing the two pellicles for SEM, measures were taken to ensure the unmelanated pellicle received as similar treatment as possible to the melanated pellicle. The unmelanated pellicle was prepared by placing it into a low pH buffer, which prevented eumelanin synthesis, and incubated in identical conditions to the melanated pellicle in the development bath. The SEM images do not show large differences in the surface architecture between the melanated and unmelanated pellicles, however one can notice the melanated pellicles do seem to have a rougher and fuzzier surface.

To further study the surface material properties of melanated cellulose, we conducted wettability testing using the static sessile drop method (FIG. 8b). The melanated pellicle showed increased surface wettability, with the melanated pellicle possessing an average contact angle of 28° compared to 47° for the unmelanated pellicle, suggesting the melanated cellulose was significantly more wettable than unmelanated cellulose. This increase in wettability may be a reflection of the rougher surface, identified by SEM, of the melanated pellicle. One of the most attractive features of bacterial cellulose is its high tensile strength, which is similar to Kevlar. Tensile testing was carried out on both melanated and unmelanated pellicles. The results showed that the melanated and unmelanated pellicles had similar material properties, implying that while eumelanin production did not enhance the material properties of bacterial cellulose, the material properties were not hindered either.

### Example 6—an Optogenetic System for K. rhaeticus

When choosing a system for producing patterned bacterial cellulose, we were conscious of the restrictions and unknowns that conducting synthetic biology in K. rhaeticus present. We therefore prioritised choosing a simple optogenetic system that did not require membrane bound parts, additional enzymes for chromophore synthesis or double plasmid transformation. One system that stood out for its simplicity and orthogonality was the blue light-inducible T7 RNA-Polymerase system (T7-Opto) (Baumschlager et al 2017 ACS Synth Biol 6: 2157-2167). The T7-Opto system is dependent on a pair of blue light sensitive “magnet” proteins, nMag and pMag. In the presence of blue light, these nMag and pMag proteins dimerise. This feature of magnet proteins is converted into a light sensitive method of gene expression by splitting the T7 RNA polymerase into two separate parts, and then fusing N-terminal part with nMag protein and the C-terminal part with pMag protein. In the presence of blue light, these two fusion proteins dimerise, creating an active T7 RNA polymerase that can initiate transcription from the PT7 promoter. This system has many features that make it a good fit for working in non-model organisms such as K. rhaeticus. The nMag and pMag proteins use flavin as a chromophore, which is ubiquitous in bacterial cells, meaning the system does not require the additional engineering of chromophore synthesis to function. While the T7-Opto system originally used a two plasmid approach, with both the split T7 RNA polymerase on one plasmid, and target gene, under control of the PT7 promoter, on the other plasmid, this arrangement can easily be compressed on to one plasmid. The orthogonal transcription of the T7 RNA polymerase also removes the uncertainty of requiring non-native regulators having to interact with native sigma factors in K. rhaeticus to initiate transcription.

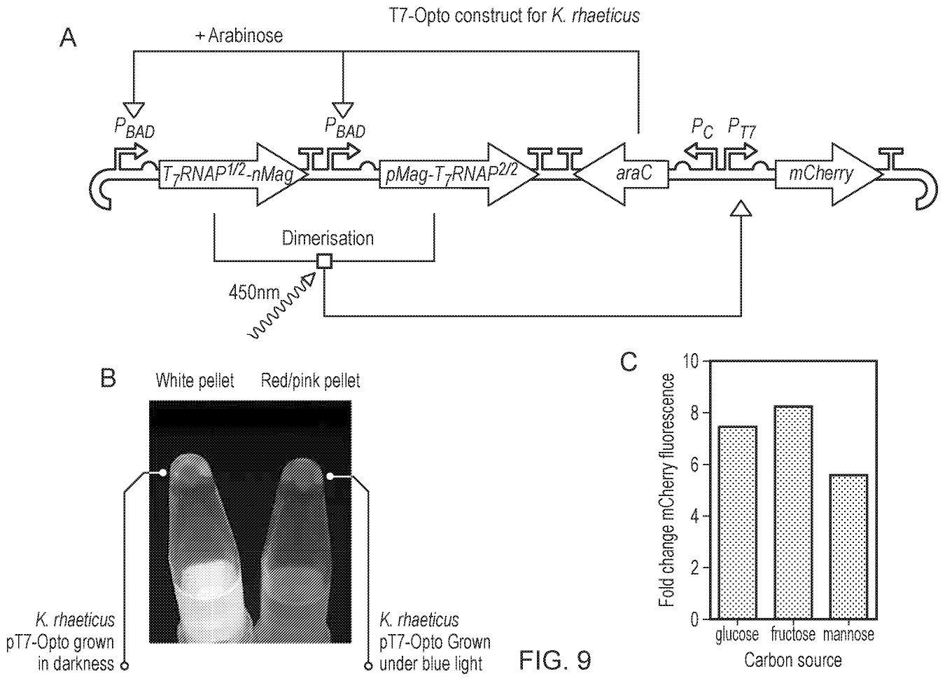

To test the functionality T7-Opto system in K. rhaeticus, we first had to adapt the build of the system. At the time of construction, a plasmid pair for the stable double transformation of K. rhaeticus had not been demonstrated. We therefore placed both the split T7 RNA polymerase and the PT7 controlled mCherry onto the same plasmid. The plasmid backbone used had a pBBR1 origin and chloramphenicol resistance and is commonly used as a K. rhaeticus vector. The original T7-Opto system made use of the PBAD promoter to control the expression of both halves of the split T7 RNA polymerase. We decided to continue to use the PBAD promoter in the K. rhaeticus adapted T7-Opto system. However, since the araC regulator gene is not found natively in K. rhaeticus, the araC gene, under constitutive expression, was also added to the final construct. The final T7-Opto system adapted for K. rhaeticus involved four genes. The araC gene constitutively produces AraC, which in the presence of arabinose, induces the expression of both halves of the split T7 RNA polymerase. In the presence of blue light, the nMag and pMag domains of the split T7 RNA polymerase dimerise, creating an active T7 RNA polymerase that then transcribes the mCherry gene, leading to an increase in red fluorescence (FIG. 9a).

To test whether this optogenetic system worked in K. rhaeticus, we grew K. rhaeticus pT7-Opto shaking with 2% cellulase and 0.1% arabinose in darkness and under blue light. To provide the blue light, a strip of blue LED lights was placed alongside the K. rhaeticus pT7-Opto cultures in a small light tight incubator. After 2 days of growth, the cell pellets from the two cultures were compared, and the K. rhaeticus pT7-Opto culture grown under blue light appeared visibly redder than the dark culture pellet, implying the pT7-Opto system was functioning in K. rhaeticus (FIG. 9b). The cultures were then compared using flow cytometry which found that the K. rhaeticus pT7-Opto culture exposed to blue light had a 7.5-fold increase in mCherry expression compared to the dark culture (FIG. 9c). In E. coli, the PBAD system is repressed in the presence of glucose—a process referred to a catabolite repression. Since K. rhaeticus is grown on HS media containing 2% glucose, we were curious to investigate whether the induction response of the T7-Opto system to light, which is regulated by the PBAD promoter, was significantly repressed in the presence of glucose compared to alternative 6-carbon sugars. K. rhaeticus pT7-Opto was grown shaking with 2% cellulase in both dark and blue light in HS media with 2% fructose and 2% mannose. Flow cytometry showed all carbon sources produced a similar fold change in mCherry fluorescence from growth under blue light (FIG. 3.1c). This indicated that HSglucose is a suitable media to use for work involving K. rhaeticus pT7-Opto.

With the T7-Opto system proven functional in K. rhaeticus, we begun to further characterise the behaviour of the system in order to facilitate the rational design of patterned bacterial cellulose. For this specific system, the rational design is dependent on understanding the impact on mCherry production from changing key parameters in the system, namely, the intensity of blue light and the concentration of the inducer arabinose. We therefore developed an experiment that could the show the change in mCherry accumulation over multiple intensities of blue light and monitor how this response to blue light changes with arabinose concentration.

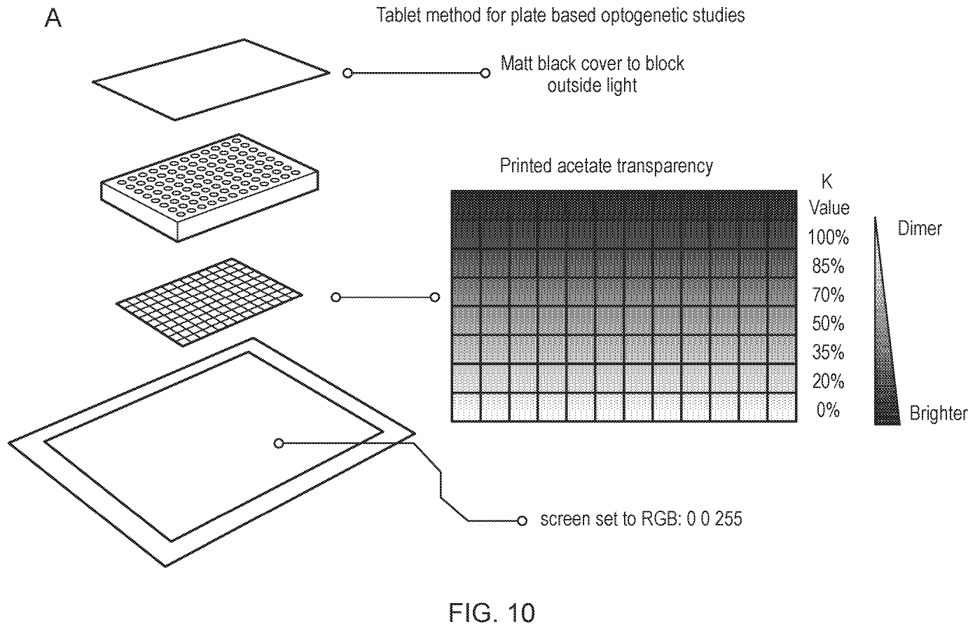

We took an initial approach that used an easily sourced tablet computer and laser printer to assemble a setup capable of exposing the individual wells of a 96 well plate to different intensities of light (FIG. 10a). To control the intensity of light reaching the wells, an 8×12 grid is printed onto an acetate transparency, with the shaded density of each cell controlling the amount of light that can pass through the transparency. The 8×12 grid transparency is placed onto the underside of a clear-bottomed black 96 well plate, which is then both placed atop a tablet screen illuminated to a specific colour. The full assembly could be placed into an incubator to conduct experiments at a specific temperature, and, if suitably adhered to the incubator platform, can also be shaken. With this tablet method we were able to monitor how the response to blue light of K. rhaeticus pT7-Opto changed with arabinose concentration.

The experiment revealed that the response of K. rhaeticus pT7-Opto to blue light intensity varied significantly with arabinose concentration (FIG. 10b). The impact of arabinose was tested to a high inducer concentration of 10% (w/v). This was chosen to encapsulate the finding of a previous study on arabinose induction in K. rhaeticus, that showed an optimum induction concentration of 2% (w/v) arabinose. Curiously, even without arabinose, mCherry accumulation followed a sigmoidal response to increasing blue light intensity. This showed that without induction, the leakiness of the PBAD promoter still produced enough split T7 RNA polymerase to dimerise and transcribe the mCherry gene. An arabinose concentration of 0.1% (w/v) also produced a sigmoidal response to blue light. Interestingly, the changes in blue light response from dark to bright, between no arabinose and 0.1% (w/v) arabinose were the same at 2.57-fold. At 0.1% arabinose, the induction response appears less digital than without arabinose. This was due to an increase in the sensitivity, as cells responded to lower intensities of blue light. At higher concentrations of arabinose, 1% (w/v) and 10% (w/v), the response to blue light appeared to diminish. The fold change between dark and light states was reduced, to 1.33-fold for 1% (w/v) arabinose and 1.04 fold for 10% (w/v) arabinose. Across all concentrations of arabinose, the mCherry accumulation in darkness is similar, suggesting the leakiness of the T7-Opto system is unaffected by the level of arabinose induction.

The fold changes between dark and blue light states do appear much reduced in this plate based set up compared to the tube based set up seen in the initial experiments. Potentially this reflects a less favourable growth environment for K. rhaeticus growing in a 96 well plate and would align with previous experiences when using plate readers to produce growth curves of K. rhaeticus, where poor cell growth and cellulose production hampered accurate readings of doubling time. However, it should be noted that without a standardised quantification of blue light intensity, it is difficult to make comparisons across experiments.

### Example 7—Spatial Patterning with K. rhaeticus pT7-Opto: Proof-of-Concept

With an understanding of how K. rhaeticus pT7-Opto responds to blue light, we then used K. rhaeticus pT7-Opto to produce a spatially patterned pellicle. The challenge of producing spatially patterned bacterial cellulose through optogenetics can be broken down into separate engineering and biological components. The engineering component is to find a suitable way of patterning blue light onto the surface of a growing bacterial cellulose pellicle. The biological component looks to understand how a K. rhaeticus pT7-Opto pellicle, responds to patterned blue light and how biological features of the T7-Opto system, such as leakiness and maximum output, shape the final expressed pattern. Fulfilling both of these components would establish a foundation for the rational design of patterned bacterial cellulose.

Starting with the challenge of patterning light onto a growing BC pellicle, we looked to photography for inspiration. In many optogenetic studies involving bacteria, the patterning of light onto a lawn of bacteria has been conducted by placing a mask close to the surface of the growing cells and illuminating the cells through this mask. Here, the photographic parallel is the “Contact print”, in which a film negative is placed directly onto a photosensitive surface and then exposed to light. While this approach is simple, it requires that the mask is placed as close as possible to the light sensitive surface, hence the “contact”, in order to get the highest sharpness. While such a technique is possible for the growth of bacteria on a solid surface like agar, for patterning bacterial cellulose, the mask would need to be precariously placed near the air/water interface and would probably be too tedious to work reliably for patterning light onto the surface of a pellicle.

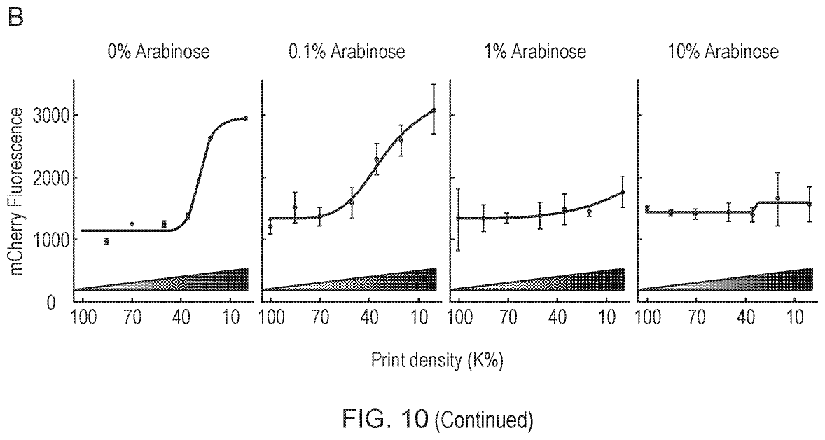

One of the more flexible tools for the patterning of light on to a surface, is the photographic enlarger. This device is used in the photo printing process, to project an image, typically from a film negative, onto a piece of photosensitive paper. The photographic enlarger can be broken down into a light source, beneath which sits the image to be projected in the form of a transparency. The light that passes through the transparency is then focused by a lens, which then projects the light on to the photosensitive surface beneath. The flexibility of the photographic enlarger comes from the ability to alter the distance between each of these components, changing the focus and scale of projection, and the aperture of the lens, which changes the amount of light projected onto the photosensitive surface. Given this flexibility, we wondered if a device, based on the photographic enlarger, could be developed with the specific purpose of producing spatially patterned bacterial cellulose through optogenetics. We therefore built a small, proof of concept, photographic enlarger (Enlarger V1) for patterning a growing pellicle (FIG. 11a).

The aim of the Enlarger V1 was to establish whether a photographic enlarger was a good basis for producing a patterned pellicle. The device would also allow for the understanding of which components should be adjusted in future iterations, with the aim to better answer the biological component of the patterning challenge. Looking at the individual components of the Enlarger V1 (FIG. 11b), the light source, a 10 W LED flood lamp, is at the top. Due to the long growth times of a bacterial cellulose pellicle, it was important that a low voltage LED light was used to reduce heat build-up. In front of the flood lamp, a translucent piece of paper is used to diffuse the LED light source, providing a smoother illumination of the mask placed underneath. For this mask, which took the form of the London Underground Roundel, the dark regions were made completely opaque, to block the maximum amount of blue light. This was done to offset the uncertainty around the sensitivity of K. rhaeticus pT7-Opto to blue light in the context of a growing pellicle. Underneath the image mask, sat the projection lens. To account for the minimum focal length of the lens, the image mask was spaced away from the lens. This lens then rested on an opaque piece of polystyrene, which suspended the back of the lens directly above the pellicle culture container. This piece of polystyrene also acted as a cover, to prevent contamination of the growing bacterial cellulose culture. Finally, at the bottom of the Enlarger V1 sat the pellicle culture container, which produces a 7 cm wide pellicle.

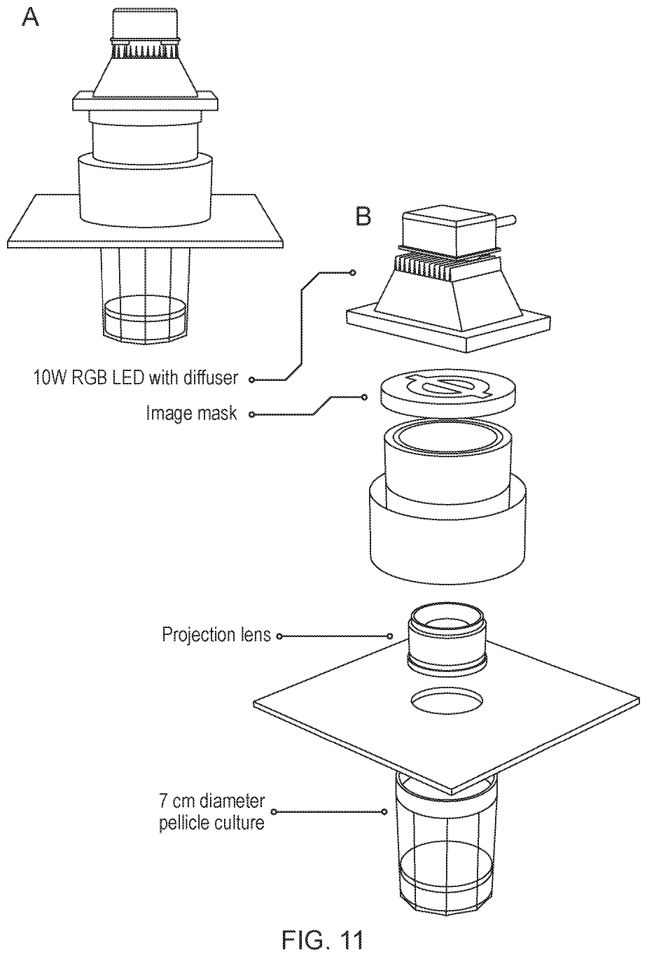

After assembly, the Enlarger V1 was then tested to see if it could produce a patterned pellicle. The Enlarger V1 was placed into a 30° C. light-tight incubator. The pellicle culture container was filed with 100 mL of HS media with 0.1% (w/v) arabinose and inoculated with K. rhaeticus pT7-Opto. The culture was left unilluminated, until a thin pellicle developed, at which point the LED lamp was turned on. After 3 days of blue light illuminated pellicle growth, the pellicle was harvested from the Enlarger V1. When illuminated with a green light, the final pellicle showed a red fluorescent roundel pattern indicating that the camera had worked. A high-resolution image of the pellicle was taken using a fluorescence scanner (FIG. 12a). Using this fluorescent scan, we looked at the intensity of the exposed and unexposed areas and found the average fluorescence in the exposed area was 1.8-fold that of the unexposed area (FIG. 12b).

The success of the Enlarger V1 proved that this method was a viable way to pattern gene expression in bacteria cellulose. We also gained insights on how to iterate the enlarger design. On the functional side, the current design made it impossible to alter the focus of the lens once pellicle growth had begun. The lens was therefore focused before growth to the expected liquid height. However, due to evaporation, this changed over time. As seen in the Enlarger V1 patterned pellicle, the roundel pattern was blurred around the edges. While this also likely due to the diffusion of light through the pellicle, the ability to fine tune the focus of the lens once the pellicle has formed would improve the sharpness of a patterned pellicle. The small size of the pellicle produced, limited the amount of detail that could be patterned onto the pellicle. With the next iteration of the enlarger, we planned to begin to understand the biological component of patterning with K. rhaeticus pT7-Opto.

### Example 8—Spatial Patterning with K. rhaeticus pT7-Opto: Rational Design of Patterned Cellulose

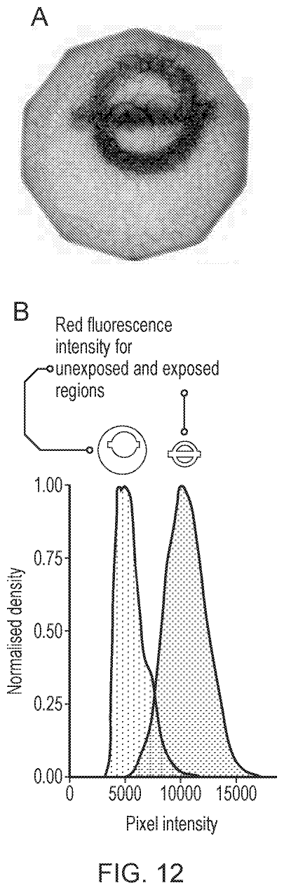

After the proof-of-concept test, we made a series of design changes to make a better enlarger (FIG. 13). Our main goal was to produce larger pellicles, which could hold more detailed patterns. In this iteration of the enlarger, we used a 14 cm diameter glass dish as a pellicle culture container. This increase in size however, required an increase in height of the entire enlarger. The LED lamp was scaled up to a 50 W blue LED flood lamp, which accounted for the increased distance from and width of the pellicle. To increase the flexibility of the whole design, we used a laboratory clamp to hold each of the components in place. The move to the clamp system made is much easier to change the distance between each of the components, facilitating focusing and scaling of the projected image. It also improved the accessibility of the lens during growth, allowing for the changing of focus without risk of disrupting and sinking the pellicle. We also moved from the stenciled mask method used in the first enlarger to a printed acetate transparency, an approach similar to that used in tablet method. This move allowed us to connect digital design with patterning of bacterial cellulose.

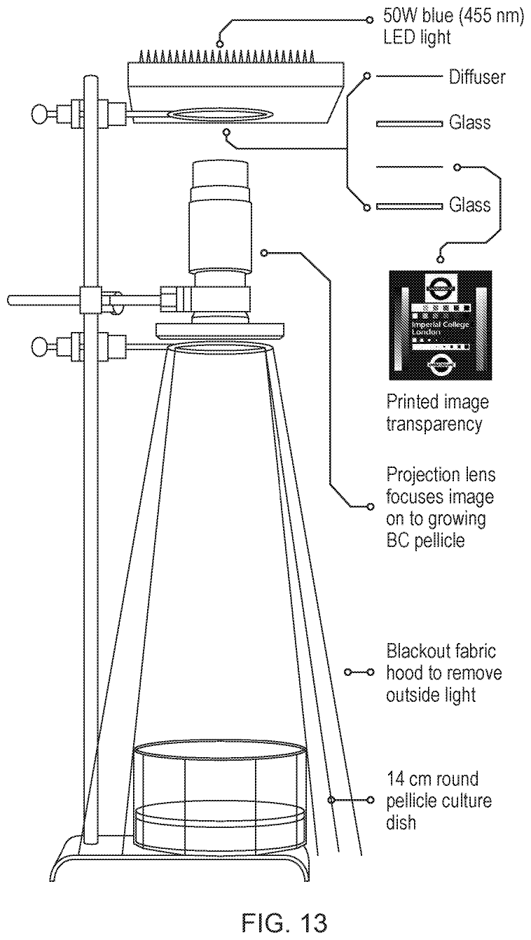

With this improved enlarger design in place, we then turned our attention to better understanding what was possible to pattern with K. rhaeticus pT7-Opto. As mentioned previously, our main aim was to facilitate the rational design of patterned bacterial cellulose. In the context of the Enlarger V2, this means that a transparency designed digitally for the enlarger would reliably produce the same pattern in the pellicle. To make this possible, we had to first gather information on the relationship between the projected image and the printed pellicle pattern. Again, inspiration for this can be found in photography and filmography. Faced with having to standardise image output across monitors and video equipment or characterise the photographic properties of film, the industry created test films, or test cards (FIG. 14a). These cards carried multiple tests on them for properties like resolution or colour rendering. We therefore carried this concept across with a test card designed for the rational design of patterned bacterial cellulose (FIG. 14b).

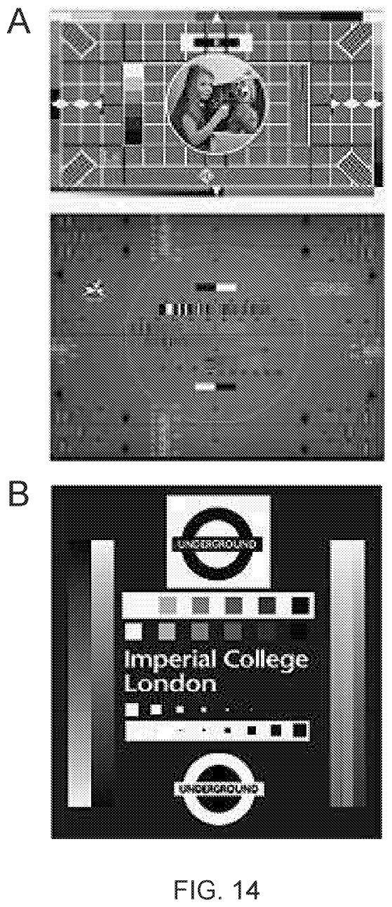

To conduct the optogenetic patterning with the Enlarger V2, we first inoculated 500 mL of HS media in the culture dish with K. rhaeticus pT7-Opto and added in 0.1% (w/v) arabinose. The lamp was turned on; the lens shuttered with a piece of black card and the full camera covered in a large box to keep the heat from the lamp inside and maintain conditions at ˜30° C. After 3 days of growth, a thin pellicle had formed in the culture dish. The lens was unshuttered and focus adjusted slightly until the sharpest possible image could be seen projected on to the pellicle. The pellicle was then exposed for 3 days, before being harvested and scanned with a fluorescence scanner. Whilst a fluorescent pattern could not be seen by eye, the fluorescence scan revealed the Enlarger v2 had indeed patterned mCherry expression, and the test card had been successfully printed on to the pellicle (FIG. 15a).

We then analysed the results of the test card print. Unfortunately, the test card was printed slightly offside, cutting of the left most gradient strip. Nonetheless, we were able to use the remaining gradient strip on the left side of the print, it appeared that the overall print was likely underexposed (FIG. 15b). The resolution of the print appeared to be high enough to render text as demonstrated by the imperial College London logo (FIG. 15c). The test card also looked to determine whether an image is better defined on a low expression or high expression background. Looking at the two roundel logos tested, it appeared, by eye, that an image on a low expression background shows better definition (FIG. 15d). The card also tested a range of possible shades, with the idea that one could use the results of this test to identify shades that provide suitable definition against both low and high exposure backgrounds, in future patterning attempts (FIG. 15e). The test identified four possible shades, that can be differentiated from a low exposure background. Whilst all 5 shades could be differentiated from a high exposure background, the four lightest printed shades could not be easily deciphered form each other. Finally, the card determined the minimum possible dot size (FIG. 15f). On both low expression and high expression backgrounds, the minimum possible printed mark was 0.8 mm in size. Smaller dots in the test card did not, by eye, leave any apparent marks in the final printed pellicle. With the successful printing of this test card, we had now set the foundation for the ration design of a future patterned pellicles using K. rhaeticus pT7-Opto.

### Example 9—Patterning Eumelanin Production within a Pellicle with Optogenetics

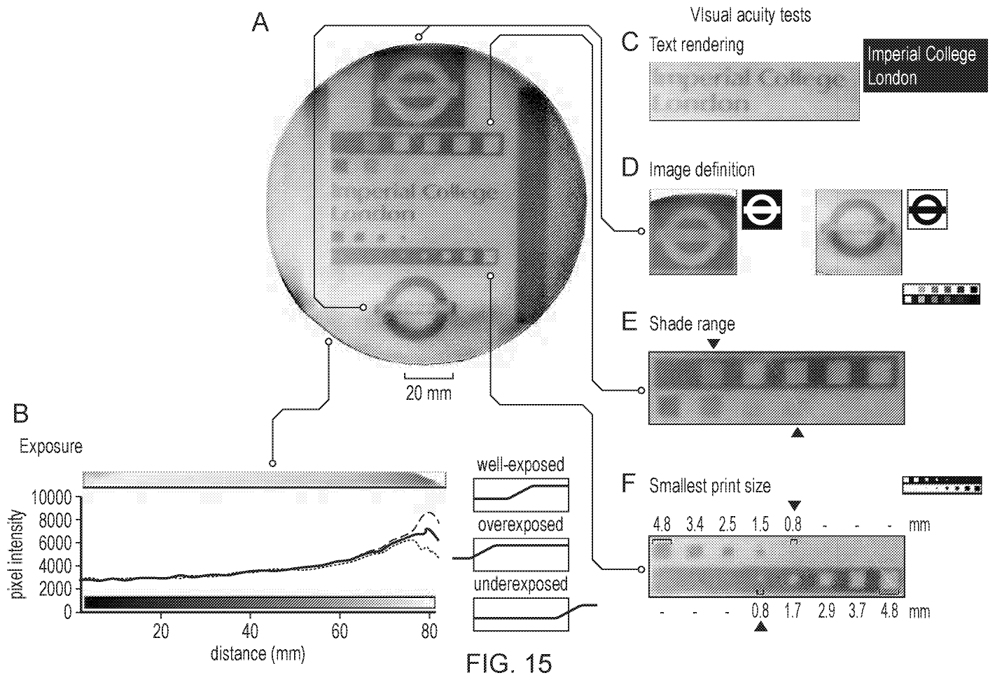

Advancing on the pT7-Opto system, we replaced the mCherry gene with tyr1, thereby creating a plasmid capable of producing the Tyr1 protein in response to blue light (FIG. 16a). Using the Enlarger V1, we grew and then exposed a K. rhaeticus PT7-Opto_tyr1 pellicle to a pattern of blue light (FIG. 16b). Upon harvesting the pellicle, we noticed a decrease in the opacity of pellicle region exposed to the light, indicating the region may have had a lower density of cells (FIG. 16c). Since this was not observed in the experiment with the T7-Opto system, we believe the decrease in cell density is unlikely to be due to effect of the blue light and may instead be due to an increase in cellular burden from the expression of the Tyr1 protein. Despite the apparent burden, we still attempted the eumelanin development process (FIG. 16d). Background expression of Tyr1 led to all parts of the pellicle darkening and the region exposed to light, most likely a result of the low cell density, appeared lighter than the unexposed regions of the pellicle. The background activity, or leakiness, of the T7opto system was not a significant issue in the patterning of mCherry.

However, its appearance in this attempt at patterning potentially speaks to the differences in the smaller amount of product required to produce an enzymatic signal, rather than the accumulative signal of fluorescent proteins.

Interestingly, the only regions that showed some signs of patterned eumelanin were the areas adjacent to the regions exposed to light. Since the light projected on to the pellicle likely diffused outwards upon hitting the pellicle surface, these adjacent regions were still exposed to a reduced intensity of light. Potentially, this reduced intensity of light led to increased Tyr1 production without increasing it so much to the point of significant cellular burden. This suggests that through optimisation of light intensity it may be possible to pattern eumelanin production with the current PT7-Opto_tyr1 system.

The experiment proved the system could express enough tyrosinase to lead to pellicle melanation.

However, burden led to reduced cell density in regions exposed to blue light.

Reducing the effect of burden through altering nutrient conditions, reducing split t7 polymerase production through arabinose concentration, and/or reducing blue light intensity is expected to increase cell density in light exposed regions.

This increased cell density may allow for a reduced development time, as the exposed region will contain more tyrosinase and develop before the leaky basal expression of tyrosinase in the unexposed regions can pigment the pellicle.

### Example 10—Melanated Bacterial Cellulose as a Textile

As we discussed in the introduction, some in the fashion industry are looking to biofabricated materials as the next generation of textiles. In many cases, it is the promise of more sustainable textiles that are driving such interest. Although, incidentally, “biofabricated” does not by definition equal “sustainable” and promises of sustainability from nascent technologies should always be scrutinised. For some however, biofabricated materials are seen as a way to open up the space for new methods of design, collaboration (with both multicellular eukaryotes and prokaryotes) and consumption of fashion. Even though synthetic textiles have existed for nearly a century, their production and customisation has always remained industrial. This has meant that with the exception of the very well resourced, their end user, the designer, can only have indirect control over the sourcing and material properties of synthetic textiles.

Here we see the inherent strength of using bacterial cellulose as a lens though which we can see how these new materials and industries may look. While biomaterial companies, such as Spiber, Bolt and Modern Meadow have been successful in presenting biofabricated prototypes, these projects have required significant investment in infrastructure and expertise. When compared against other biofabricated materials, bacterial cellulose is easy to produce and can be grown at large scale with minimal infrastructure allowing for ample experimentation and prototyping at the “cottage industry” level. Having created a genetically modified K. rhaeticus that was capable of self-dyeing, we sought to explore its potential as a textile. We collaborated with the biodesigner Jen Keane to grow a genetically modified, self-dyeing, bacterial cellulose shoe upper. This work built upon Jen Keane's previous project, This is Grown, which is detailed in the Introduction.

This project, which came to be known as This is GMO was the result of many months of collaboration, in which we explored the path of combining Jen Keane's bacterial cellulose growing craft with the technicalities of growing genetically modified organisms in a laboratory context. To align with Jen Keane's previous success with large scale pellicles, we used a coconut water media supplemented with copper and L-tyrosine for growing K. rhaeticus ptyr1 in the shoe upper loom. Additionally, in order to scale up to such a large pellicle, and have it grow evenly, we grew a sequential series of K. rhaeticus ptyr1 pellicles, scaling from small volumes up to large volumes, until there was enough inoculum to inoculate the shoe upper loom. After 14 days, the K. rhaeticus ptyr1 pellicle was harvested, washed and placed into a development bath to undergo eumelanin development. The final melanated shoe upper pellicle was soaked in a 5% glycerol solution, which increased the pliability of the BC once dried. Finally, the pellicle was wrapped around a last and allowed to dry before being attached to a sole.

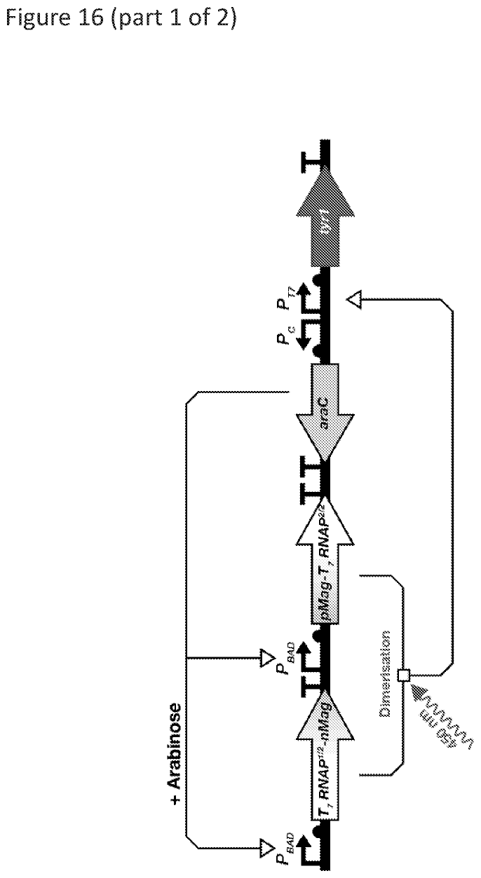

The experience of growing the melanated shoe upper presented some complications in pellicle growing and melanin development that were not met until growing at this scale. To prevent contamination the shoe loom was covered during growth, however this covering lead to the build-up of condensation that would drip down back onto the culture, causing the early pellicle to sink. This required a careful balancing of ventilation, protection from contamination and heat source placement to solve. Additionally, growing at this scale and for such an extended amount of time meant contamination was difficult to avoid. After 14 days of growth, colonies of mould could be observed growing on the pellicle and the growing process was stopped to prevent damage to the pellicle (FIG. 17).

After pellicle harvest, pellicles were normally placed into conical flask with a large excess of melanin development buffer and shaken at high speed. However, due to the size of this pellicle and its attachment to the loom it was not possible to move it to a larger container with an excess of development buffer. Instead, a higher concentration development buffer was made, with the PBS contents included at 10× the normal amount to provide additional buffering against the pellicle. When the pellicle was placed into the development bath the pH of the bath was checked over time. After the first 24 hours the development bath pH had dropped below 5 as a result of the acidifying action of K. rhaeticus. At this point the acidified development bath was drained and exchange with fresh buffer. We initially attempted a stationary melanin development (FIG. 17). After 24 hours however, the melanin production was patchy, and it was decided to place the pellicle into a incubator with shaking to increase the aeration. The final melanated shoe upper was the result of an additional 24 hours with this shaking (FIG. 17).

### Example 11—a Modular Optogenetic System for K. rhaeticus

The Opto-T7 system has the potential to place the expression of any gene or polynucleotide, that can be placed upstream of the T7 polymerase promoter, under the regulation of light. However, when all components are placed on one plasmid, it is difficult to successfully transform in to K. rhaeticus by electroporation due to the size of the plasmid. Additionally, attempts to alter the target protein expressed by the T7-opto system presents a difficult cloning challenge. For this reason, the inventors have made a more modular version of the T7-opto system, that separates the target gene onto a secondary plasmid, containing a spectinomycin resistance marker. The split T7-RNAP and araC regulatory genes are integrated into the K. rhaeticus chromosome by homologues recombination. By integrating the largest component of the optogenetic system—the split T7-RNAP—into the chromosome of K. rhaeticus, we aimed to increase the genetic stability of the system. Additionally, by placing the target gene onto a separate plasmid, we increase the ease of switching target genes.

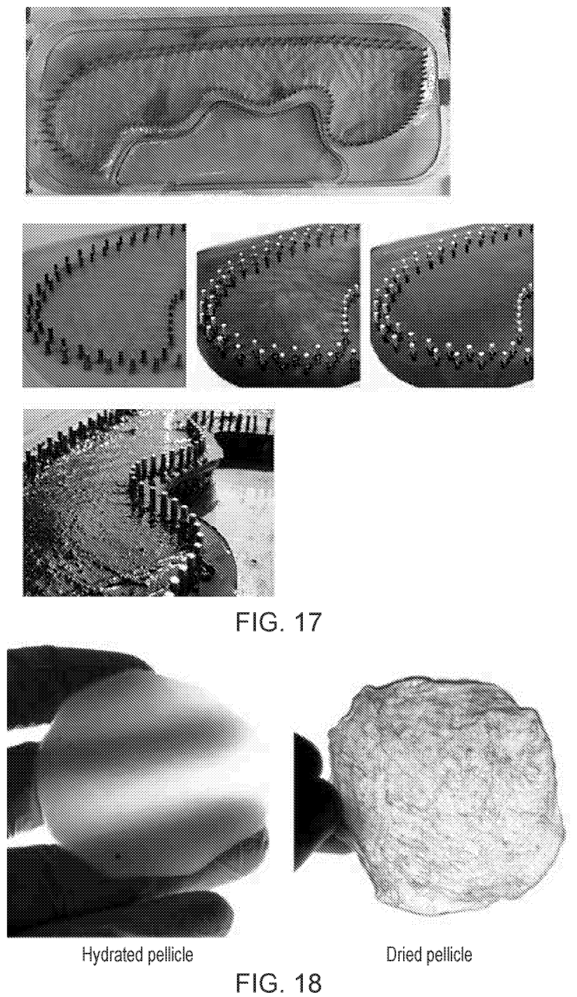

Five variations of the Opto-T7 RNAP system were integrated into the chromosome of K. rhaeticus, creating 5 separate strains of putative blue-light sensitive K. rhaeticus-Opto-T7RNAP*(563-F1), Opto-T7RNAP*(563-F2), Opto-T7RNAP*(69), Opto-T7RNAP*(563), Opto-T7RNAP(563-F1) (FIG. 19a). Nucleic acid sequences of the components of these systems is are set out in Table 1.

TABLE 1
Nucleotide sequences used in the modular optogenetic system

| | First nucleotide sequence | | | Second nucleotide sequence | | |
|---|---|---|---|---|---|---|
| | | | RNAP | | | RNAP |
| | RNAP | | first | | RNAP | second |
| | first | | half + | | second | half + |
| | half | nMag | nMag | pMag | half | pMag |
| opto- | SEQ ID | SEQ ID | SEQ ID | SEQ ID | SEQ ID | SEQ ID |
| T&RNAP*(563-F1) | NO: 3 | NO: 7 | NO: 9 | NO: 8 | NO: 4 | NO: 10 |
| opto- | SEQ ID | SEQ ID | SEQ ID | SEQ ID | SEQ ID | SEQ ID |
| T&RNAP*(563-F2) | NO: 3 | NO: 7 | NO: 9 | NO: 30 | NO: 4 | NO: 36 |
| opto- | SEQ ID | SEQ ID | SEQ ID | SEQ ID | SEQ ID | SEQ ID |
| T&RNAP*(69) | NO: 26 | NO: 7 | NO: 28 | NO: 44 | NO: 32 | NO: 38 |
| opto- | SEQ ID | SEQ ID | SEQ ID | SEQ ID | SEQ ID | SEQ ID |
| T&RNAP*(563) | NO: 3 | NO: 7 | NO: 9 | NO: 44 | NO: 4 | NO: 40 |
| opto- | SEQ ID | SEQ ID | SEQ ID | SEQ ID | SEQ ID | SEQ ID |
| T&RNAP(563-F1) | NO: 3 | NO: 7 | NO: 9 | NO: 8 | NO: 34 | NO: 42 |
| opto- | SEQ ID | SEQ ID | SEQ ID | SEQ ID | SEQ ID | SEQ ID |
| T&RNAP*(563-F1) | NO: 3 | NO: 7 | NO: 9 | NO: 8 | NO: 4 | NO: 10 |
| (OptoT7 RNAP) | | | | | | |

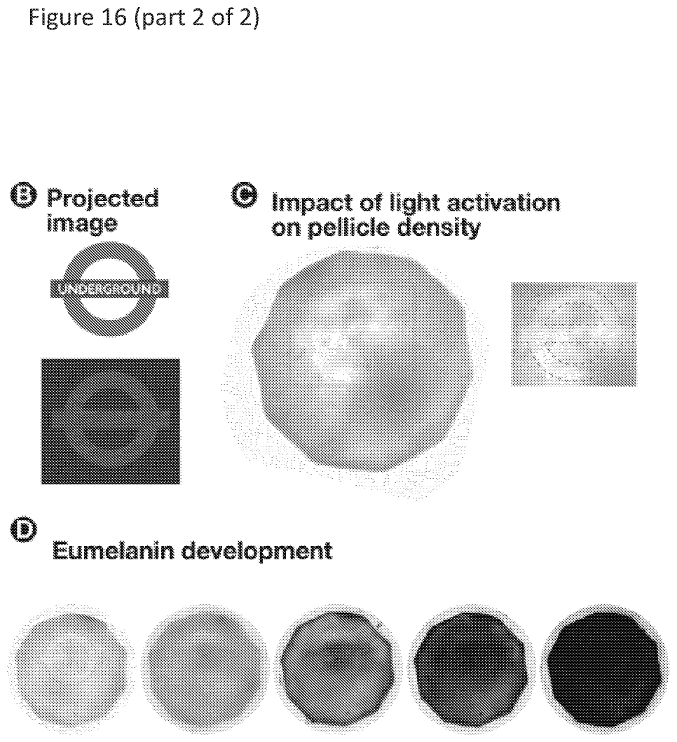

Two proteins, mCherry and Tyr1 were placed under a T7 promoter on a spectinomycin resistant pBBR1 plasmid from the KTK toolkit. This made two separate output plasmids, pT7_mCherry and pT7_tyr1. These were transformed into the Opto-T7-RNAP K. rhaeticus strains in order to test our ability to regulate gene expression with blue light (FIG. 18a).

The Opto-T7-RNAP K. rhaeticus strains carrying either the pT7_mCherry or pT7_tyr1 plasmids were pre-cultured in the dark in 96-well deep well plates with HS media, containing either 0, 1, 10 or 100 mg/ml of arabinose. After 18 hours, cells were split across two plates, diluted 1:2 Into fresh media with a matching arabinose concentration before placing one plate under blue light and the other in darkness (FIG. 19b). After 6 hours in the two lighting conditions at 30° C. and shaking, cells were assayed for either mCherry fluorescence or melanin production activity in a plate reader.

Opto-T7-RNAP K. rhaeticus integrated strains Opto-T7RNAP*(563-F1), Opto-T7RNAP*(563-F2), Opto-T7RNAP*(69), and Opto-T7RNAP*(563) achieved lower production of both mCherry and Tyr1 In light and dark growth conditions compared to the K. rhaeticus strain containing the original Opto-T7 polymerase plasmid (Plasmid_Opto-T7RNAP*(563-F2)). K. rhaeticus Opto-T7RNAP(563-F1), however, produced a much stronger absolute expression than the other integrated strains. K. rhaeticus Opto-T7RNAP(563-F1) was the only integrated strain that showed a response, similar in magnitude, to the Plasmid_Opto-T7RNAP*(563-F2)). K. rhaeticus Opto-T7RNAP(563-F1) increased mCherry production the most in response to blue light (7.4 fold (t0.5)) in HS-media containing 10 mg/mL arabinose. The best Tyr1 production increase in blue light (5.35 fold (±3.4)) came from 1 mg/mL arabinose.

### Example 12—Spatial Pigmentation of Pellicles Using a Modular Optogenetic System for K. rhaeticus

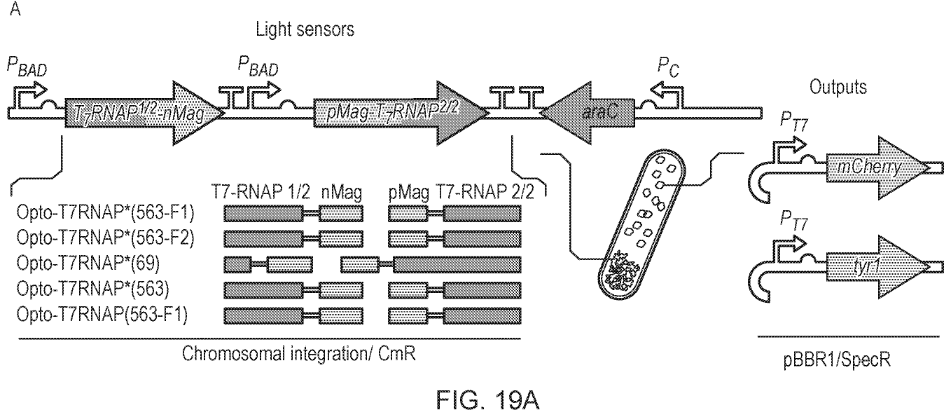

We then assessed patterned melanin production in a pellicle produced by the K. rhaeticus Opto-T7RNAP(563-F1) strain containing pT7_Tyr1 plasmid. A custom rig was built to hold a commercial LED projector (ViewSonic M1) that could project an image or movie over a polypropylene pellicle culture chamber (FIG. 20a). This custom rig allowed the inventors to expose pellicles with a size of up to 500×300 mm to light. The rig was draped with black-out fabric to remove outside light and a small heater fitted inside to maintain temperatures at about 30° C.

A timelapse movie was designed to test how long a given pellicle would need to be exposed to light before an identifiable change in pigmentation could be observed. The total timelapse length was 80 hours (FIG. 20b).

To grow the pellicle, the Opto-T7RNAP(563-F1) strain with the pT7_Tyr1 plasmid was inoculated into 1 L of coconut water media containing 1% apple cider vinegar, 10 mg/ml of arabinose, 1% ethanol, 0.5 g/L tyrosine and 20 μM CuSO₄; and was incubated in dark, stationary conditions at 30° C. inside the optogentic rig. After 8 days in these conditions, once pellicle growth covered the media surface, the projector was turned on and the pellicle was exposed for 80 hours. After this exposure, the pellicle was placed gently into 2 L a development solution containing 10×PBS with 1 g/L tyrosine and 20 μM CuSO₄, and left at 30° C. to develop slowly over 2 days. The present experiment was conducted using a larger pellicle (150 mm×300 mm) than the pellicle used in Examples 6-9, which had a size below 150×150 mm. The inventors found that larger pellicles contain higher amounts of acetic and gluconic acid than smaller pellicles, and therefore required increased buffering capacity. Accordingly, 10×PBS was used in this experiment to improve the buffering of development solution.

The final pellicle shows some identifiable pigment patterns, such as the two roundel images, on the left side of the pellicle (FIG. 20c). A densitometry scan of the pellicle revealed at least 6 blocks had been successfully printed along the top row, suggesting a minimal exposure time of ˜32 hours.

### Example 13—Comparative Analysis of Development Buffer Conditions for Melanin Production

Tyrosinase catalysed melanin production is known to be sensitive to environmental conditions, such as temperature, pH and substrate concentration. Understanding how these conditions effect the rate and accumulation of melanin produced is essential to optimising the production of melanated bacterial cellulose from K. rhaeticus. To investigate these conditions, cultured K. rhaeticus tyr1 cells were subjected to a variety of development buffer conditions and the melanin production of these cells measured by optical density in a microtiter plate setup.

As set out in Effect of temperature on melanin production, the rate of melanin accumulation was maximised when K. rhaeticus tyr1 cells were placed in development buffer at 45° C. Accordingly, to improve experimental efficiency, the following melanin production assays were conducted at 45° C.

### Methods—Cell Preparation

Komagataeibacter rhaeticus bacteria were grown in Hestrin-Schramm (HS) media (Hestrin and Schramm, 1954, Biochem. J., 58(2), pp. 345-352. doi: 10.1042/bj0580345.). HS media content: yeast extract 0.5% (w/v), peptone 0.5% (w/v), glucose 2% (w/v), Na₂HPO₄ 0.27% (w/v), citric acid 0.15% (w/v), adjusted to pH 5.8-6. To prepare K. rhaeticus cells for the melanin production assays, HS media is inoculated with K. rhaeticus and grown in shaking conditions at 30° C. with 2% cellulase to prevent pellicle formation. Once the culture is turbid, it is used to inoculate 100 mL of HS media with 2% cellulase in a flask at a ratio of 1:100. This culture is grown at 30° C. with shaking until OD₆₀₀ reaches 0.8-2. The culture is then centrifuged, and the resulting pellet washed twice, to remove spent media, and adjusted to an OD₆₀₀ of ˜1.

### The Effect of DH on Melanin Production

An acetate-borate-phosphate buffer (0.04 M boric acid, 0.04M phosphoric acid, 0.04 acetic acid) containing 10 μM CuSO₄ and 0.5 g/L L-Tyrosine was prepared. This buffer was then placed into separate tubes and NaOH and HCl were added to each tube to produce melanin development solutions at pH values from 3-11. K. rhaeticus tyr1 cells at an OD₆₀₀ of 1 were mixed at 1:5 ratio with these buffers in a 384 well microtiter plate. This plate was shaken with shaking at 45° C. and the optical density of the solution was measured at 405 nm to monitor the production of melanin.

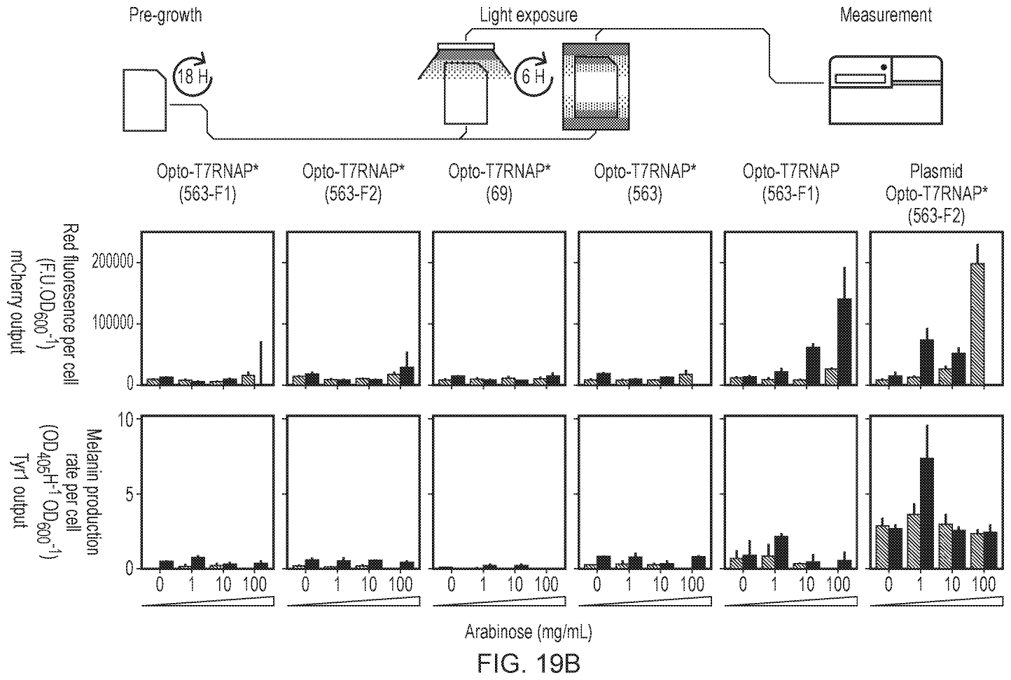

Melanin production was catalysed most quickly in alkaline conditions—i.e., a pH between 7 and 14—and total melanin yield was maximised between pH 7 and pH 8 (FIG. 21).

### The Effect of PBS Buffer Concentration on Melanin Production

A 20× stock of phosphate-buffered saline was produced containing 2.74 M NaCl, 45 mM KCL, 200 mM Na₂HPO₄, 36 mM KH₂PO₄, 10 μM CuSO₄ and 0.5 g/L L-Tyrosine. This 20×PBS was then diluted with ddH₂O containing 10 μM CuSO₄ and 0.5 g/L L-tyrosine to produce melanin development solutions containing a range of PBS concentrations: 20×, 10×, 9×, 8×, 7×, 6×, 5×, 4×, 3×, 2×, 1×, and 0×. K. rhaeticus tyr1 cells at an OD₆₀₀ of 1 were mixed at 1:5 ratio with these buffers in a 384 well microtiter plate. This plate was shaken with shaking at 45° C. and the optical density at 405 nm was measured to monitor the production of melanin.

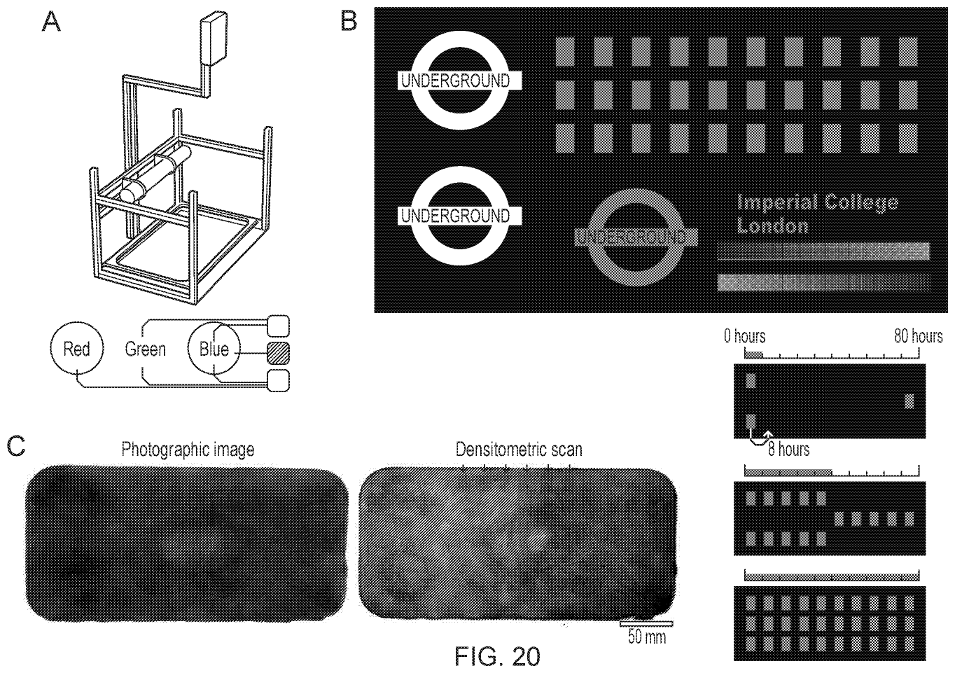

K. rhaeticus required buffered conditions to produce melanin (FIG. 22). Melanin production was maximised at a 1× concentration of PBS and was possible in higher concentrations of PBS.

### The Effect of Different Metal Ions on Melanin Production

Metal salts (copper sulphate, nickel chloride, cobalt chloride, zinc chloride) at a concentration of 10 μM were added to a 10×PBS solution at pH 7.4 containing 0.5 g/L L-tyrosine. K. rhaeticus tyr1 cells at an OD₆₀₀ of 1 were mixed at a 1:5 ratio with these buffers in a 384 well microtiter plate. This plate was shaken with shaking at 45° C. and the optical density at 405 nm was measured to monitor the production of melanin.

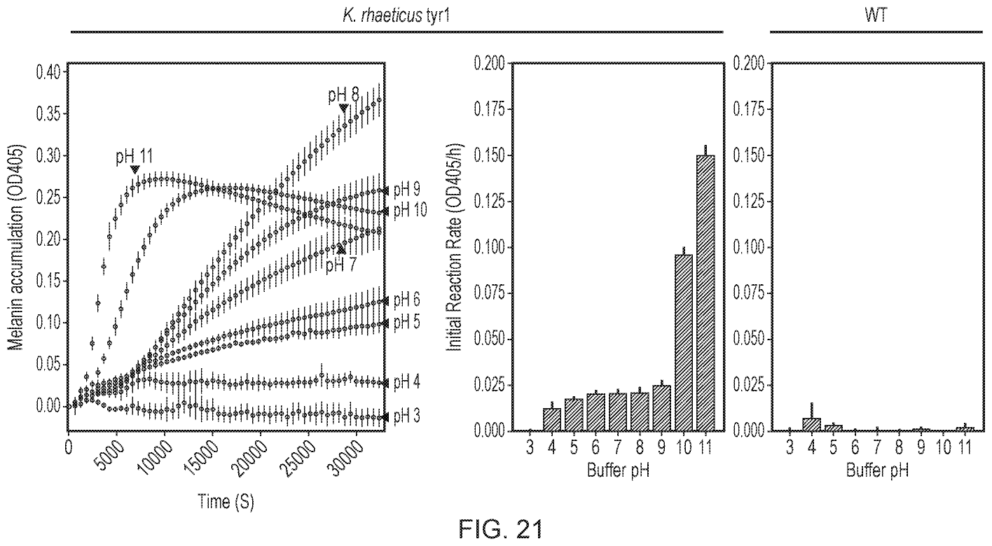

For melanin production to occur, L-tyrosine must be present in the development buffer. It was not essential for copper to be present in the development buffer, however the production rate was improved in the presence of 10 μM CuSO₄. Melanin production rate in development solutions containing either Zinc, Nickel, or Cobalt Ions was equivalent to the production rate in development buffer containing only L-tyrosine. See FIG. 23.

### The Effect of Conner (II) (Cu²⁺) Concentration on Melanin Production

10×PBS solutions at pH 7.4 with 0.5 g/L L-tyrosine were prepared containing a range of CuSO₄ concentrations from 0 to 1280 μM. K. rhaeticus tyr1 cells at an OD₆₀₀ of 1 were mixed at 1:5 ratio with these buffers in a 384 well microtiter plate. This plate was shaken with shaking at 45° C. and the optical density at 405 nm was measured to monitor the production of melanin.

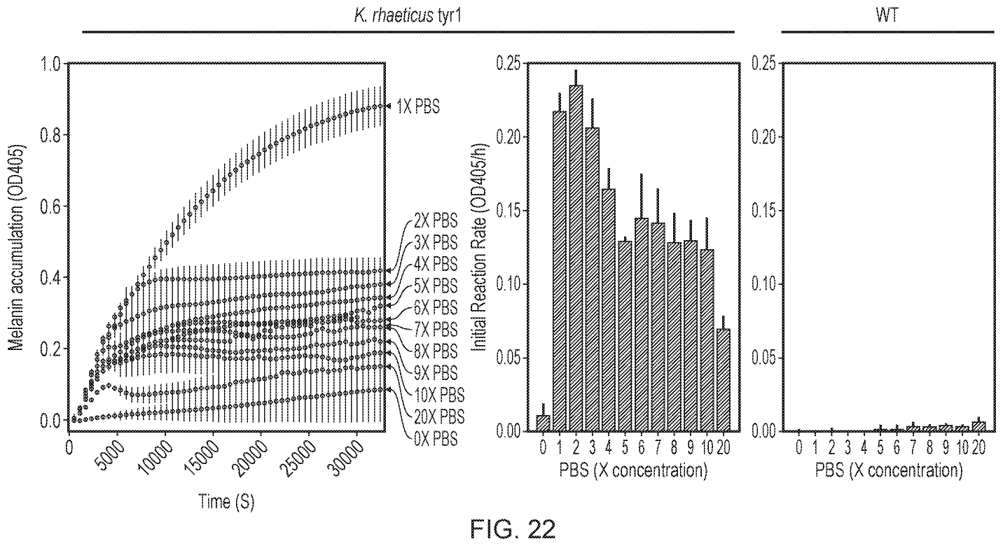

Melanin production rate was highest when 20 μM of CuSO₄ was present in the development buffer. The data also showed that a broad range of CuSO₄ concentrations could be used to catalyse melanin synthesis. See FIG. 24.

### Effect of Tyrosine Concentration on Melanin Production

10×PBS solutions at pH 7.4 with 10 μM CuSO₄ were prepared containing a range of L-Tyrosine concentrations from 0 to 1 g/L-concentrations above 1 g/L produced solutions too cloudy to accurately measure melanin production. K. rhaeticus tyr1 cells at an OD₆₀₀ of 1 were mixed at 1:5 ratio with these buffers in a 384 well microtiter plate. This plate was shaken with shaking at 45° C. and the optical density at 405 nm was measured to monitor the production of melanin.

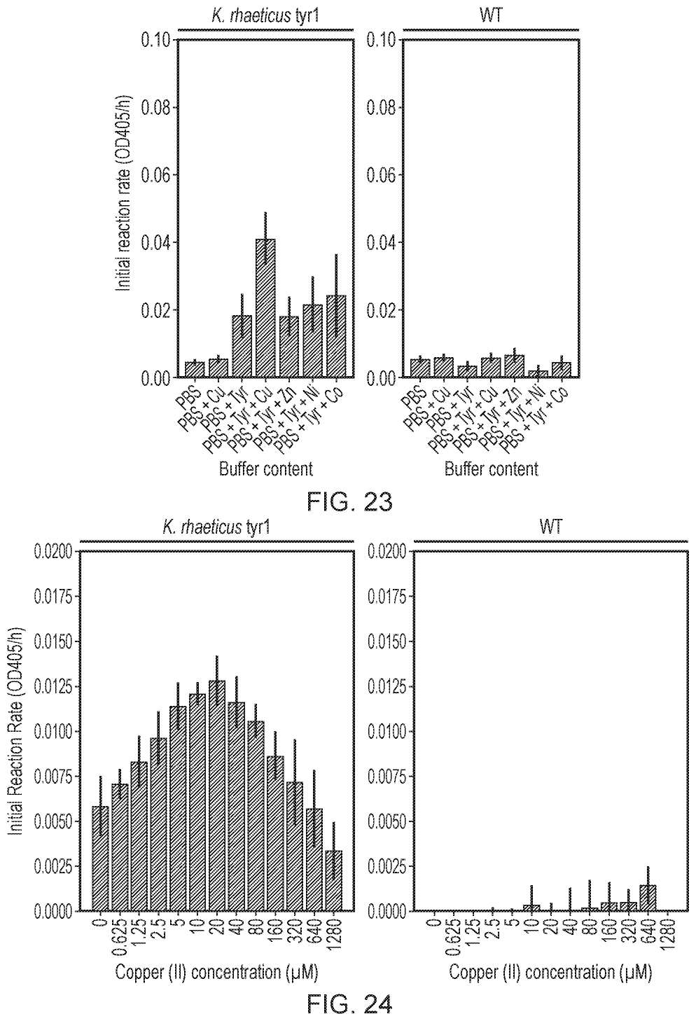

Melanin production rate increased as tyrosine concentrations were increased; and was optimised when L-tyrosine concentrations in the development buffer were at or above 0.5 g/L. See FIG. 25.

### Effect of Temperature on Melanin Production

A 10×PBS solution at pH 7.4 with 10 μM CuSO₄ and 0.5 g/L L-Tyrosine was prepared. K. rhaeticus tyr1 cells at an OD₆₀₀ of 1 were mixed at 1:5 ratio with these buffers in a 96 well clear PCR plate. The plate was placed in a 96 well thermocycler with variable heat setting from 25-50° C. Every 20 mins, samples were taken up until 140 mins had passed. The optical density of each well at 405 nm was measured.

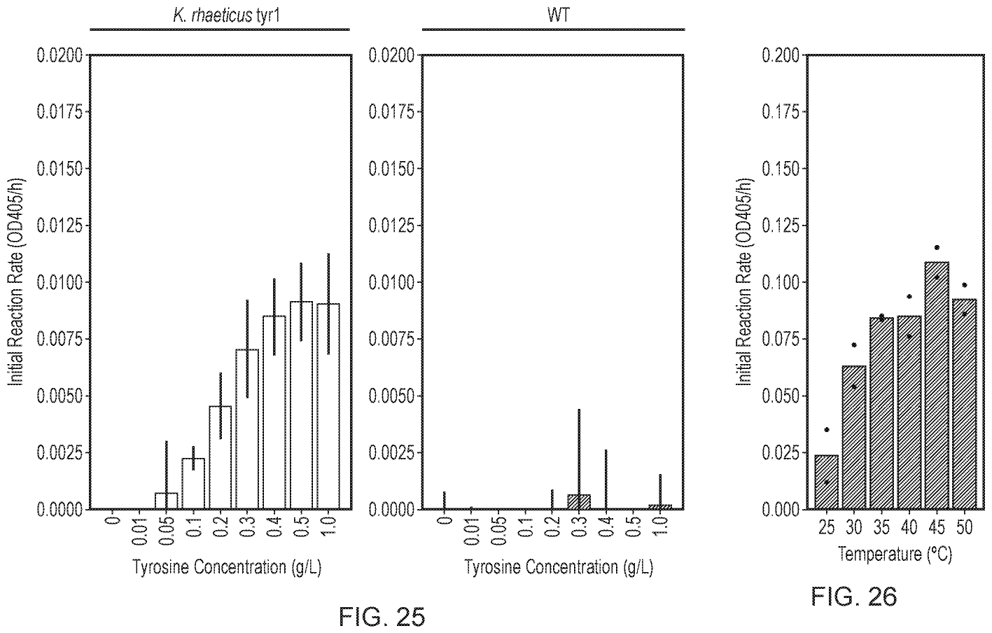

Melanin production rate increased as the temperature at which the reaction was conducted increased from 25° C.; and was optimised at 45° C. See FIG. 26.

The foregoing embodiments, Instances, and examples are applicable to any of the aspects of the present disclosure and should be construed as such.

While the present disclosure has been described in terms of various aspects, embodiments, and examples, it is understood that variations, improvements, and equivalents will occur to the person skilled in the art. Such variations, improvements, and equivalents are contemplated by the present disclosure and fall within the scope of the matter disclosed and claimed herein.

1. A method for producing melanated bacterial cellulose, wherein the method comprises exposing a cellulose pellicle that comprises tyrosinase to a development solution, wherein the development solution:
is at a pH of between 6 and 8.5;
comprises L-tyrosine and/or L-cysteine and/or L-cystine;
and optionally comprises metal ions with an oxidation state of 2+;
optionally wherein the cellulose pellicle was produced by bacterial cells that express tyrosinase.

2. A method for producing melanated bacterial cellulose, wherein the method comprises:
a) culturing a cellulose producing bacteria under conditions so as to allow a pellicle to form
wherein the bacteria express tyrosinase; and
b) exposing the pellicle formed in a) to a development solution;
wherein the development solution:
is at a pH of between 6 and 8.5;
comprises L-tyrosine and/or L-cysteine and/or L-cystine;
and optionally
comprises metal ions with an oxidation state of 2+;

3. The method according to claim 2 wherein the conditions that allow a pellicle to form comprise culturing the bacteria:
a) at a pH of:
between 3-7, optionally a pH of between 3.25 and 6.75, 3.5 and 6.5, 3.5 and 6.25, 3.75 and 6, 4 and 5.75, 4.25 and 5.5, 4.5 and 5.25; pH 5.8; and/or
at least 3 but less than or equal to pH 7, for example at least 3, 3.25, 3.5, 3.75, 4, 4.25, 4.5, 4.75, 5, 5.25, 5.5, 5.75, 5.8, 6, 6.25, 6.5, 6.75, but less than or equal to pH 7;
and/or
b) in culture media that is:
i) HS media; or
ii) Coconut water media

4. The method according to any of claim 2 or 3 wherein (b) is performed after the pellicle formed in (a) Is harvested.

5. The method according to any of the preceding claims wherein the bacterial cells:
a) are capable of producing bacterial cellulose;
b) express all of bcsA, bcsD, bscC and bscD;
c) belong to a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes;
d) are selected from the group comprising or consisting of: Komagaeibacter rhaeticus; Komagaeibacter xylinus, Komagaeibacter hansenii, Komagaeibacter medellinensis, Komagaelbacter europaeus, Komagaeibacter maltaceti, Komagaelbacter pomaceti, Komagaeibacter oboediens, or Komagaeibacter saccharivoans;
e) are selected from the group comprising or consisting of:
i) a strain of Komagaelbacter rhaeticus selected from the group comprising or consisting of: Komagaeibacter rhaeticus iGEM. Komagaeibacter rhaeticus AF; Komagaeibacter rhaeticus LMG22126; or
ii) Gluconacetobacter xylinus CGMCC 2995;
and/or
f) are Komagaeibacter rhaeticus IGEM cells.

6. The method according to any of the preceding claims wherein the metal ions with an oxidation state of 2+ are selected from:
a) Cu²⁺, Zn²⁺, Be²⁺, Mg²⁺, Ca²⁺, Cr²⁺, Mn²⁺, Co²⁺ or Ni²⁺;
b) Cu²⁺, Zn²⁺; or
c) Cu²⁺.

7. The method according to any of the preceding claims wherein the development solution comprises:
a) a water-soluble copper (II) salt, optionally comprises CuSO₄ or CuCl₂, optionally comprises:
i) at least 2 μM CuSO₄, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuSO₄; and/or
between 2 μM CuSO₄ and 20 μM CuSO₄; and/or
less than 20 μM, optionally less than 17.5 μM, 15 μM, 12.5 μM, 10 μM, 7.5 μM, 5 μM, 4 μM, 3 μM, or less than 2 μM; and/or
between 20 μM and 160 μM CuSO₄; and/or
less than 160 μM, optionally less than 140 μM, 120 μM, 100 μM, 80 μM, 60 μM, 40 μM, or less than 20 μM CuSO₄;
and/or
ii) at least 2 μM CuCl₂, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuCl₂; and/or
between 2 μM CuCl₂ and 20 μM CuCl₂; and/or
less than 20 μM, optionally less than 17.5 μM, 15 μM, 12.5 μM, 10 μM, 7.5 μM, 5 μM, 4 μM, 3 μM, or less than 2 μM; and/or
between 20 μM and 160 μM CuCl₂; and/or
less than 160 μM, optionally less than 140 μM, 120 μM, 100 μM, 80 μM, 60 μM, 40 μM, or less than 20 μM CuCl₂;
and/or
b) at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L tyrosine; and/or
less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine;
and/or
c) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
between 10 g/L and 300 g/L cysteine; and/or
less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
and/or
d) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L L-cystine; and/or
less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine.
optionally wherein the development solution is at a pH of:
at least 6, optionally at least 6.25, 6.5, 6.75, 7, 7.25, 7.4, 7.5, 7.75, 8, 8.25 or at least 8.5; and/or
between 6 and 8.5, optionally between 6.25 and 8.25, 6.5 and 8, 6.25 and 7.75, 6.5 and 7.5, 6.75 and 7.25; or
7.4.

8. The method according to any of the preceding claims wherein the development solution comprises:
a) PBS at pH 7.4;
b) 10 μM CuSO₄ or 20 μM CuSO₄; and
c) 0.5 g/L L-tyrosine or 1 g/L L-tyrosine, and/or 1 g/L L-cysteine and/or 0.4 g/L L-cystine.

9. The method according to any of the preceding claims wherein the method comprises a further step of:
(c) sterilising the pellicle following incubation in the development solution, optionally wherein the sterilisation is selected from the group comprising or consisting of:
i) autoclaving;
ii) heating; and/or
iii) desiccation, optionally with 70% ethanol.

10. The method according to any of the preceding claims wherein the tyrosinase:
a) Is a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences; and/or
b) is operably linked to Anderson promoter J23104 [SEQ ID NO: 16] and RBS B0034 [SEQ ID NO: 17]; optionally is operably linked to Anderson promoter J23104 and RBS B0034 that comprises or consists of a sequence with at least 80%, 82%, 84%, 86%, 88%, 90%, 92%, 94%, 96%, 98%, 99% or 100% sequence identity to any of the above sequences.

11. The method according to any of the preceding claims wherein the cells are cultured in a culture medium that:
i) is Hestrin and Schramm (HS) medium;
ii) is supplemented with glucose, optionally at 2% (w/v); and/or
iii) is buffered to a pH of 5.8.

12. A nucleic acid comprising a regulatory sequence and a sequence that encodes a tyrosinase enzyme wherein the regulatory sequence comprises Anderson promoter J23104 and RBS B0034, optionally wherein:
a) the sequence that encodes a tyrosinase enzyme encodes a bacterial tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences; and/or
b) the nucleic acid is a circular nucleic acid, optionally is a circular nucleic acid selected from the group consisting of a plasmid, a bacterial artificial chromosome, a phagemid, a cosmid, a yeast artificial chromosome, a human artificial chromosome, a viral vector, optionally
wherein the circular nucleic acid, optionally a plasmid, further comprises an origin of replication; and/or
c) wherein any one or more of the nucleic acids further comprises a selectable marker; and/or
d) the nucleic acid is integrated into the genome of a cell, optionally a bacterial cell.

13. A cell comprising the nucleic acid according to claim 12 wherein the cell is:
i) a bacterial cell that is capable of producing bacterial cellulose;
ii) a bacterial cell that expresses all of bcsA, bcsD, bscC and bscD;
iii) a bacterial cell of a genus selected from the group comprising or consisting of:
Komagataeibacter, Escherichia, Gluconacetobacter, Acetobacter, Sarcina, Agrobacterium, Azotobacter, Rhizobium, Pseudomonas, Salmonella and Alcaligenes;
iv) a bacterial cell selected from the group comprising or consisting of: Komagaelbacter rhaeticus; Komagaeibacter xylinus, Komagaelbacter hansenii, Komagaelbacter medellinensis, Komagaelbacter europaeus, Komagaelbacter maltaceti, Komagaelbacter pomaceti, Komagaelbacter oboediens, or Komagaelbacter saccharivoans;
v) a bacterial cell selected from the group comprising or consisting of:
a) a strain of Komagaeibacter rhaeticus selected from the group comprising or consisting of:
Komagaelbacter rhaeticus IGEM, Komagaeibacter rhaeticus AF1;
Komagaelbacter rhaeticus LMG22126; or
b) Gluconacetobacter xylinus CGMCC 2995; or
vi) a bacterial cell that is a Komagaeibacter rhaeticus iGEM cell.

14. An optogenetic expression system for use in bacteria of the genus Komagataeibacter, comprising:
(a) A first nucleic acid comprising a first nucleotide sequence that encodes a first polypeptide, wherein the first polypeptide comprises:
i) a first domain that comprises a first portion of a heterologous split-polymerase or a split-transcription factor; and
ii) a second domain that comprises a first light-inducible dimerization domain;
(b) A second nucleic acid comprising a second nucleotide sequence that encodes a second polypeptide, wherein the second polypeptide comprises:
i) A first domain that comprises a second portion of a heterologous split-polymerase or a split-transcription factor; and
ii) A second domain that comprises a second light-inducible dimerization domain;
and
(c) A third nucleic acid comprising a third nucleic acid sequence that encodes a target protein or RNA to be expressed operably linked to a target promoter;
and wherein the first light-inducible dimerization domain and the second light-inducible dimerization domain are capable of dimerising with one another upon exposure to light of a dimerization wavelength to form a functional heterologous polymerase or a functional transcription factor capable of transcribing or initiating transcription from the target promoter,
and wherein the target promoter is recognised by the functional heterologous polymerase or functional transcription factor so as to drive transcription of the third nucleic acid sequence that encodes a target protein or RNA.

15. The optogenetic expression system according to claim 14 wherein:
a) the target promoter is a heterologous promoter;
b) the first nucleic acid, the second nucleic acid and the third nucleic acid are:
i) all part of the same nucleic acid molecule; or
ii) are different nucleic acid molecules;
or
c) the first nucleic acid and the second nucleic acid are part of the same nucleic acid molecule, and the third nucleic acid is part of a different nucleic acid molecule;
and/or
d) i) the heterologous split-polymerase is a split-T7 polymerase or the functional heterologous polymerase is a T7 polymerase; or
ii) the split-transcription factor is a split-LuxR, optionally wherein the target promoter comprises a LuxR binding site;
and/or
e) the first nucleic acid comprises a first promoter operably linked so as to drive expression of the first polypeptide; and/or
f) the second nucleic acid comprises a second promoter operably linked so as to drive expression of the second polypeptide.

16. The optogenetic expression system according to any of claim 14 or 15, wherein:
the first portion of the heterologous split-polymerase comprises or consists of a sequence of at least 70%, at least 75%, at least 80%, at least 85%, at least 90%, at least 95%, at least 96%, at least 97%, at least 98%, or at least 99% identical to SEQ ID NO: 1 and/or SEQ ID NO: 27, or 100% Identical to SEQ ID NO: 1 and/or SEQ ID NO: 27; and/or
the second portion of the heterologous split-polymerase comprises or consists of a sequence of at least 70%, at least 75%, at least 80%, at least 85%, at least 90%, at least 95%, at least 96%, at least 97%, at least 98%, or at least 99% identical to SEQ ID NO: 2, SEQ ID NO: 33, and/or SEQ ID NO: 35, or 100% identical to SEQ ID NO: 2, SEQ ID NO: 33, and/or SEQ ID NO: 35; and/or
the first portion of the heterologous split-polymerase is encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 3 and/or SEQ ID NO: 26, or 100% identical to SEQ ID NO: 3 and/or SEQ ID NO: 26; and/or
the second portion of the heterologous split-polymerase is encoded by a DNA sequence that comprises or consists of a sequence of at least 70%, at least 75%, at least 80%, at least 85%, at least 90%, at least 95%, at least 96%, at least 97%, at least 98%, or at least 99% identical to SEQ ID NO: 4, SEQ ID NO: 32, and/or SEQ ID NO: 34, or 100% identical to SEQ ID NO: 4, SEQ ID NO: 32, and/or SEQ ID NO: 34.

17. The optogenetic expression system according to any of claims 14-16 wherein:
the first light-inducible dimerization domain is a LOV dimerization domain and the second light-inducible dimerisation domain is a LOV dimerisation domain;
the first light-inducible dimerization domain is an nMag dimerization domain and the second light-inducible dimerisation domain is a pMag dimerisation domain;
the first light-inducible dimerization domain is a pMag dimerization domain and the second light-inducible dimerisation domain is an nMag dimerisation domain;
the first light-inducible dimerization domain is a VVD dimerization domain and the second light-Inducible dimerization domain is a VVD dimerization domain;
the first light-inducible dimerization domain is a LOVtrap dimerization domain and the second light-inducible dimerisation domain is an LOVtrap dimerisation domain;
the first light-inducible dimerization domain is a VfAU1-LOV dimerization domain and the second light-inducible dimerisation domain is a VfAU1-LOV dimerisation domain;
the first light-inducible dimerization domain is a NgPA1-LOV dimerization domain and the second light-inducible dimerisation domain is a NgPA1-LOV dimerisation domain;
the first light-inducible dimerization domain is a OdPA1-LOV dimerization domain and the second light-inducible dimerisation domain is a OdPA1-LOV dimerisation domain;
the first light-inducible dimerization domain is a AsLOV2 dimerization domain and the second light-inducible dimerisation domain is an PDZ dimerisation domain;
the first light-inducible dimerization domain is a PDZ dimerization domain and the second light-inducible dimerisation domain is a AsLOV2 dimerisation domain;
the first light-inducible dimerization domain is a AtCry2 dimerization domain and the second light-inducible dimerisation domain is a AtCry2 dimerisation domain;
the first light-inducible dimerization domain is a PhyB dimerization domain and the second light-inducible dimerisation domain is a PIF dimerisation domain;
the first light-inducible dimerization domain is a PIF dimerization domain and the second light-inducible dimerisation domain is a PhyB dimerisation domain;
the first light-inducible dimerization domain is a Cph1 dimerization domain and the second light-inducible dimerisation domain is a Cph1 dimerisation domain; or
the first light-inducible dimerization domain is a CBD dimerization domain and the second light-inducible dimerisation domain is a CBD dimerisation domain;
optionally wherein:
the nMag dimerization domain comprises or consists of a sequence that is at least 70%, at least 75%, at least 80%, at least 85%, at least 90%, at least 95%, at least 96%, at least 97%, at least 98%, or at least 99% identical to SEQ ID NO: 5 or 100% identical to SEQ ID NO: 5; and/or
the pMag dimerization domain comprises or consists of a sequence that is at least 70%, at least 75%, at least 80%, at least 85%, at least 90%, at least 95%, at least 96%, at least 97%, at least 98%, or at least 99% identical to SEQ ID NO: 6, SEQ ID NO: 31, and/or SEQ ID NO: 45, or 100% identical to SEQ ID NO: 6, SEQ ID NO: 31, and/or SEQ ID NO: 45
and/or
the nMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% identical to SEQ ID NO: 7 or 100% identical to SEQ ID NO: 7 and/or
the pMag dimerization domain is encoded by a DNA sequence that comprises or consists of a sequence that is at least 70%, at least 75%, at least 80%, at least 85%, at least 90%, at least 95%, at least 96%, at least 97%, at least 98%, or at least 99% identical to SEQ ID NO: 8, SEQ ID NO: 30, and/or SEQ ID NO: 44, or 100% identical to SEQ ID NO: 8, SEQ ID NO: 30, and/or SEQ ID NO: 44;
and/or
the first nucleic acid sequence comprises or consists of a nucleotide sequence having at least 70%, at least 75%, at least 80%, at least 85%, at least 90%, at least 95%, at least 96%, at least 97%, at least 98%, or at least 99% identical to any of SEQ ID NO: 9, 10, 11 12, 28, 36, 38, 40, or 42.

18. The optogenetic expression system according to any of claims 14-17 wherein the first dimerization domain and the second dimerization domain are substantially incapable of dimerization in the absence of light of the dimerization wavelength, optionally wherein the dimerization wavelength is about 400 nm to 500 nm;
optionally a wavelength of between 400 nm and 500 nm, optionally 450 nm.

19. The optogenetic expression system according to any of claims 14-18 wherein:
a) the third nucleotide sequence is capable of being transcribed into mRNA, optionally wherein the mRNA is capable of being translated into a polypeptide;
b) the third nucleotide sequence encodes a polypeptide; and/or
c) the third nucleotide sequence encodes a polypeptide that:
i) is involved in the biosynthesis of a pigment visible to the naked eye or in the biosynthesis of a molecule that emits light;
ii) encodes a protein that emits light or is a pigment.

20. The optogenetic expression system according to claim 19 wherein the polypeptide that is involved in the biosynthesis of a pigment visible to the naked eye is an enzyme necessary for the formation of melanin,
optionally wherein expression of the polypeptide that is involved in the biosynthesis of a pigment visible to the naked eye results in the formation of the pigment,
optionally wherein the melanin is selected from the group comprising eumelanin, pheomelanin, neuromelanin and allomelanin, optionally wherein the polypeptide is a bacterial tyrosinase,
optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences; and/or
wherein the third nucleotide sequence comprises the coding sequence for Tyr1, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.

21. The optogenetic expression system according to any of claims 14-20 wherein the protein that emits light emits light emits light of a second wavelength when exposed to light of a first wavelength, optionally wherein the protein that emits light is a fluorescent protein, optionally selected from the group comprising or consisting of:
mCherry, GFP, mScarlet, mRFP, cjBlue, gfasPurple, eforRed, spisPink

22. The optogenetic expression system according to any of claims 14-21 wherein the first promoter and/or the second promoter are inducible promoters or are constitutive promoters.

23. The optogenetic expression system according to any of claims 14-22:
a) further comprising a fourth nucleic acid sequence that encodes a heterologous protein required for inducible expression from the first and/or second promoter, optionally wherein the heterologous protein is a transcriptional regulator; and/or
b) wherein the first and/or second promoter is selected from the group comprising or consisting of:
P_{BAD} [SEQ ID NO: 18];
pLux [SEQ ID NO: 19];
pTet [SEQ ID NO: 20]; or
pLac [SEQ ID NO: 21];
optionally wherein the promoter comprises or consists of a sequence with at least 80%, 82%, 84%, 86%, 88%, 90%, 92%, 94%, 96%, 98%, 99% or 100% sequence identity to any of the above sequences; and/or
c) wherein the first and second promoter are both inducible promoters, optionally selected from the following inducible systems:
the pBAD promoter [SEQ ID NO: 18] induced by arabinose in the presence of the transcriptional regulator araC [SEQ ID NO: 22];
the pLux promoter [SEQ ID NO: 23] induced by Acyl Homoserine Lactone (AHL) in the presence of the transcriptional regulator LuxR [SEQ ID NO: 23];
the pTet promoter [SEQ ID NO: 20] induced by Anhydrotetracycline (ATc) in the presence of the transcriptional regulator TetR [SEQ ID NO: 24]; or
the pLac promoter [SEQ ID NO: 21] induced by IPTG in the presence of the transcriptional regulator LacI [SEQ ID NO: 25];
optionally wherein:
the first promoter is pBAD and the second promoter is pBAD;
the first promoter is pLUX and the second promoter is pLUX;
the first promoter is pTet and the second promoter is pTet;
the first promoter is pLac and the second promoter is pLac;
optionally wherein the promoter comprises or consists of a sequence with at least 80%, 82%, 84%, 86%, 88%, 90%, 92%, 94%, 96%, 98%, 99% or 100% sequence identity to [SEQ ID NO: 18], [SEQ ID NO: 19], [SEQ ID NO: 20], [SEQ ID NO: 21], and
optionally wherein the transcriptional regulator comprises or consists of a sequence with at least 80%, 82%, 84%, 86%, 88%, 90%, 92%, 94%, 96%, 98%, 99% or 100% sequence identity to [SEQ ID NO: 22], [SEQ ID NO: 23], [SEQ ID NO: 24], [SEQ ID NO: 25]; and/or
d) wherein the first and second promoters are both inducible promoters that are induced by the same inducer.
optionally wherein the first and second promoters are both induced by arabinose; and/or
e) wherein the first and second promoters comprise the P_{BAD} promoter sequence, optionally comprise or consist of [SEQ ID NO: 18] or a sequence that has at least 80%, 82%, 84%, 86%, 88%, 90%, 92%, 94%, 96%, 98%, 99% or 100% sequence identity to [SEQ ID NO: 18].

24. The optogenetic expression system according to claims 14-23 wherein:
a) the fourth nucleic acid sequence encodes a transcriptional regulator selected from the group comprising or consisting of:
araC [SEQ ID NO: 22] or a sequence with at least 80%, 82%, 84%, 86%, 88%, 90%, 92%, 94%, 96%, 98%, 99% or 100% sequence identity to [SEQ ID NO: 22];
LuxR [SEQ ID NO: 23] or a sequence with at least 80%, 82%, 84%, 86%, 88%, 90%, 92%, 94%, 96%, 98%, 99% or 100% sequence identity to [SEQ ID NO: 23];
TetR [SEQ ID NO: 24] or a sequence with at least 80%, 82%, 84%, 86%, 88%, 90%, 92%, 94%, 96%, 98%, 99% or 100% sequence identity to [SEQ ID NO: 24];
LacI [SEQ ID NO: 25] or a sequence with at least 80%, 82%, 84%, 86%, 88%, 90%, 92%, 94%, 96%, 98%, 99% or 100% sequence identity to [SEQ ID NO: 25];
b) the fourth nucleic acid sequence encodes a heterologous protein required for inducible expression from the first and/or second promoter, optionally wherein the heterologous protein is a transcriptional regulator, optionally
wherein the transcriptional regulator is araC; and/or
c) any of the first nucleotide sequence, the second nucleotide sequence, the third nucleotide sequence, and/or the fourth nucleotide sequence are operably linked to an enhancer sequence, a terminator sequence, a repressor sequence, an operator sequence and/or a sigma factor binding site.

25. The optogenetic expression system according to any of claims 14-24 wherein:
a) the first nucleic acid, the second nucleic acid, the third nucleic acid and optional fourth nucleic acid are:
i) all part of the same nucleic acid molecule; or
ii) are different nucleic acid molecules;
or
b) the first nucleic acid, second nucleic acid, and optional fourth nucleic acid are part of the same nucleic acid molecule, and the third nucleic acid is part of a different nucleic acid molecule;
optionally wherein the nucleic acid is selected from the group consisting of a plasmid, a bacterial artificial chromosome, a phagemid, a cosmid, a yeast artificial chromosome, a human artificial chromosome, a viral vector, or any combination thereof,
optionally wherein the nucleic acid is a circular nucleic acid, optionally a plasmid, further comprising an origin of replication and/or further comprising a selectable marker.

26. A cell comprising the optogenetic expression system of any of claims 14-25.

27. The cell of claim 26, wherein the cell:
a) is capable of producing bacterial cellulose;
b) is a bacterial cell, optionally a bacterial cell that expresses all of bcsA, bcsD, bscC and bscD; and/or
c) wherein the cell is a bacterial cell belonging to a genus selected from the group comprising or consisting of: Komagataeibacter, Escherichia, Gluconacetobacter, Acetobacter, Sarcina, Agrobacterium, Azotobacter, Rhizobium, Pseudomonas, Salmonella and Alcaligenes;
optionally
i) wherein the bacterial cell is selected from the group comprising or consisting of: Komagaeibacter rhaeticus Komagaeibacter rhaeticus; Komagaeibacter xylinus, Komagaeibacter hansenii, Komagaeibacter medellinensis, Komagaeibacter europaeus, Komagaelbacter maltaceti, Komagaelbacter pomaceti, Komagaeibacter oboediens, or Komagaelbacter saccharivoans; and/or
ii) the bacterial cell is:
a) a strain of Komagaeibacter rhaeticus selected from the group comprising or consisting of: Komagaeibacter rhaeticus IGEM. Komagaeibacter rhaeticus AF; Komagaeibacter rhaeticus LMG22126, optionally wherein the bacterial cell is a Komagaelbacter rhaeticus iGEM cell; or
b) Gluconacetobacter xylinus CGMCC 2995.

28. The cell of any of either of claim 26 or 27, wherein the first and/or second and/or third and/or fourth nucleic acid of the optogenetic expression system according to any of claims 14-25 is:
a) Integrated into the genome of the cell, optionally wherein:
i) the first and optionally second and optionally fourth nucleic acids of the optogenetic expression system are integrated into the genome of the cell; or
ii) all nucleic acids of the optogenetic expression system are integrated into the genome of the cell;
or
b) maintained episomally within the cell, optionally wherein:
iii) where the first and optionally second and optionally fourth nucleic acids of the optogenetic expression system are integrated into the genome of the cell, the third nucleic acid of the optogenetic system is maintained episomally within the cell; or
iv) all nucleic acids of the optogenetic expression system are maintained episomally within the cell.

29. A method of producing spatially pigmented bacterial cellulose, comprising the steps of:
(a) providing a culture of the cells according to any of claims 26-28 wherein the third nucleotide sequence encodes a polypeptide that:
i) is involved in the biosynthesis of a pigment visible to the naked eye or in the biosynthesis of a molecule that emits light; or
ii) encodes a protein that emits light or is a pigment;
(b) maintaining the culture of (a) under conditions that allow the production of a cellulose pellicle by the microorganism; and
(c) exposing a spatially defined region or regions of the cellulose pellicle to light of the dimerization wavelength so as to allow expression of the third polypeptide.

30. The method according to claim 29 wherein:
a) the cellulose pellicle in (b) is allowed to develop to the final desired area and/or thickness prior to exposing the defined region or regions to light in step (c); and/or
b) once the pellicle in (b) has developed to the final desired area and/or thickness it is harvested prior to exposing the spatially defined region or regions to light in step (c); or
c) the spatially defined regions of the cellulose pellicle are exposed to the light during step (b); and/or
d) the volume of the culture is kept constant during exposure to the light;
e) the region or regions of the cellulose pellicle that are not to be exposed to light are protected using a mask, optionally wherein:
i) the mask is placed as close as possible to the surface of the pellicle, optionally wherein the mask contacts the surface of the pellicle; and/or
ii) the mask is entirely opaque; or
iii) the mask comprises at least some regions that are semi-transparent so as to allow a reduced intensity of light to reach the pellicle in at least some areas.

31. The method according to any of claim 29 or 30 wherein the third polypeptide that:
(a) is involved in the biosynthesis of a pigment visible to the naked eye or in the biosynthesis of a molecule that emits light; or
b) encodes a protein that emits light or is a pigment;
is an enzyme necessary for the formation of melanin, optionally wherein the melanin is selected from the group comprising eumelanin, pheomelanin, neuromelanin and allomelanin; optionally wherein the third polypeptide is tyrosinase, optionally:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.

32. A method for spatially restricted gene expression in bacterial cellulose wherein the method comprises:
(a) providing a culture of the cells according to any of claims 26-28;
(b) maintaining the culture of (a) under conditions that allow the production of a cellulose pellicle by the cells; and
(c) exposing a defined region or regions of the cellulose pellicle to light of the dimerization wavelength so as to allow dimerization of the first and second light-inducible dimerization domain and formation of the functional heterologous polymerase and transcription of the third nucleic acid sequence that encodes a target protein or RNA to be expressed.

33. The method according to claim 32 wherein:
a) the cellulose pellicle in (b) is allowed to develop to the final desired area and/or thickness prior to exposing the defined region or regions to light in step (c); and/or
b) once the pellicle in (b) has developed to the final desired area and/or thickness it is harvested prior to the defined region or regions to light in step (c); or
c) the spatially defined regions of the cellulose pellicle are exposed to the light during step (b); and/or
d) the volume of the culture is kept constant during exposure to the light; and/or
e) the region or regions of the cellulose pellicle that are not to be exposed to light are protected using a mask, optionally wherein:
i) the mask is placed as close as possible to the surface of the pellicle, optionally wherein the mask contacts the surface of the pellicle; and/or
ii) the mask is entirely opaque; or
iii) the mask comprises at least some region or regions that are semi-transparent so as to allow a reduced intensity of light to reach the pellicle in at least some areas.

34. The method according to either of claim 32 or 33 wherein:
a) the strength of expression from the third nucleic acid sequence that encodes a target protein or RNA to be expressed is modulated by varying:
i) the intensity of light that the pellicle or culture is exposed to; and/or
ii) the duration of exposure to light; and/or
b) wherein where:
i) the first nucleic acid comprises a first promoter operably linked so as to drive expression of the first polypeptide; and
ii) the second nucleic acid comprises a second promoter operably linked so as to drive expression of the second polypeptide,
and wherein the first and second promoter are inducible promoters,
then the strength of expression from the third nucleic acid sequence that encodes a target protein or RNA to be expressed is modulated by varying:
a) the intensity of light that the pellicle or culture is exposed to;
b) the duration of exposure to light; and/or
c) the concentration of inducing agent that the pellicle or culture is exposed to;
optionally where the first promoter and second promoter are arabinose inducible promoters, the inducing agent is arabinose.

35. The method according to any of claims 32-34 wherein the third nucleic acid sequence encodes an enzyme necessary for the formation of melanin, optionally wherein the melanin is selected from the group comprising eumelanin, pheomelanin, neuromelanin and allomelanin; optionally wherein the third nucleic acid encodes:
Tyr1 from Bacillus megaterium [SEQ ID NO: 13];
mel from Streptomyces antibiotics [SEQ ID NO: 14]; or
mel from Rhizobium etli [SEQ ID NO: 15];
optionally wherein the tyrosinase comprises or consists of a sequence with at least 80%, 82%, 84%, 86%, 88%, 90%, 92%, 94%, 96%, 98%, 99% or 100% sequence identity to any of the above sequences.

36. A method for producing a bacterial cellulose pellicle that can be spatially pigmented upon exposure to light, wherein the method comprises:
(a) providing a culture of the cells according to any of claims 26-28;
(b) maintaining the culture of (a) under conditions that allow the production of a cellulose pellicle by the microorganism until a pellicle of the appropriate area and/or thickness has been obtained; and
(c) harvesting the pellicle; and
wherein the pellicle has not been exposed to light of the dimerization wavelength.

37. A method for spatially pigmenting bacterial cellulose wherein the method comprises:
a) providing a bacterial cellulose pellicle that has been produced by a culture of cells according to any of claims 26-28, optionally by the method according to claim 36; and
b) exposing spatially restricted areas of the pellicle to light of the dimerization wavelength.

38. The method according to any of claims 29-37, wherein the method further comprises:
exposing the pellicle to a pigment development solution, optionally wherein the pigment development solution:
is at a pH of between 6 and 8.5;
comprises L-tyrosine and/or L-cysteine and/or L-cystine; and
optionally comprises metal ions with an oxidation state of 2+;

39. A spatially pigmented bacterial pellicle as produced according to any method of any of the preceding claims, optionally wherein the pigment is melanin.

40. A pigmented bacterial pellicle as produced according to any method of any of the preceding claims, optionally wherein the pigment is melanin.

41. A bacterial pellicle suitable for light-induced spatially restricted pigmentation wherein the bacterial pellicle has been produced according to the method of claim 36 and wherein the pellicle has not been exposed to light of the dimerization wavelength.

42. The spatially pigmented bacterial pellicle of claim 39, or the bacterial pellicle of claim 41, wherein the bacterial pellicle comprises the cell according to any of claims 26-28.

43. The pigmented bacterial pellicle of claim 40, wherein the bacterial pellicle comprises the cell according to claim 13 or according to any of claims 26-28.

44. A textile comprising a bacterial pellicle according to any of claims 39-43.

45. A pigment development solution, wherein the solution:
a) is at a pH of:
between 6 and 8.5, optionally between 6.25 and 8.25, 6.5 and 8, 6.25 and 7.75, 6.5 and 7.5, 6.75 and 7.25; and/or
at least 6, optionally at least 6.25, 6.5, 6.75, 7, 7.25, 7.4, 7.5, 7.75, 8, 8.25 or at least 8.5; and/or
7.4;
b) comprises L-tyrosine and/or L-cysteine and/or L-cystine; optionally comprises:
i) at least 0.1 g/L tyrosine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L tyrosine; and/or
less than 2 g/L tyrosine, or less than 1.75 g/L, 1.5 g/L, 1 g/L, 0.75 g/L 0.5 g/L, 0.4 g/L, 0.3 g/L, 0.2 g/L, or less than 0.1 g/L tyrosine;
and/or
ii) at least 10 g/L cysteine, optionally at least 20 g/L, 30 g/L, 40 g/L, 50 g/L, 60 g/L, 70 g/L, 80 g/L, 90 g/L, 100 g/L, 120 g/L, 140 g/L, 160 g/L, 180 g/L, 200 g/L, 220 g/L, 240 g/L, 260 g/L, 280 g/L or at least 300 g/L;
between 10 g/L and 300 g/L cysteine; and/or
less than 300 g/L cysteine, or less than 280 g/L, 260 g/L, 240 g/L, 220 g/L 200 g/L, 180 g/L, 160 g/L, 140 g/L, 120 g/L, 100 g/L, 90 g/L, 80 g/L 70 g/L, 60 g/L, 50 g/L, 40 g/L, 30 g/L, 20 g/L or less than 10 g/L cysteine;
and/or
iii) at least 0.1 g/L L-cystine, optionally at least 0.2 g/L, 0.3 g/L, 0.4 g/L, 0.5 g/L, 0.75 g/L, 1 g/L, 1.5 g/L, 1.75 g/L or at least 2 g/L;
between 0.1 g/L and 2 g/L L-cystine; and/or
less than 2 g/L L-cystine, or less than 1.75 g/L, 1.5 g/L, 1 g/L, 0.75 g/L 0.5 g/L, 0.4 g/L, 0.3 g/L, 0.2 g/L, or less than 0.1 g/L L-cystine; and optionally
c) comprises metal ions with an oxidation state of 2+; optionally comprises:
i) a) Cu²⁺, Zn²⁺, Be²⁺, Mg²⁺, Ca²⁺, Cr²⁺, Mn²⁺, Co²⁺ or Ni²⁺;
b) Cu²⁺, Zn²⁺; and/or
c) Cu²⁺; and/or
ii) CUSO₄, optionally comprises:
at least 2 μM CuSO₄, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuSO₄; and/or
between 2 μM CUSO₄ and 20 μM CUSO₄; and/or
less than 20 μM, optionally less than 17.5 μM, 15 μM, 12.5 μM, 10 μM, 7.5 μM, 5 μM, 4 μM, 3 μM, or less than 2 μM; and/or
between 20 μM and 160 μM CuSO₄; and/or
less than 160 μM, optionally less than 140 μM, 120 μM, 100 μM, 80 μM, 60 μM, 40 μM, or less than 20 μM CuSO₄; and/or
iii) CuCl₂, optionally comprises:
at least 2 μM CUCl₂, optionally at least 3 μM, 4 μM, 5 μM, 7.5 μM, 10 μM, 12.5 μM, 15 μM, 17.5 μM or at least 20 μM, 40 μM, 60 μM, 80 μM, 100 μM, 120 μM, 140 μM, or at least 160 μM CuCl₂; and/or
between 2 μM CuCl₂ and 20 μM CUCl₂; and/or
less than 20 μM, optionally less than 17.5 μM, 15 μM, 12.5 μM, 10 μM, 7.5 μM, 5 μM, 4 μM, 3 μM, or less than 2 μM;
between 20 μM and 160 μM CuCl₂; and/or
less than 160 μM, optionally less than 140 μM, 120 μM, 100 μM, 80 μM, 60 μM, 40 μM, or less than 20 μM CuCl₂;
optionally wherein the development solution comprises:
a) PBS at pH 7.4;
b) 10 μM CuSO₄ or 20 μM CuSO₄; and
c) 0.5 g/L L-tyrosine, 1 g/L L-tyrosine, and/or 1 g/L cysteine and/or 0.4 g/L cystine.

46. A kit comprising:
a) a bacterial pellicle as described in any of the above claims; and
b) a development solution according to any of the preceding claims;
and optionally comprises
c) a light source capable of emitting light of the wavelength of the light results in dimerization of the first and second dimerization domains,
optionally wherein the bacterial pellicle comprises an optogenetic expression system according to any of the preceding claims.
